(19) United States
(12) Patent Application Publication
(10) Pub. No.: US 2024/0262804 A1
(43) Pub. Date: Aug. 8, 2024

(54) NITROGEN-CONTAINING HETEROCYCLIC KETONES, PREPARATION METHODS AND MEDICINAL USES THEREOF

(71) Applicants: Hansoh Bio LLC, Rockville, MD (US); Shanghai Hansoh Biomedical Co., Ltd., Shanghai (CN); Jiangsu Hansoh Pharmaceutical Group Co., Ltd., Lianyungang, Jiangsu (CN)

(72) Inventors: Hugh Y. Zhu, Rockville, MD (US); Gang Liu, Rockville, MD (US); Che Liu, Rockville, MD (US)

(21) Appl. No.: 18/550,666

(22) PCT Filed: Mar. 17, 2022
(86) PCT No.: PCT/CN2022/081361

§ 371 Date: Sep. 14, 2023

Related U.S. Application Data
(60) Provisional application No. 63/162,125, filed on Mar. 17, 2021.
(60) Provisional application No. 63/265,004, filed on Dec. 6, 2021.

(51) Int. Cl. C07D 401/04; A61K 31/53; A61P 9/04; C07D 251/08; C07D 403/04; C07D 405/04; C07D 405/14; C07D 409/12; C07D 487/04; C07F 7/08

(52) U.S. Cl. CPC C07D 401/04; A61K 31/53; A61P 9/04; C07D 251/08; C07D 403/04; C07D 405/04; C07D 405/14; C07D 409/12; C07D 487/04; C07F 7/0834; C07B 2200/09

(57) ABSTRACT

Provided herein is novel N-heterocyclic ketones that are useful for treatment of hypertrophic cardiomyopathy (HCM) and other heart diseases. The preparation method thereof, pharmaceutical compositions comprising the compound.

### FIELD OF THE INVENTION

The present invention belongs to the field of medicine, and relates to nitrogen-containing heterocyclic ketones, preparation methods thereof, pharmaceutical compositions comprising the compounds, and medical uses thereof.

### BACKGROUND OF THE INVENTION

Hypertrophic cardiomyopathy (HCM) is a genetic disease with an incidence of 1 in around 500 individuals in the general population. HCM patients are often diagnosed with clinical observation of left ventricle hypertrophy that cannot be explained by other known causes. Other notable histopathologic findings of HCM include enlarged, disorganized cardiomyocytes and increased amounts of myocardial fibrosis. The heart function of HCM patient is also perturbed with characteristically hyperdynamic contraction and impaired relaxation.

HCM patient with underlying familial or somatic mutations may show symptoms including chest pain, shortness of breath, fatigue, palpitations, and even sudden death.

Albeit its prevalence and serious symptoms, available targeted therapies to ameliorate HCM at its source and to alter the progression of the disease are rare. Current off label use of medications, such as beta-adrenergic receptor blockers or calcium channel blockers, could non-specifically reduce the contractility of the heart muscles and thus provide some symptom relief, but the progression of disease could not be altered by these treatments. There is a great need for pharmaceutical agents that could suppress the development of ventricular hypertrophy, cardiomyocyte disarray, and myocardial fibrosis.

Selective inhibition of the hypercontractility of cardiac sarcomere is a promising targeted approach for HCM. The new mechanisms of action may offer therapeutical advantages in terms of relief of symptoms, improved therapeutical window, and reduction of patient mortality. Accordingly, there is a need in the art for novel selective cardiac sarcomere modulators.

### SUMMARY OF THE INVENTION

Selective cardiac sarcomere modulators, such as cardiac myosin inhibitors, have been identified as effective agents to treat HCM in both preclinical and clinical settings. The present disclosure provides such agents and methods for their use.

In one general aspect, the present invention, in one aspect, provides a compound of formula (I), or a pharmaceutically acceptable salt, solvate, or prodrug thereof, including tautomers, cis- or trans-isomers, mesomers, racemates, enantiomers, diastereomers, and mixtures thereof:

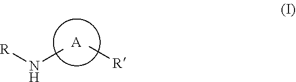

wherein:

A is selected from the group consisting of:

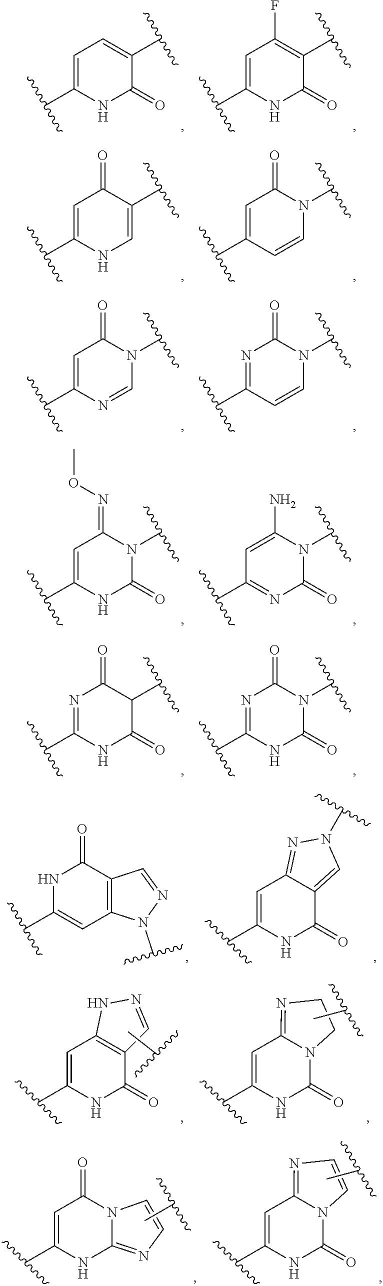

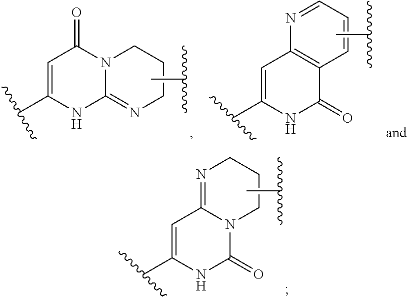

R is —(CR₁R₂)ₙR₃;
R₁ and R₂ are independently selected from the group consisting of hydrogen, deuterium, halogen, amino, cyano, hydroxyl, alkyl, alkoxy, haloalkyl, hydroxyalkyl, cycloalkyl, heterocyclyl, aryl and heteroaryl;
n is 0, 1, 2, 3 or 4;
R₃ is selected from the group consisting of hydrogen, deuterium, halogen, amino, cyano, hydroxyl, alkyl, alkoxy, haloalkyl, hydroxyalkyl, cycloalkyl, heterocyclyl, aryl and heteroaryl, wherein each of the alkyl, alkoxy, haloalkyl, hydroxyalkyl, cycloalkyl, heterocyclyl, aryl and heteroaryl at each occurrence is independently unsubstituted or substituted with one or more substituents selected from the R³ group consisting of deuterium, halogen, amino, nitro, oxo, cyano, hydroxy, alkyl, alkoxy, haloalkyl, hydroxyalkyl, —NRₐR_{b}, —C(O)Rₐ, —C(O)NRₐR_{b}, —C(O)ORₐ, —OC(O)Rₐ, —S(O)ₘRₐ, —S(O)ₘNRₐR_{b}, cycloalkyl, heterocyclyl, aryl and heteroaryl, wherein the alkyl, hydroxyalkyl, cycloalkyl, heterocyclyl, aryl, and heteroaryl in said R³ group of substituents is independently unsubstituted or substituted with one or more substituents selected from alkyl, haloalkyl, cyano, —C(O)Rₐ, halogen, and cycloalkyl;
m is 0, 1 or 2;
   R′ is selected from the group consisting of alkyl, alkoxy, haloalkyl, hydroxyalkyl, cycloalkyl, heterocyclyl, aryl and heteroaryl, wherein each of the alkyl, alkoxy, haloalkyl, hydroxyalkyl, cycloalkyl, heterocyclyl, aryl and heteroaryl at each occurrence is independently unsubstituted or substituted with one or more substituents selected from the group consisting of deuterium, halogen, amino, cyano, hydroxy, alkyl, alkoxy, haloalkyl, hydroxyalkyl, —NR_{c}R_{d}, —C(O)R_{c}, —C(O)NR_{c}R_{d}, —C(O)OR_{c}, —OC(O)R_{c},
—S(O)ₘR_{c} and —S(O)ₘNR_{c}R_{d};
Rₐ, R_{b}, R_{c}, and R_{d} are independently selected from the group consisting of hydrogen, deuterium, halogen, amino, cyano, hydroxy, alkyl, alkoxy, haloalkyl and hydroxyalkyl.

In an embodiment, the compound of formula (I) is a compound of formula (II), or a tautomer, cis- or trans-isomer, mesomer, racemate, enantiomer, diastereomer, or mixture thereof, or a pharmaceutically acceptable salt, solvate, or prodrug thereof,

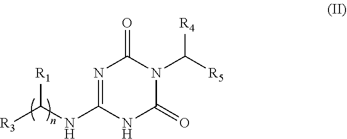

wherein,
R₁ is selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl; R₃ is selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl comprising one or more of the members of N, O, S and S(O)₂, C₆-C₁₂ aryl, and 4-8 membered heteroaryl comprising one or more of the members of N, O, S and S(O)₂ wherein each of the C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl at each occurrence is independently unsubstituted or substituted with one or more substituents selected from the R³ group consisting of deuterium, halogen, amino, nitro, oxo, cyano, hydroxyl, C₁-C₆ alkyl, C₁-C₆ alkoxy, C₁-C₆ haloalkyl, C₁-C₆ hydroxyalkyl, —NRₐR_{b}, —C(O)Rₐ, —C(O)NRₐR_{b}, —C(O)ORₐ, —OC(O)Rₐ, —S(O)ₘRₐ, and —S(O)ₘNRₐR_{b}, wherein the C₃-C₆ cycloalkyl, 4-6 membered heterocyclyl comprising one or more of the members of N, O, S and S(O)₂, phenyl, 4-6 membered heteroaryl comprising one or more of the members of N, O, S and S(O)₂, C₁-C₆ alkyl, and C₁-C₆ hydroxyalkyl in said R³ group of substituents is independently unsubstituted or substituted with one or more substituents selected from C₁-C₆ alkyl, C₁-C₆ haloalkyl, cyano, —C(O)Rₐ, halogen, and C₃-C₆ cycloalkyl;
R₄ and R₅ are independently selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl comprising one or more of the members of N, O, S and S(O)₂, C₆-C₁₂ aryl, and 4-8 membered heteroaryl comprising one or more of the members of N, O, S or S(O)₂, wherein each of the C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl at each occurrence is independently unsubstituted or substituted with one or more substituents selected from the group consisting of deuterium, halogen, amino, cyano, hydroxyl, C₁-C₆ alkyl, C₁-C₆ alkoxy, C₁-C₆ haloalkyl, C₁-C₆ hydroxyalkyl, —NR_{c}R_{d}, —C(O)R_{c}, —C(O)NR_{c}R_{d}, —C(O)OR_{c} and —OC(O)R_{c};
or, R₄ and R₅ together with the C atom to which they are bound form a cyclic structure selected from the R⁴⁵Cycle group consisting of C₃-C₈ cycloalkyl, 4-8 membered heterocyclyl comprising one or more of the members of N and O, C₆-C₁₂ aryl, and 4-8 membered heteroaryl comprising one or more of the members of N and O, wherein each of the cyclic structures in said R⁴⁵Cycle group is optionally substituted with one to four substituents selected from the group consisting of deuterium, halogen, amino, cyano, hydroxyl, C₁-C₆ alkyl, C₁-C₆ haloalkyl, C₁-C₆ alkoxy, C₁-C₆ hydroxyalkyl, —N_{c}R_{d}, —C(O)R_{c}, —C(O)N_{c}R_{d}, —C(O)OR_{c}, and —OC(O)R_{c};
Rₐ, R_{b}, R_{c}, and R_{d} are independently selected from the group consisting of hydrogen, deuterium, halogen, amino, cyano, hydroxyl, C₁-C₆ alkyl, C₁-C₆ alkoxy, C₁-C₆ haloalkyl, and C₁-C₆ hydroxyalkyl.

In some embodiments, R₄ and R₅ together with the C atom to which they are bound form a 4-8 membered heterocyclyl comprising an N atom.

In some embodiments, R₁ is selected from the group consisting of hydrogen, hydroxyl, C₁-C₃ alkyl, C₁-C₃ haloalkyl, C₁-C₃ alkoxy, and C₁-C₃ hydroxyalkyl.

In some embodiments, R₁ is H, —OH, —CH₃, —CH₂CH₃, —CH(CH₃)₂, —CH₂OH, —CF₃, or

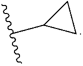 [SMILES: *C1CC1]

In some embodiments, R₃ is selected from the group consisting of C₁-C₃ alkyl, C₁-C₃ haloalkyl, C₁-C₃ alkoxy, C₁-C₃ hydroxyalkyl, C₃-C₆ cycloalkyl, phenyl, 5-6 membered heterocyclyl comprising 1-2 of the members of N, O, S and S(O)₂, and 5-6 membered heteroaryl comprising 1-2 of the members of N, O, S and S(O)₂, wherein each of the substituents in said R₃ is optionally substituted with one to two substituents selected from the R³ group consisting of deuterium, halogen, amino, nitro, oxo, cyano, hydroxyl, C₁-C₃ alkyl, C₁-C₃ alkoxy, C₁-C₃ haloalkyl, C₁-C₃ hydroxyalkyl, 4-6 membered heterocyclyl comprising one or more of the members of N, O, S and S(O)₂, —C(O)Rₐ, —C(O)NRₐR_{b}, —S(O)₂Rₐ, and —S(O)₂NRₐR_{b}, wherein the C₁-C₃ alkyl, C₁-C₃ hydroxyalkyl and 4-6 membered heterocyclyl comprising one or more of the members of N, O, S and S(O)₂, in said R³ group of substituents is independently unsubstituted or substituted with one or more substituents selected from C₁-C₃ alkyl, C₁-C₃ haloalkyl, cyano, —C(O)Rₐ, halogen, and C₃-C₆ cycloalkyl;

In some embodiments, Rₐ and R_{b} are independently selected from the group consisting of hydrogen, deuterium, halogen, amino, cyano, hydroxyl, C₁-C₃ alkyl, C₁-C₃ alkoxy, C₁-C₃ haloalkyl, and C₁-C₃ hydroxyalkyl.

In some embodiments, R₄ and R₅ are independently selected from the group consisting of hydrogen, deuterium, halogen, amino, cyano, hydroxyl, C₁-C₃ alkyl, C₁-C₃ alkoxy, C₁-C₃ haloalkyl, C₁-C₃ hydroxyalkyl, C₃-C₆ cycloalkyl, 5-6 membered heterocyclyl comprising 1-2 of the members of N, O, S and S(O)₂, C₆-C₁₂ aryl, and 5-6 membered heteroaryl comprising 1-2 of the members of N, O, S and S(O)₂, wherein each of the C₁-C₃ alkyl, C₁-C₃ alkoxy, C₁-C₃ haloalkyl, C₁-C₃ hydroxyalkyl, C₃-C₆ cycloalkyl, 5-6 membered heterocyclyl, C₆-C₁₂ aryl and 5-6 membered heteroaryl at each occurrence is independently unsubstituted or substituted with one or more substituents selected from the group consisting of deuterium, halogen, amino, cyano, hydroxyl, C₁-C₃ alkyl, C₁-C₃ alkoxy, C₁-C₃ haloalkyl, C₁-C₃ hydroxyalkyl, —NR_{c}R_{d}, —C(O)R_{c}, —C(O)NR_{c}R_{d}, —C(O)OR_{c}, and —OC(O)R_{c}.

In some embodiments, R₄ and R₅ together with the C atom to which they are bound form a cyclic structure selected from the C⁴⁵Cycle (II) group consisting of a C₃-C₆ cycloalkyl, 5-6 membered heterocyclyl comprising 1-2 of the members of N and O atom, phenyl, and 5-6 membered heteroaryl comprising 1-2 of the members of N and O atom, wherein each of the cyclic structures in said C⁴⁵Cycle (II) group is optionally substituted with one or two substituents selected from the group consisting of deuterium, halogen, amino, cyano, hydroxyl, C₁-C₃ alkyl, C₁-C₃ haloalkyl, C₁-C₃ alkoxy and C₁-C₃ hydroxyalkyl, —NR_{c}R_{d}, —C(O)R_{c}, —C(O)NR_{c}R_{d}, —C(O)OR_{c}, and —OC(O)R_{c}.

In some embodiments, R_{c} and R_{d} are independently selected from the group consisting of hydrogen, deuterium, halogen, amino, cyano, hydroxyl, C₁-C₃ alkyl, C₁-C₃ alkoxy, C₁-C₃ haloalkyl, and C₁-C₃ hydroxyalkyl.

In some embodiments, R₄ and R₅ are independently selected from —CH₃ and —CF₃.

In some embodiments, R₄ and R₅ are —CH₃.

In some embodiments, R₄ and R₅ together with the C atom to which they are bound form a cyclic structure selected from the RCycle group consisting of:

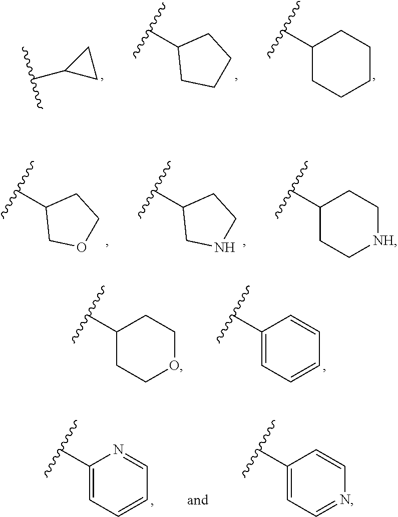

wherein each of the cyclic structures in said RCycle group is optionally substituted with one or two substituents selected from the group consisting of —F, —Cl, —Br, —OH, —CH₃, —CH₂CH₃, —CF₃, and —C(O)CH₃.

In some embodiments, n is 0, 1 or 2.

In some embodiments, n is 1.

In some embodiments, the compound of formula (II) is a compound of formula (III), or a tautomer, cis- or trans-isomer, mesomer, racemate, enantiomer, diastereomer, or mixture thereof, or a pharmaceutically acceptable salt, solvate, or prodrug thereof,

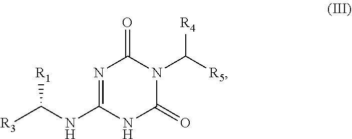

wherein R₁, R₃, R₄ and R₅ are defined as in formula (II).

In some embodiments, when each of R₄ and R₅ is methyl, then n is 0, and R₃ is neither

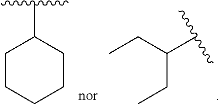

In some embodiments, when each of R₁, R₄ and R₅ is methyl, then n is 1, and R₃ is not

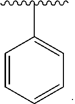 [SMILES: *c1ccccc1]

In some embodiments, when R₄ and R₅ with the C atom to which they are bound form

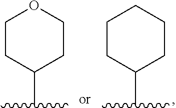

R₁ is methyl, then n is 1, and R₃ is not

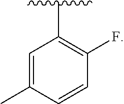 [SMILES: *c1cc(C)ccc1F]

In some embodiments, the compound of formula (III) is a compound of formula (IV), or a tautomer, cis- or trans-isomer, mesomer, racemate, enantiomer, diastereomer, or mixture thereof, or a pharmaceutically acceptable salt, solvate, or prodrug thereof,

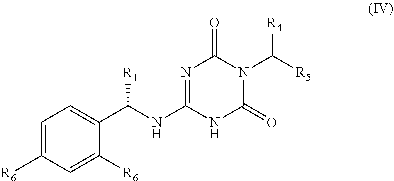

wherein,
R₁ is C₁-C₃ alkyl, C₁-C₃ haloalkyl or C₁-C₃ alkoxy;
R₄ and R₅ together with the C atom to which they are bound form a 5-6 membered heterocyclyl comprising 1-2 of the members of N and O; and
R₆ is independently selected from the group consisting of halogen, C₁-C₃ alkyl, C₁-C₃ alkoxy and C₁-C₃ haloalkyl.

In some embodiments of the compound of formula (IV), or a tautomer, cis- or trans-isomer, mesomer, racemate, enantiomer, diastereomer, or mixture thereof, or a pharmaceutically acceptable salt, solvate, or prodrug thereof:
R₁ is C₁-C₃ alkyl;
R₄ and R₅ together with the C atom to which they are bound form

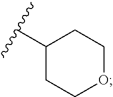 [SMILES: *C1CCOCC1]

and
R₆ is independently selected from the group consisting of F, Cl and Br.

The present invention also provides a pharmaceutical composition, comprising a therapeutically effective amount of a compound of any formula described herein, or a tautomer, cis- or trans isomer, mesomer, racemate, enantiomer, diastereomer, or mixture thereof, or a pharmaceutically acceptable salt, solvate, or prodrug thereof, together with one or more pharmaceutically acceptable carriers, diluents or excipients. In another aspect, the present invention relates to a method of treating hypertrophic cardiomyopathy (HCM) or a cardiac disorder having a pathophysiological feature of HCM in a subject in need thereof, comprising administering to the subject an effective amount of a compound of any formula described herein or a pharmaceutical composition comprising the same.

In a preferred embodiment, the HCM is obstructive or nonobstructive or is caused by sarcomeric and/or non-sarcomeric mutations.

In another aspect, the present invention relates to a method of treating a disease or disorder selected from the group consisting of heart failure with preserved ejection fraction, ischemic heart disease, angina pectoris, and restrictive cardiomyopathy, comprising administering to a subject in need thereof an effective amount of a compound any formula described herein or a pharmaceutical composition comprising the same.

### BRIEF DESCRIPTION OF THE DRAWINGS

FIG. 1: The effect of compound of Example 4 on heart function was measured in Spraw-Dawley rats at different doses.

FIG. 2: The effect of compound of Example 10 on heart function was measured in Spraw-Dawley rats at different doses.

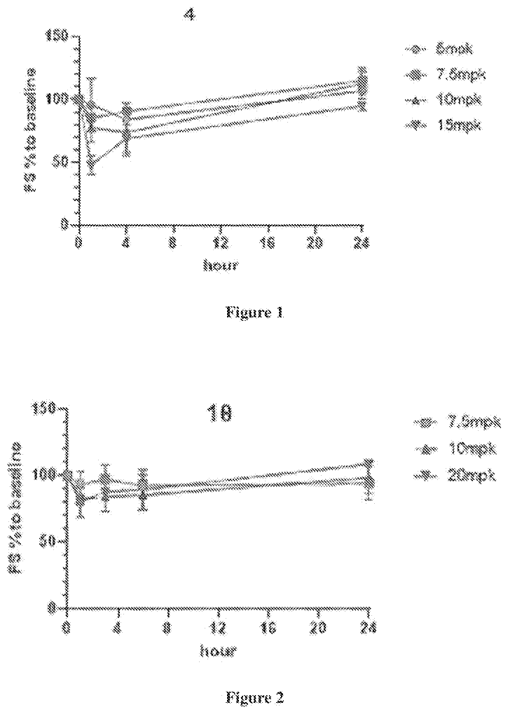

### DETAILED DESCRIPTION OF THE INVENTION

Various publications, articles and patents are cited or described through the specification; each of these references is herein incorporated by references in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Given below are definitions of terms used in this invention. Any term not defined herein takes the normal meaning as the skilled person would understand the term.

Where it is stated that groups or substituents are “independently selected from” (and variants thereof) a list of choices, it is meant that the choice for any one of such groups or substituents does not determine the choice for any other one of such groups or substituents. By way of an illustration, but not as a limitation, the term “A and B are independently selected from a and b” or “each of A and B is independently selected from a and b” is meant to encompass selections where A is a and B is a, A is b and B is b, A is a and B is b, and A is b and B is a.

It must be noted that as used herein and in the appended claims, the singular forms “a,” “an,” and “the” include plural reference unless the context clearly dictates otherwise.

Unless otherwise indicated, the term “at least” preceding a series of elements is to be understood to refer to every element in the series. For example, the phrase “at least A, B, and C” means that each of A, B, and C is present. The term “at least one of” preceding a series of elements is to be understood to refer to a single element in the series or any combination of two or more elements in the series. For example, the phrase “at least one of A, B, and C” means that only A is present, only B is present, only C is present, both A and B are present, both A and C are present, both B and C are present, or each of A, B, and C is present. Depending on the context, “at least one of” preceding a series of elements can also encompass situations in which any one or more of 3 the elements is present in greater than one instance, e.g., “at least one of A, B, and C” can also encompass situations in which A is present in duplicate alone or further in combination with any one or more of elements B and C.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/of” as used herein. Concurrent applicability of more than one of the options is also understood to fall within the meaning, and therefore satisfy the requirement of the term “and/or.” “Alkyl” refers to a saturated aliphatic hydrocarbon group including C₁-C₂₀ straight chain and branched chain groups. Preferably an alkyl group is an alkyl having 1 to 12, sometimes preferably 1 to 6, sometimes more preferably 1 to 4, carbon atoms.

Representative examples include, but are not limited to methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, n-heptyl, 2-methylhexyl, 3-methylhexyl, 4-methylhexyl, 5-methylhexyl, 2,3-dimethylpentyl, 2,4-dimethylpentyl, 2,2-dimethylpentyl, 3,3-dimethylpentyl, 2-ethylpentyl, 3-ethylpentyl, n-octyl, 2,3-dimethylhexyl, 2,4-dimethylhexyl, 2,5-dimethylhexyl, 2,2-dimethylhexyl, 3,3-dimethylhexyl, 4,4-dimethylhexyl, 2-ethylhexyl, 3-ethylhexyl, 4-ethylhexyl, 2-methyl-2-ethylpentyl, 2-methyl-3-ethylpentyl, n-nonyl, 2-methyl-2-ethylhexyl, 2-methyl-3-ethylhexyl, 2,2-diethylpentyl, n-decyl, 3,3-diethylhexyl, 2,2-diethylhexyl, and the isomers of branched chain thereof. More preferably an alkyl group is a lower alkyl having 1 to 6 carbon atoms. Representative examples include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc.

The alkyl group can be substituted or unsubstituted. When substituted, the substituent group(s) can be substituted at any available connection point, preferably the substituent group(s) is one or more substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkenyl” refers to an alkyl defined as above that has at least two carbon atoms and at least one carbon-carbon double bond, for example, vinyl, 1-propenyl, 2-propenyl, 1-, 2-, or 3-butenyl, etc., preferably C₂₋₂₀ alkenyl, more preferably C₂₋₁₂ alkenyl, and most preferably C₂₋₆ alkenyl. The alkenyl group can be substituted or unsubstituted. When substituted, the substituent group(s) is preferably one or more, sometimes preferably one to five, sometimes more preferably one to three, group(s) independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Alkynyl” refers to an alkyl defined as above that has at least two carbon atoms and at least one carbon-carbon triple bond, for example, ethynyl, 1-propynyl, 2-propynyl, 1-, 2-, or 3-butynyl etc., preferably C₂₋₂₀ alkynyl, more preferably C₂₋₁₂ alkynyl, and most preferably C₂₋₆ alkynyl. The alkynyl group can be substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkylene” refers to a saturated linear or branched aliphatic hydrocarbon group, wherein having 2 residues derived by removing two hydrogen atoms from the same carbon atom of the parent alkane or two different carbon atoms. The straight or branched chain group containing 1 to 20 carbon atoms, preferably has 1 to 12 carbon atoms, more preferably 1 to 6 carbon atoms. Non-limiting examples of alkylene groups include, but are not limited to, methylene (—CH₂—), 1,1-ethylene (—CH(CH₃)—), 1,2-ethylene (—CH₂CH₂)—, 1,1-propylene (—CH(CH₂CH₃)—), 1,2-propylene (—CH₂CH(CH₃)—), 1,3-propylene (—CH₂CH₂CH₂—), 1,4-butylidene (—CH₂CH₂CH₂CH₂—) etc. The alkylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio. “Alkenylene” refers to an alkylene defined as above that has at least two carbon atoms and at least one carbon-carbon double bond, preferably C₂₋₂₀ alkenylene, more preferably C₂₋₁₂ alkenylene, and most preferably C₂₋₆ alkenylene. Non-limiting examples of alkenylene groups include, but are not limited to, —CH═CH—, —CH═CHCH₂—, —CH═CHCH₂CH₂—, —CH₂CH═CHCH₂— etc. The alkenylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Alkynylene” refers to an alkynyl defined as above that has at least two carbon atoms and at least one carbon-carbon triple bond, preferably C₂₋₂₀ alkynylene, more preferably C₂₋₁₂ alkynylene, and most preferably C₂₋₆ alkynylene. Non-limiting examples of alkenylene groups include, but are not limited to, —CH═CH—, —CH═CHCH₂—, —CH═CHCH₂CH₂—, —CH₂CH═CHCH₂— etc. The alkynylene group can be substituted or unsubstituted. When substituted, the substituent group(s) is preferably one or more, sometimes preferably one to five, sometimes more preferably one to three, group(s) independently selected from the group consisting of selected from alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.

“Cycloalkyl” refers to a saturated and/or partially unsaturated monocyclic or polycyclic hydrocarbon group having 3 to 20 carbon atoms, preferably 3 to 12 carbon atoms, more preferably 3 to 10 carbon atoms, and most preferably 3 to 8 carbon atoms or 3 to 6 carbon atoms. Representative examples of monocyclic cycloalkyls include, but are not limited to, cyclopropyl, cyclobutyl, cyclopentyl, cyclopentenyl, cyclohexyl, cyclohexenyl, cyclohexadienyl, cycloheptyl, cycloheptatrienyl, cyclooctyl, etc. Polycyclic cycloalkyl includes a cycloalkyl having a spiro ring, fused ring or bridged ring.

“Spiro Cycloalkyl” refers to a 5 to 20 membered polycyclic group with rings connected through one common carbon atom (called a spiro atom), wherein one or more rings can contain one or more double bonds, but none of the rings has a completely conjugated pi-electron system. Preferably a spiro cycloalkyl is 6 to 14 membered, and more preferably 7 to 10 membered. According to the number of common spiro atoms, a spiro cycloalkyl is divided into mono-spiro cycloalkyl, di-spiro cycloalkyl, or poly-spiro cycloalkyl, and preferably refers to a mono-spiro cycloalkyl or di-spiro cycloalkyl, more preferably 4-membered/4-membered, 4-membered/5-membered, 4-membered/6-membered, 5-membered/5-membered, or 5-membered/6-membered mono-spiro cycloalkyl. Representative examples of spiro cycloalkyl include, but are not limited to the following substituents:

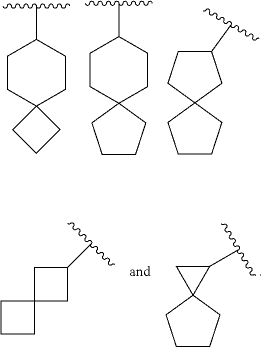

“Fused Cycloalkyl” refers to a 5 to 20 membered polycyclic hydrocarbon group, wherein each ring in the system shares an adjacent pair of carbon atoms with another ring, wherein one or more rings can contain one or more double bonds, but none of the rings has a completely conjugated pi-electron system. Preferably, a fused cycloalkyl group is 6 to 14 membered, more preferably 7 to 10 membered. According to the number of membered rings, fused cycloalkyl is divided into bicyclic, tricyclic, tetracyclic or polycyclic fused cycloalkyl, and preferably refers to a bicyclic or tricyclic fused cycloalkyl, more preferably 5-membered/5-membered, or 5-membered/6-membered bicyclic fused cycloalkyl. Representative examples of fused cycloalkyls include, but are not limited to, the following substituents:

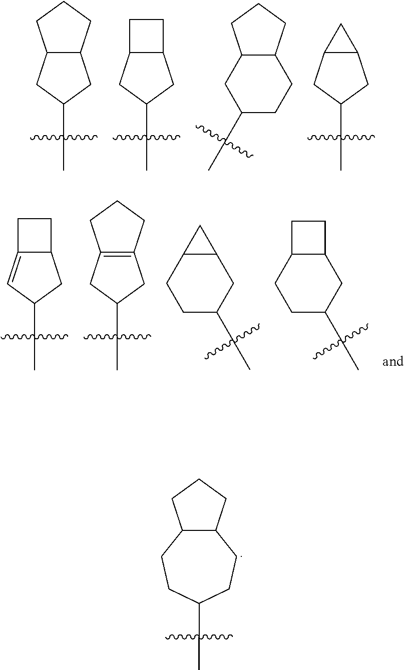

“Bridged Cycloalkyl” refers to a 5 to 20 membered polycyclic hydrocarbon group, wherein every two rings in the system share two disconnected carbon atoms. The rings can have one or more double bonds, but have no completely conjugated pi-electron system. Preferably, a bridged cycloalkyl is 6 to 14 membered, and more preferably 7 to 10 membered. According to the number of membered rings, bridged cycloalkyl is divided into bicyclic, tricyclic, tetracyclic or polycyclic bridged cycloalkyl, and preferably refers to a bicyclic, tricyclic or tetracyclic bridged cycloalkyl, more preferably a bicyclic or tricyclic bridged cycloalkyl. Representative examples of bridged cycloalkyls include, but are not limited to, the following substituents:

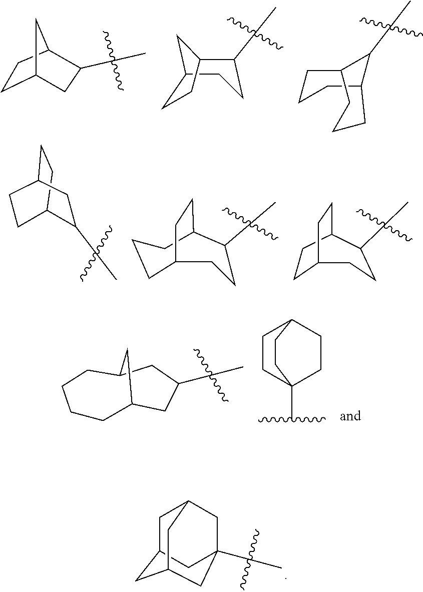

The cycloalkyl can be fused to the ring of an aryl, heteroaryl or heterocyclic alkyl, wherein the ring bound to the parent structure is cycloalkyl. Representative examples include, but are not limited to indanylacetic, tetrahydronaphthalene, benzocycloheptyl and so on:

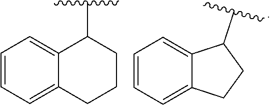

The cycloalkyl is optionally substituted or unsubstituted. When substituted, the substituent group(s) is preferably one or more, sometimes preferably one to five, sometimes more preferably one to three, substituents independently selected from the group consisting of alkyl, halogen, alkoxy, alkenyl, alkynyl, alkylsulfo, alkylamino, thiol, hydroxy, nitro, cyano, amino, cycloalkyl, heterocyclic alkyl, aryl, heteroaryl, cycloalkoxyl, heterocylic, cycloalkylthio, heterocylic alkylthio and oxo group.

“Heterocyclyl” refers to a 3 to 20 membered saturated and/or partially unsaturated monocyclic or polycyclic hydrocarbon group having one or more, sometimes preferably one to five, sometimes more preferably one to three, heteroatoms selected from the group consisting of N, O, and S(O)ₘ (wherein m is 0, 1, or 2) as ring atoms, but excluding —O—O—, —O—S— or —S—S— in the ring, the remaining ring atoms being C. Preferably, heterocyclyl is a 3 to 12 membered having 1 to 4 heteroatoms; more preferably a 3 to 10 membered having 1 to 3 heteroatoms; most preferably a 5 to 6 membered having 1 to 2 heteroatoms. Representative examples of monocyclic heterocyclyls include, but are not limited to, pyrrolidyl, piperidyl, piperazinyl, morpholinyl, sulfo-morpholinyl, homopiperazinyl, and so on. Polycyclic heterocyclyl includes the heterocyclyl having a spiro ring, fused ring or bridged ring.

“Spiro heterocyclyl” refers to a 5 to 20 membered polycyclic heterocyclyl with rings connected through one common carbon atom (called a spiro atom), wherein said rings have one or more, sometimes preferably one to five, sometimes more preferably one to three, heteroatoms selected from the group consisting of N, O, and S(O)ₘ (wherein m is 0, 1 or 2) as ring atoms, the remaining ring atoms being C, wherein one or more rings can contain one or more double bonds, but none of the rings has a completely conjugated pi-electron system. Preferably a spiro heterocyclyl is 6 to 14 membered, and more preferably 7 to 10 membered. According to the number of common spiro atoms, spiro heterocyclyl is divided into mono-spiro heterocyclyl, di-spiro heterocyclyl, or poly-spiro heterocyclyl, and preferably refers to mono-spiro heterocyclyl or di-spiro heterocyclyl, more preferably 4-membered/4-membered, 4-membered/5-membered, 4-membered/6-membered, 5-membered/5-membered, or 5-membered/6-membered mono-spiro heterocyclyl. Representative examples of spiro heterocyclyl include, but are not limited to the following substituents:

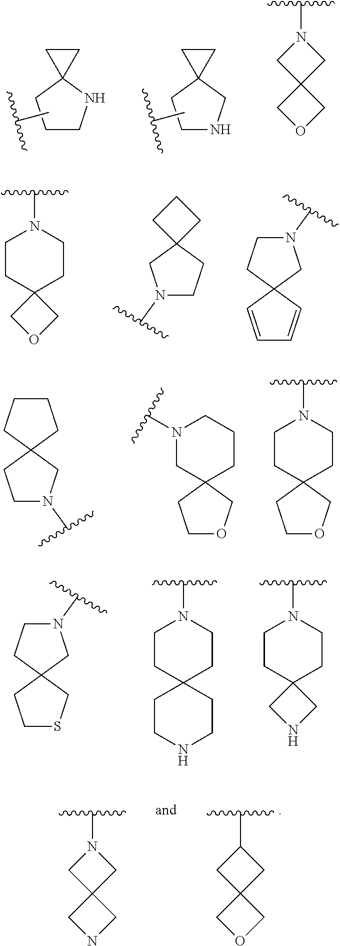

“Fused Heterocyclyl” refers to a 5 to 20 membered polycyclic heterocyclyl group, wherein each ring in the system shares an adjacent pair of carbon atoms with the other ring, wherein one or more rings can contain one or more double bonds, but none of the rings has a completely conjugated pi-electron system, and wherein said rings have one or more, sometimes preferably one to five, sometimes more preferably one to three, heteroatoms selected from the group consisting of N, O, and S(O)ₚ (wherein p is 0, 1, or 2) as ring atoms, the remaining ring atoms being C. Preferably a fused heterocyclyl is 6 to 14 membered, and more preferably 7 to 10 membered. According to the number of membered rings, fused heterocyclyl is divided into bicyclic, tricyclic, tetracyclic or polycyclic fused heterocyclyl, preferably refers to bicyclic or tricyclic fused heterocyclyl, more preferably 5-membered/5-membered, or 5-membered/6-membered bicyclic fused heterocyclyl. Representative examples of fused heterocyclyl include, but are not limited to, the following substituents:

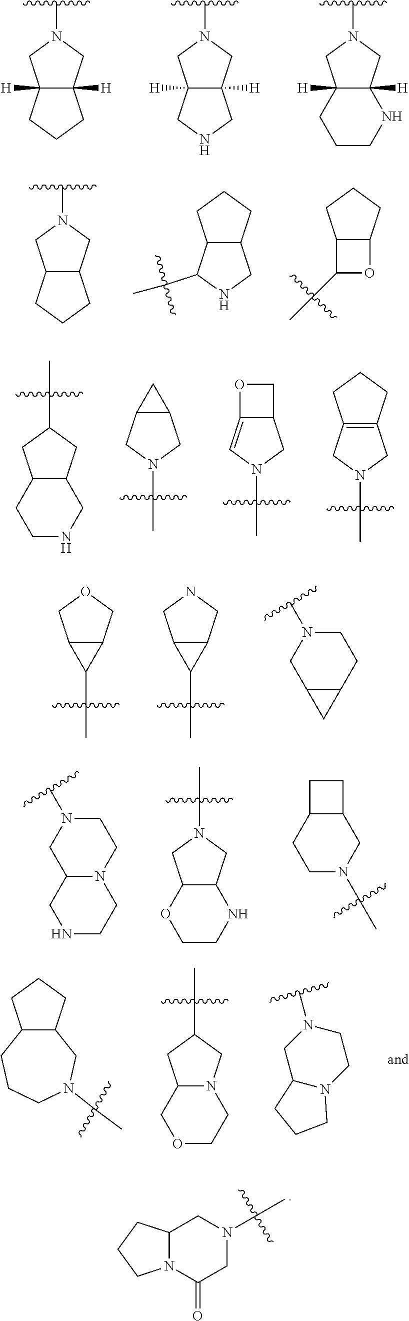

“Bridged Heterocyclyl” refers to a 5 to 14 membered polycyclic heterocyclic alkyl group, wherein every two rings in the system share two disconnected atoms, the rings can have one or more double bonds, but have no completely conjugated pi-electron system, and the rings have one or more heteroatoms selected from the group consisting of N, O, and S(O)ₘ (wherein m is 0, 1, or 2) as ring atoms, the remaining ring atoms being C. Preferably a bridged heterocyclyl is 6 to 14 membered, and more preferably 7 to 10 membered. According to the number of membered rings, bridged heterocyclyl is divided into bicyclic, tricyclic, tetracyclic or polycyclic bridged heterocyclyl, and preferably refers to bicyclic, tricyclic or tetracyclic bridged heterocyclyl, more preferably bicyclic or tricyclic bridged heterocyclyl. Representative examples of bridged heterocyclyl include, but are not limited to, the following substituents:

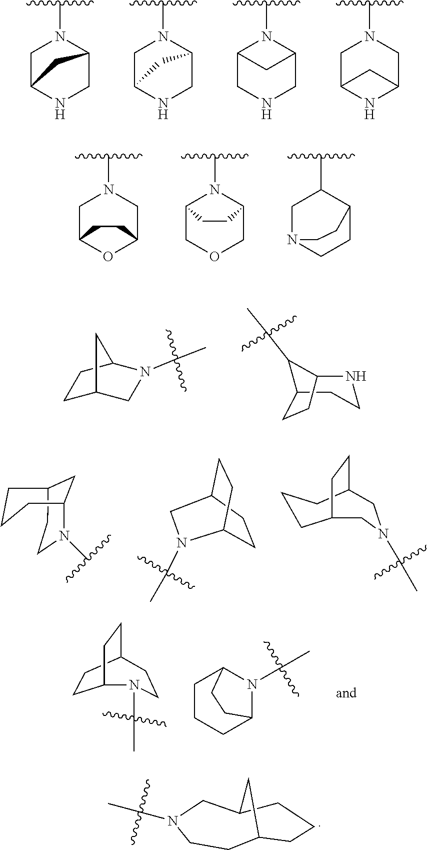

The ring of said heterocyclyl can be fused to the ring of an aryl, heteroaryl or cycloalkyl, wherein the ring bound to the parent structure is heterocyclyl. Representative examples include, but are not limited to the following substituents:

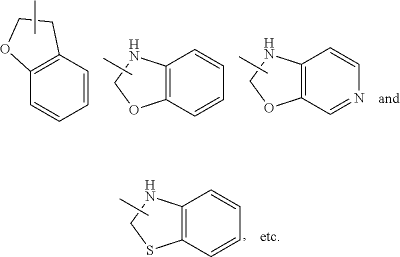

The heterocyclyl is optionally substituted or unsubstituted. When substituted, the substituent group(s) is preferably one or more, sometimes preferably one to five, sometimes more preferably one to three, group(s) independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio and —N⁹R¹⁰. “Aryl” refers to a 6 to 14 membered all-carbon monocyclic ring or a polycyclic fused ring (a “fused” ring system means that each ring in the system shares an adjacent pair of carbon atoms with another ring in the system) group, and has a completely conjugated pi-electron system. Preferably aryl is 6 to 10 membered, such as phenyl and naphthyl, most preferably phenyl. The aryl can be fused to the ring of heteroaryl, heterocyclyl or cycloalkyl, wherein the ring bound to parent structure is aryl. Representative examples include, but are not limited to, the following substituents:

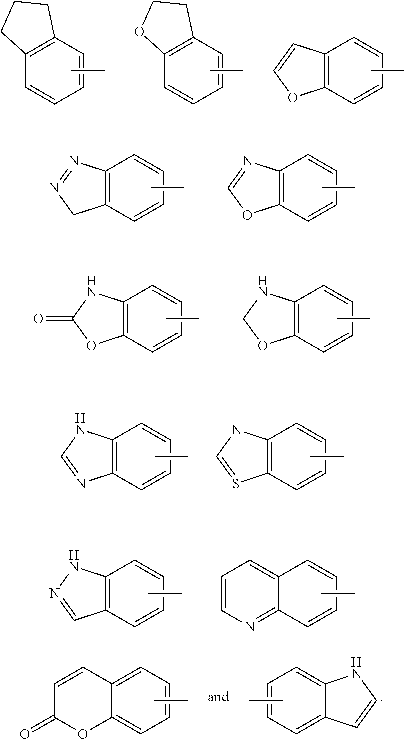

The aryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio.

“Heteroaryl” refers to an aryl system having 1 to 4 heteroatoms selected from the group consisting of O, S and N as ring atoms and having 5 to 14 annular atoms. Preferably a heteroaryl is 5- to 10-membered, more preferably 5- or 6-membered, for example, thiadiazolyl, pyrazolyl, oxazolyl, oxadiazolyl, imidazolyl, triazolyl, thiazolyl, furyl, thienyl, pyridyl, pyrrolyl, N-alkyl pyrrolyl, pyrimidinyl, pyrazinyl, imidazolyl, tetrazolyl, and the like. The heteroaryl can be fused with the ring of an aryl, heterocyclyl or cycloalkyl, wherein the ring bound to parent structure is heteroaryl. Representative examples include, but are not limited to, the following substituents:

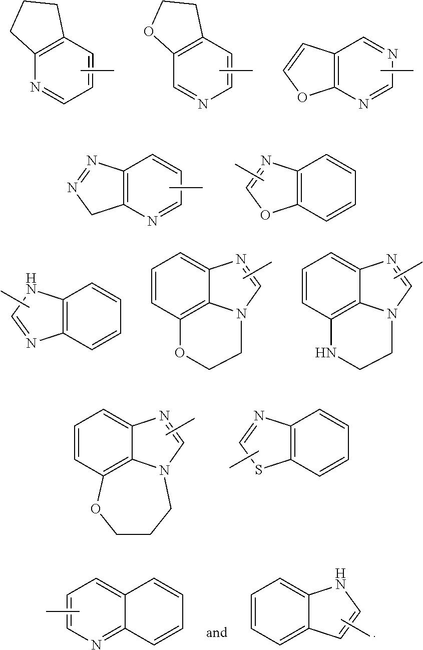

The heteroaryl group can be substituted or unsubstituted. When substituted, the substituent group(s) is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio, heterocylic alkylthio.

“Alkoxy” refers to both an —O-(alkyl) and an —O-(unsubstituted cycloalkyl) group, wherein the alkyl is defined as above. Representative examples include, but are not limited to, methoxy, ethoxy, propoxy, butoxy, cyclopropyloxy, cyclobutyloxy, cyclopentyloxy, cyclohexyloxy, and the like. The alkoxyl can be substituted or unsubstituted. When substituted, the substituent is preferably one or more, sometimes preferably one to five, sometimes more preferably one to three, substituents independently selected from the group consisting of alkyl, alkenyl, alkynyl, alkoxy, alkylsulfo, alkylamino, halogen, thiol, hydroxy, nitro, cyano, cycloalkyl, heterocyclic alkyl, aryl, heteroaryl, cycloalkoxyl, heterocylic alkoxyl, cycloalkylthio and heterocylic alkylthio.
“Bond” refers to a covalent bond using a sign of “—”.
“Hydroxyalkyl” refers to an alkyl group substituted by a hydroxy group, wherein alkyl is as defined above.
“Hydroxyl” or “hydroxy” refers to an —OH group.
“Halogen” or “halo” refers to fluoro, chloro, bromo or iodo.
“Amino” refers to a —NH₂ group.
“Cyano” refers to a —CN group.
“Nitro” refers to a —NO₂ group.
“Oxo group” refers to a ═0 group.
“Carboxyl” refers to a —C(O)OH group.
“Alkoxycarbonyl” refers to a —C(O)O(alkyl) or (cycloalkyl) group, wherein the alkyl and cycloalkyl are defined as above.

“Optional” or “optionally” means that the event or circumstance described subsequently can, but need not, occur, and the description includes the instances in which the event or circumstance may or may not occur. For example, “the heterocyclic group optionally substituted by an alkyl” means that an alkyl group can be, but need not be, present, and the description includes the case of the heterocyclic group being substituted with an alkyl and the heterocyclic group being not substituted with an alkyl.

“Substituted” refers to one or more hydrogen members in a group independently substituted with a corresponding number of substituents. In some embodiments, the number of such hydrogen members is up to 5. In other embodiments it si between 1 and 3. It goes without saying that the substituents exist in their only possible chemical position. The person skilled in the art is able to determine if the substitution is possible or impossible without paying excessive efforts by experiment or theory. For example, the combination of amino or hydroxyl group having free hydrogen and carbon atoms having unsaturated bonds (such as olefinic) may be unstable.

A “pharmaceutical composition” refers to a mixture of one or more of the compounds described in the present invention or physiologically/pharmaceutically acceptable salts or prodrugs thereof and other chemical components such as physiologically/pharmaceutically acceptable carriers and excipients. The purpose of a pharmaceutical composition is to facilitate administration of a compound to an organism, which is conducive to the absorption of the active ingredient and thus displaying biological activity.

“Pharmaceutically acceptable salts” refer to salts of the compounds described herein, such salts being safe and effective when used in a mammal and have corresponding biological activity.

One skilled in the art will recognize that in certain embodiments compounds described herein can have one or more asymmetric carbon atoms in their structure. As used herein, any chemical formulas with bonds shown only as solid lines and not as solid wedged or hashed wedged bonds contemplates each possible stereoisomer, or mixture of two or more stereoisomers. Stereoisomers includes enantiomers and diastereomers. Enantiomers are stereoisomers that are non-super-imposable mirror images of each other. A 1:1 mixture of a pair of enantiomers is a racemate or racemic mixture. Diastereomers (or diastereoisomers) are stereoisomers that are not enantiomers, i.e., they are not related as mirror images, and occur when two or more stereoisomers of a compound have different configurations at one or more of the equivalent stereocenters and are not mirror images of each other. Substituent groups (e.g., alkyl, heterocyclyl, etc.) can contain stereocenters in either the R or S configuration.

Thus, included within the scope of the invention are the stereochemically pure isomeric forms of the compounds described herein (i.e., a single enantiomer or a single diastereomer) as well as mixtures thereof including their racemates. For example, when a compound is for instance specified as (R), this means that the compound is substantially free of the (S) isomer. Compounds described herein can be used as racemic mixtures, enantiomerically or diastereomerically enriched mixtures, or as enantiomerically or diastereomerically pure individual stereoisomers.

Stereochemically pure isomeric forms can be obtained by techniques known in the art in view of the present disclosure. For example, diastereoisomers can be separated by physical separation methods such as fractional crystallization and chromatographic techniques, and enantiomers can be separated from each other by the selective crystallization of the diastereomeric salts with optically active acids or bases or by chiral chromatography. Pure stereoisomers can also be prepared synthetically from appropriate stereochemically pure starting materials, or by using stereoselective reactions.

Compounds described herein can also have mesomers. The term “mesomer” refers to a non-optically active stereoisomer. A mesomer contains two or more stereogenic centers but is not chiral.

Compounds described herein can also form tautomers. The term “tautomer” refers to compounds that are interchangeable forms of a particular compound structure and that vary in the displacement of hydrogen atoms and electrons. Tautomers are constitutional isomers of chemical compounds that readily interconvert, usually resulting in relocation of a proton (hydrogen). Thus, two structures can be in equilibrium through the movement of pi electrons and an atom (usually hydrogen). All tautomeric forms and mixtures of tautomers of the compounds described herein are included with the scope of the invention.

Compounds described herein can exist in solvated and unsolvated forms. The term “solvate” means a physical association, e.g., by hydrogen bonding, of a compound of the invention with one or more solvent molecules. The solvent molecules in the solvate can be present in a regular arrangement and/or a non-ordered arrangement. The solvate can comprise either a stoichiometric or nonstoichiometric amount of the solvent molecules. “Solvate” encompasses both solution-phase and isolable solvates. Compounds of the invention can form solvates with water (i.e., hydrates) or common organic solvents. Exemplary solvates include, but are not limited to, hydrates, ethanolates, methanolates, and isopropanolates.

As used herein, the name of a compound is intended to encompass all possible existing isomeric forms, including stereoisomers (e.g., enantiomers, diastereomers, racemate or racemic mixture, and any mixture thereof) of the compound.

### EXAMPLES

The following examples serve to illustrate the invention, but the examples should not be considered as limiting the scope of the invention. If specific conditions for an experimental method are not specified in the examples of the present invention, they are generally in accordance with conventional conditions or recommended conditions of the raw materials and the product manufacturer. The reagents without a specific source indicated are commercially available, conventional reagents.

The structure of each compound is identified by nuclear magnetic resonance (NMR) and/or mass spectrometry (MS). NMR chemical shifts (δ) are given in 10⁻⁶ (ppm). NMR is determined by Varian Mercury 300 MHz, Bruker Avance III 400 MHz machine. The solvents used are deuterated-dimethyl sulfoxide (DMSO-d₆), deuterated-chloroform (CDCl₃) and deuterated-methanol (CD₃OD).

High performance liquid chromatography (HPLC) is determined on an Agilent 1200DAD high pressure liquid chromatography spectrometer (Sunfire C18 150×4.6 mm chromatographic column) and a Waters 2695-2996 high pressure liquid chromatography spectrometer (Gimini C18 150×4.6 mm chromatographic column). Liquid Chromatography Mass Spectrometry (LCMS) is determined on an Agilent 1200 high pressure liquid chromatography spectrometer & mass spectrometry (Sunfire C18 4.6*50 mm 3.5 um chromatographic column) and an Agilent 19091S-433 HP-5 high pressure liquid chromatography spectrometer & mass spectrometry (XBridge C18 4.6*50 mm 3.5 um chromatographic column).

Chiral High performance liquid chromatography (HPLC) is determined on SFC Thar 80 & 150 & 200 (waters.)

The average rates of ATPase inhibition, and the IC₅₀ values are determined by Victor Nivo multimode plate reader (PerkinElmer, USA).

The thin-layer silica gel plates used in thin-layer chromatography are Yantai Xinnuo silica gel plate. The dimension of the plates used in TLC was 0.15 mm to 0.2 mm, and the dimension of the plates used in thin-layer chromatography for product purification is 0.4 mm to 0.5 mm.

Column chromatography generally uses Qingdao Haiyang 200 to 300 mesh silica gel as carrier.

The known starting material of the invention can be prepared by the conventional synthesis method in the prior art, or can be purchased from ABCR GmbH & Co. KG, Acros Organics, Aldrich Chemical Company, Accela ChemBio Inc or Dari chemical Company, etc.

Unless otherwise stated in the examples, the following reactions are performed under argon atmosphere or nitrogen atmosphere.

The term “argon atmosphere” or “nitrogen atmosphere” means that a reaction flask is equipped with a balloon having 1 L of argon or nitrogen.

The term “hydrogen atmosphere” means that a reaction flask is equipped with a balloon having 1 L of hydrogen.

MS is mass spectroscopy with (+) referring to the positive mode which generally gives a M+1 (or M+H) absorption where M=the molecular mass.

### General Procedure A

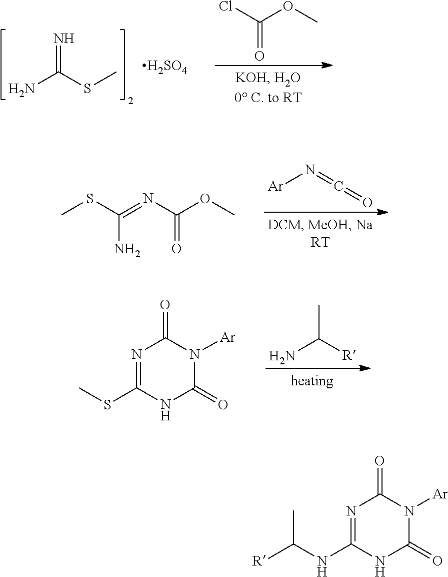

Methyl carbamimidothioate sulfuric acid salt is condensed with methyl chloroformate. The resulting carbamate underwent cycloaddition with commercial aryl isocyanate to give a six-membered triazine-dione core structure, which is then coupled with a commercially available or custom-made primary amine to give a triazine dione analoguevia nucleophilic addition under heating conditions.

### General Procedure B

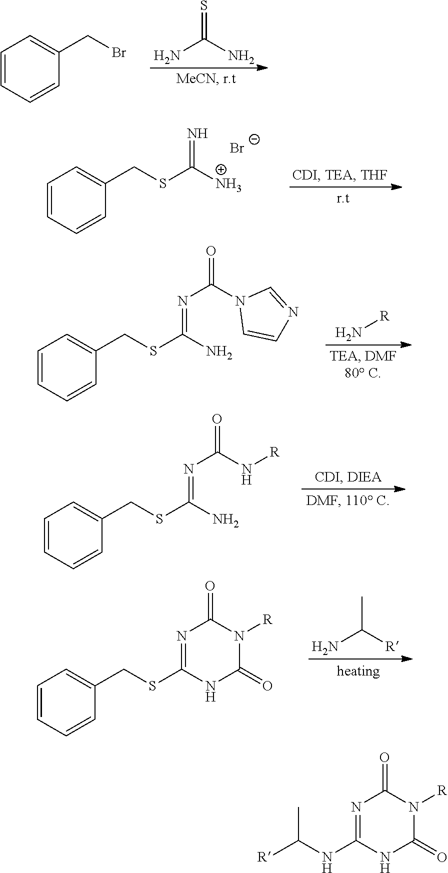

The condensation between benzyl bromide and thiourea offer a bromide salt, which is then condensed with carbonyl diimidazole to give a carbonyl mono-imidazole. Subsequent condensation with a commercially available or custom made amine lead to a urea that is subsequently cyclized under the catalysis of carbonyl diimidazole to give a six-membered core structure. Then six-membered core structure is subjected to nucleophilic substitution under heating conditions with a commercially available or custom-made primary amine to give a triazine dione analogue.

### General Procedure C

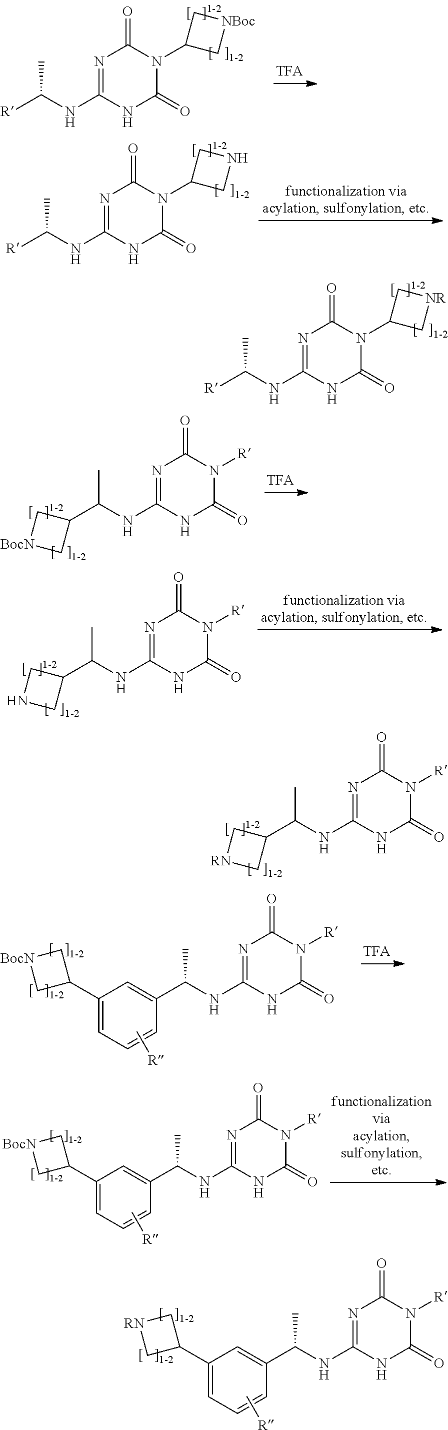

N-boc protected heterocycles or N-Boc substituted carbocycles are deprotected under typical acidic conditions, such as TFA or HCL. The resulting amines are either tested in biological assays or further functionalized via acylation or sulfonylation to give amide, carbamate, urea, or sulfonamide, etc.

### General Procedure D

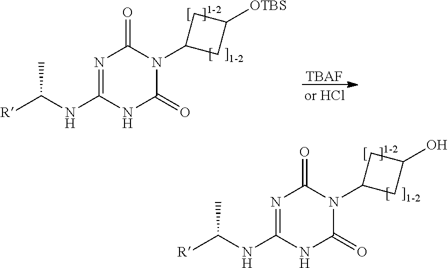

TBS protected alcohols are unmasked to give free alcohols under typical conditions, such as TBAF or IF-pyridine.

### General Procedure E

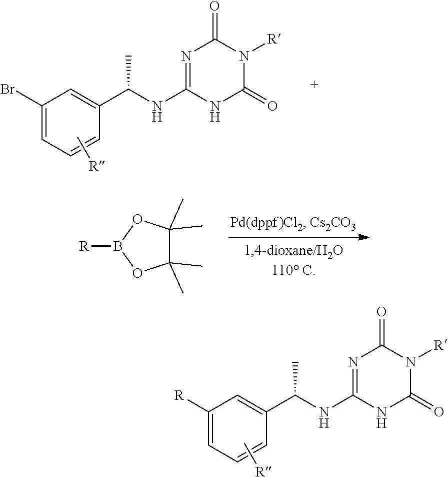

Aryl bromide are coupled with commercial aryl, vinyl, or alkyl boronic esters under typical Suzuki coupling conditions to give carbon-linked analogues.

### General Procedure F

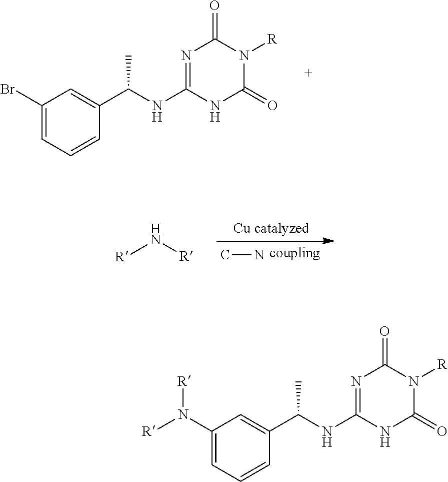

Aryl bromide are coupled with commercially available amines under typical Suzuki coupling conditions to give nitrogen-linked analogues.

### General Procedure G

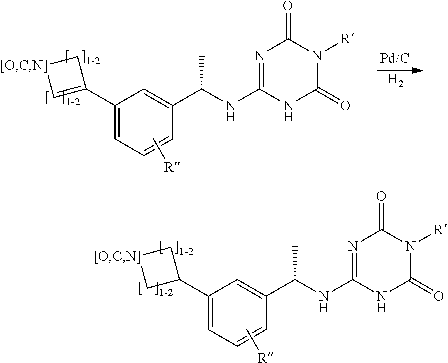

Olefins are reduced under typical hydrogenation conditions to give saturated heterocycles or carbocycles.

### EXAMPLES

The following examples are offered to illustrate but not limit to the compositions, uses, and the methods provided herein. The compounds were prepared using the general methods described above.

The following abbreviations are used throughout the examples: TEA (trimethylamine), DCM (dichloromethane), DMF (N,N-dimethylformamide), DIEA (diisopropylethylamine), MeOH (methanol), PE (petroleum ether), and EA (ethyl acetate).

### Example 1

### Step 1. Synthesis of Intermediate 1-1

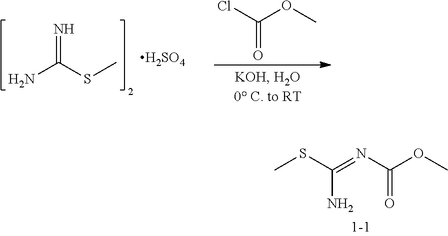

To a mixture of 1-methyl-2-thiopseudourea sulfate (13.9 g, 73.8 mmol) and methyl chloroformate (9.4 g, 99.4 mmol) in water (200 mL) at 0° C. was added dropwise a solution of KOH (11.38 g, 202.8 mmol) in water (40 mL). The reaction mixture was stirred at room temperature for 3 h and then extracted with DCM. The organic extracts were dried and the solvent was evaporated on a rotary evaporator to give intermediate 1-1 (9 g, 82.4%) as white solid.

ESI-MS (EI⁺, m/z): 149.10.

¹H NMR (400 MHz, Chloroform-d): δ 3.73 (s, 3H), 2.46 (s, 3H).

### Step 2. Synthesis of Intermediate 1-2

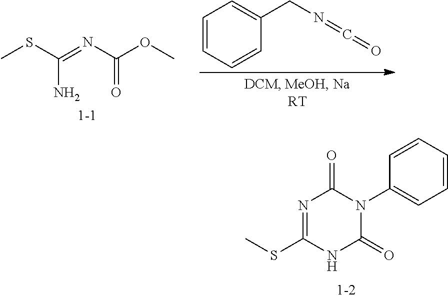

Intermediate 1-1 (1.0 g, 6.75 mmol) was dissolved in DCM (10 mL). Isocyanatobenzene (804 mg, 6.75 mmol) was added to the solution over 5 min and the mixture was stirred at room temperature for 2.5 h. A freshly prepared solution of sodium (155 mg, 6.75 mmol) in MeOH (1.3 mL) was then added over 5 min and the resulting mixture was stirred at room temperature for 16 h. The mixture was concentrated and the residue was dissolved in water. The aqueous solution was extracted with ethyl acetate (20 mL *2) to remove neutral byproducts, and then acidified with concentrated HCl to pH 1. The precipitated solid was separated by filtration, washed with water and dried to give intermediate 1-2 (660 mg, 41.7%) as white solid.

ESI-MS (EI⁺, m/z): 236.10

¹H NMR (400 MHz, Methanol-d₄): δ 7.52-7.39 (m, 3H), 7.32-7.26 (m, 2H), 2.61 (s, 3H).

### Step 3. Synthesis of Example 1

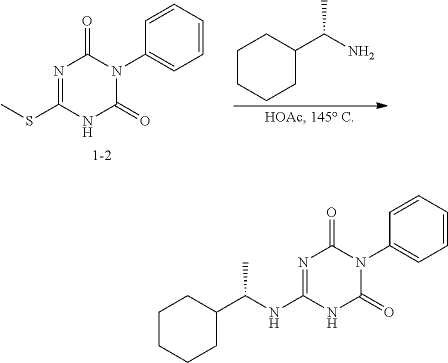

A microwave vial was charged with (S)-1-cyclohexylethan-1-amine (106 mg, 0.84 mmol) and HOAc (1.0 mL), and the resulting mixture was stirred at room temperature for 0.5 h, then intermediate 1-2 (100 mg, 0.42 mmol) was added, the vial was sealed and the resulting mixture was heated to 145° C. for 4 h. The mixture was cooled to room temperature, water was added, and the mixture was stirred at room temperature for 15 min. The mixture was filtered, and the filtrate cake was washed with water and dried to afford the title compound (85 mg, 64.3%) as white solid.

ESI-MS (EI⁺, m/z): 315.25.

¹H NMR (400 MHz, DMSO-d₆) δ 7.47-7.30 (m, 3H), 7.22 (dd, J=7.2, 1.8 Hz, 2H), 6.82 (br, 1H), 3.79-3.86 (m, 1H), 1.78-1.59 (m, 5H), 1.47-1.34 (m, 1H), 1.27-1.12 (m, 3H), 1.10 (d, J=6.7 Hz, 3H), 1.04-0.91 (m, 2H).

### Example 2

### Step 1. Synthesis of Intermediate 2-1

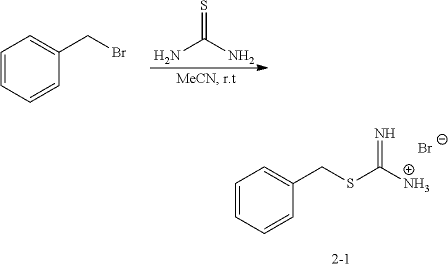

A solution of (bromomethyl)benzene (10.0 g, 58.8 mmol) in CH₃CN (100 mL) was added thiourea (6.0 g, 78.9 mmoL, 1.3 eq.). The resulting mixture was stirred at room temperature for 3 h. The reaction solution was filtered and washed with CH₃CN (50 mL), the filtrate cake was dried under vacuumto afford intermediate 2-1 (13.0 g, 90.2%) as white solid.

¹H NMR (400 MHz, DMSO-d₆) δ 9.06 (s, 4H), 7.45-7.30 (m, 5H), 4.48 (s, 2H).

### Step 2. Synthesis of Intermediate 2-2

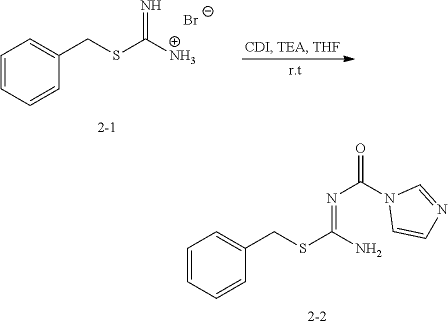 [SMILES: N=C([NH3+])SCc1ccccc1 ; [Br-] ; N/C(=N\C(=O)n1ccnc1)SCc1ccccc1]

A solution of intermediate 2-1 (10.0 g, 40.6 mmol) in THE (100 mL) was added CDI (8.8 g, 54.2 mmoL, 1.3 eq.) and Et₃N (5.4 g, 54.2 mmol, 1.3 eq.). The resulting mixture was stirred at room temperature under N₂ for 2 h until TLC showed the reaction was completed. The reaction solution was filtered and the filtrate was concentrated under vacuum. The residue was purified with silica gel column (DCM:MeOH=30:1) to afford the intermediate 2-2 (7.0 g, 67.3%) as white solid.

ESI-MS (EI⁺, m/z): 261.15.

¹H NMR (400 MHz, DMSO-d₆) δ 9.36 (d, J=59.7 Hz, 2H), 8.33-8.25 (m, 1H), 7.61 (t, J=1.3 Hz, 1H), 7.44-7.24 (m, 5H), 7.01-6.95 (m, 1H), 4.43 (s, 2H).

### Step 3. Synthesis of Intermediate 2-3

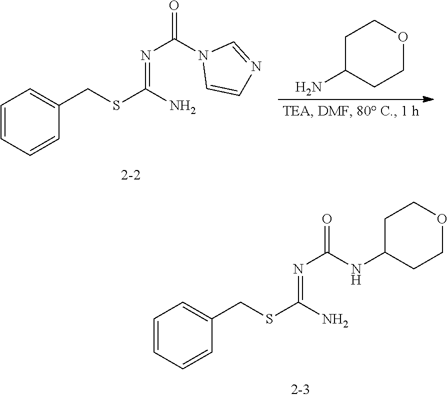 [SMILES: N/C(=N\C(=O)n1ccnc1)SCc1ccccc1 ; NC1CCOCC1 ; N/C(=N\C(=O)NC1CCOCC1)SCc1ccccc1]

A solution of intermediate 2-2 (3.0 g, 11.5 mmol) in DMF (10 mL) was added tetrahydro-2H-pyran-4-amine (1.75 g, 17.3 mmol, 1.5 eq.) and Et₃N (2.3 g, 23.0 mmol, 2.0 eq.). The resulting mixture was stirred at 80° C. under N₂ for 1 h until TLC and LCMS showed the reaction was completed. The reaction solution was diluted with water and extracted twice with EtOAc, The organics were washed with water and brine, dried over Na₂SO₄, filtered and concentrated under vacuum. The reaction mixture was purified with silica gel column (DCM:MeOH=30:1) to afford intermediate 2-3 (1.5 g, 44.6%) as yellow solid.

ESI-MS (EI⁺, m/z): 294.20.

¹H NMR (400 MHz, DMSO-d₆) δ 8.49 (s, 2H), 7.41-7.34 (m, 2H), 7.30 (t, J=7.4 Hz, 2H), 7.23 (dd, J=8.3, 6.1 Hz, 1H), 7.07 (d, J=8.0 Hz, 1H), 4.28 (s, 2H), 3.87-3.76 (m, 2H), 3.60 (ddt, J=15.0, 7.7, 4.4 Hz, 1H), 3.35 (d, J=1.7 Hz, 1H), 3.29 (d, J=1.8 Hz, 1H), 1.73-1.62 (m, 2H), 1.45 (qd, J=12.1, 4.4 Hz, 2H).

### Step 4. Synthesis of Intermediate 2-4

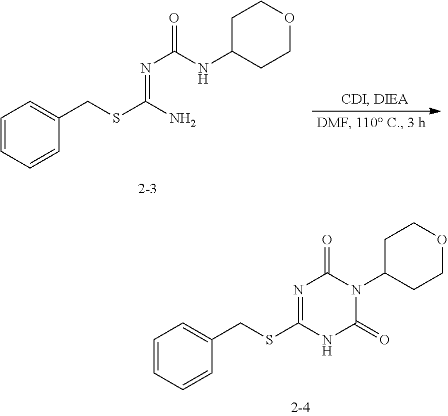 [SMILES: N/C(=N\C(=O)NC1CCOCC1)SCc1ccccc1 ; O=c1nc(SCc2ccccc2)[nH]c(=O)n1C1CCOCC1]

A solution of intermediate 2-3 (4.5 g, 15.3 mmol) in DMF (15 mL) was added CDI (4.8 g, 29.6 mmol, 2.0 eq.) and DIEA (3.9 g, 30.2 mmol, 2.0 eq.). The resulting mixture was stirred at 110° C. for 3 h until TLC and LCMS showed the reaction was completed. The reaction mixture was purified with reversed-phase column to afford the compound intermediate 2-4 (1.0 g, 21.2%) as yellow liquid.

ESI-MS (EI⁺, m/z): 320.15.

¹H NMR (400 MHz, DMSO-d₆) δ 7.40-7.35 (m, 2H), 7.30 (t, J=7.4 Hz, 2H), 7.23 (dd, J=8.4, 6.1 Hz, 1H), 4.24 (s, 2H), 3.89 (dd, J=11.1, 4.1 Hz, 2H), 3.64-3.56 (m, 1H), 3.30 (t, J=11.2 Hz, 2H), 2.59 (qd, J=12.4, 4.7 Hz, 2H), 1.40-1.33 (m, 2H).

### Step 5. Synthesis of Example 2

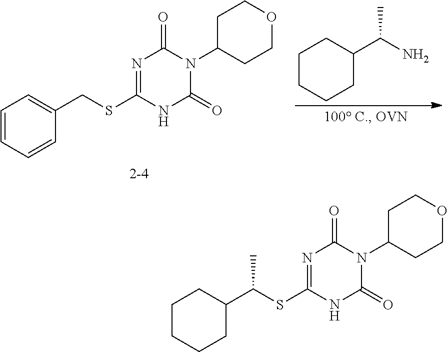

A solution of intermediate 2-4 (300 mg, 0.94 mmol) in (S)-1-cyclohexylethan-1-amine (300 mg, 2.36 mmol) was stirred at 90° C. in a sealed tube overnight until LCMS showed the reaction was completed. The reaction mixture was purified with prep-HPLC to afford the title compound (45 mg, 14.9%) as white solid.

ESI-MS (EI⁺, m/z): 323.23.

¹H NMR (400 MHz, DMSO-d₆) δ 10.32 (s, 1H), 6.61 (d, J=4.8 Hz, 1H), 4.66 (t, J=12.1 Hz, 1H), 3.89 (dd, J=11.2, 4.2 Hz, 2H), 3.75 (d, J=5.0 Hz, 1H), 3.33 (s, 1H), 3.28 (s, 1H), 2.53 (s, 1H), 2.45 (dd, J=12.6, 4.6 Hz, 1H), 1.76-1.56 (m, 5H), 1.42 (d, J=12.5 Hz, 3H), 1.24-1.10 (m, 3H), 1.05 (d, J=6.7 Hz, 3H), 0.99-0.86 (m, 2H).

### Example 3

### Step 1. Synthesis of Intermediate 3-1

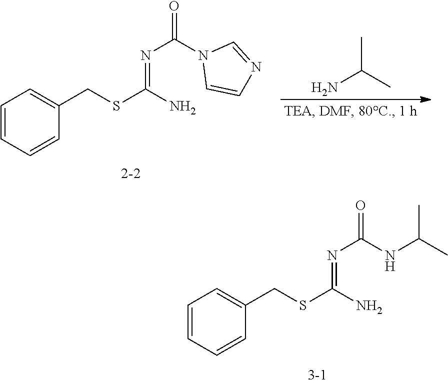

A solution of intermediate 2-2 (3.0 g, 11.5 mmol) in DMF (10 mL) was added propan-2-amine (1.0 g, 17.3 mmol, 1.5 eq.) and Et₃N (2.3 g, 23.0 mmol, 2.0 eq.). The resulting mixture was stirred at 80° C. under N₂ for 1 h until TLC and LCMS showed the reaction was completed. The reaction solution was diluted with water and extracted twice with EtOAc. The organic layer was washed with water and brine, dried over Na₂SO₄, filtered and concentrated under vacuum. The residue was purified with silica gel column (DCM:MeOH=30:1) to afford intermediate 3-1 (800.0 mg, 28.5%) as yellow solid. ESI-MS (EI⁺, m/z): 252.15.

¹H NMR (400 MHz, Chloroform-d) δ 7.33-7.17 (m, 5H), 4.20 (s, 2H), 3.86 (dq, J=14.0, 6.6 Hz, 1H), 1.13 (d, J=6.5 Hz, 6H).

### Step 2. Synthesis of Intermediate 3-2

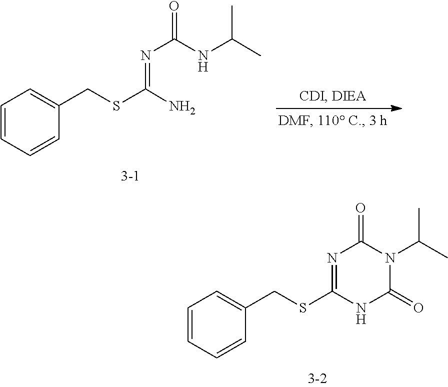 [SMILES: CC(C)NC(=O)/N=C(\N)SCc1ccccc1 ; CC(C)n1c(=O)nc(SCc2ccccc2)[nH]c1=O]

A solution of intermediate 3-1 (800.0 mg, 3.2 mmol) in DMF (5 mL) was added CDI (1.1 g, 6.4 mmol, 2.0 eq.) and DIEA (823.0 g, 6.4 mmol, 2.0 eq.). The resulting mixture was stirred at 110° C. for 3 h until TLC and LCMS showed the reaction was completed. The reaction mixture was purified with reversed-phase column to afford intermediate 3-2 (200.0 mg, 24.3%) as yellow liquid.

ESI-MS (EI⁺, m/z): 278.20.

¹H NMR (400 MHz, DMSO-d₆) δ 7.42-7.35 (m, 2H), 7.35-7.28 (m, 2H), 7.28-7.21 (m, 2H), 4.86 (p, J=6.9 Hz, 1H), 4.28 (s, 2H), 1.32 (d, J=6.9 Hz, 6H).

### Step 3. Synthesis of Example 3

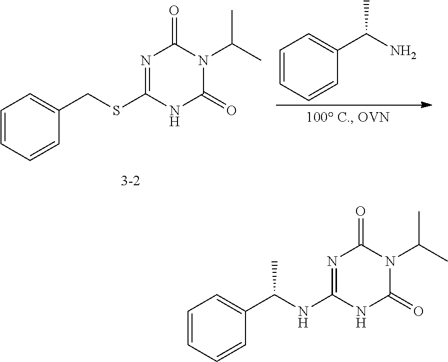

A solution of intermediate 3-2 (150 mg, 0.541 mmol) in (S)-1-phenylethan-1-amine (656 mg, 5.409 mmol) was stirred at 100° C. in a sealed tube overnight until LCMS showed the reaction was completed. The reaction mixture was purified with reversed-phase column (˜40% MeCN, 0.1% Formate) to afford the title compound (5.8 mg, 3.9%) as white solid.

ESI-MS (EI⁺, m/z): 275.25.

¹H NMR (400 MHz, DMSO-d₆) δ 10.46 (s, 1H), 7.35 (d, J=4.8 Hz, 4H), 7.28-7.24 (m, 1H), 5.04 (p, J=7.0 Hz, 1H), 4.80 (hept, J=6.8 Hz, 1H), 1.42 (d, J=6.9 Hz, 3H), 1.30 (d, J=6.9 Hz, 6H).

### Example 4

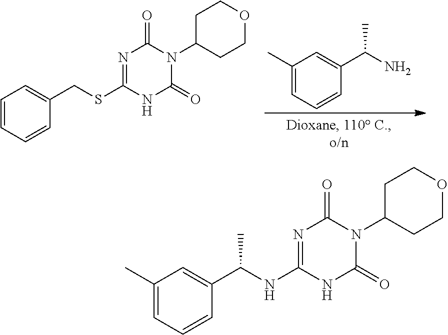

A 20.0 mL microwave tube was equipped with 6-(benzylthio)-3-(tetrahydro-2H-pyran-4-yl)-1,3,5-triazine-2,4(1H,3H)-dione (200 mg, 0.063 mmol), (S)-1-(m-tolyl)ethan-1-amine (127 mg, 0.094 mmol) in dioxane (5.0 mL) and heated to 110° C. The resulting solution was concentrated to dryness under vacuum. The crude was purified by prep-HPLC to give the title compound (101.4 mg, yield: 49.0%).

MS: m/z=331.1 (M+1, ESI⁺).

¹H NMR (400 MHz, MeOD) δ 7.15 (ddd, J=34.6, 21.0, 7.6 Hz, 4H), 5.10 (q, J=6.8 Hz, 1H), 4.80 (tt, J=12.2, 4.0 Hz, 1H), 3.99 (dd, J=11.4, 3.8 Hz, 2H), 3.44 (t, J=11.7 Hz, 2H), 2.66 (qd, J=12.4, 4.8 Hz, 2H), 1.51 (t, J=11.8 Hz, 5H).

### Example 5

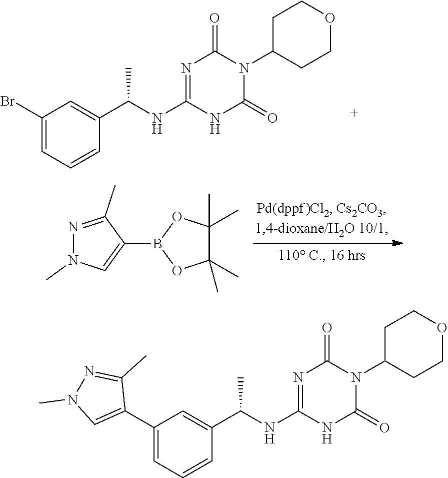

To a solution of (S)-6-((1-(3-bromophenyl)ethyl)amino)-3-(tetrahydro-2H-pyran-4-yl)-1,3,5-triazine-2,4(1H,3H)-dione (100 mg, 0.25 mmol) (prepared in an analogous fashion from (S)-1-(3-bromophenyl)ethan-1-amine following the synthetic procedure of Example 4) and 1,3-dimethyl-4-(4,4,5,5-tetramethyl-1,3,2-dioxaborolan-2-yl)-1H-pyrazole (83 mg, 0.375 mmol) in 1,4-dioxane (5.00 mL) and H₂O (0.5 mL) was added Pd(dppf)Cl₂ (18 mg, 0.25 mmol) and Cs₂CO₃ (165 mg, 0.5 mmol). The mixture was stirred at 110° C. for 16 h under N₂. The solvent was removed under vacuum. The residue was diluted with water (10 mL) and extracted with DCM (10 mL×3). The combined organic layers were dried over Na₂SO₄, filtered, and concentrated. The residue was purified by prep-HPLC to provide the title compound (35.7 mg, 34% yield) as white solid.

MS: m/z=411.1 (M+1, ESI⁺).

¹H NMR (500 MHz, DMSO) δ 7.86 (s, 1H), 7.36 (dd, J=13.3, 5.5 Hz, 2H), 7.29 (d, J=7.8 Hz, 1H), 7.20 (d, J=7.6 Hz, 1H), 5.14-5.00 (m, 1H), 4.72-4.59 (m, 1H), 3.87 (d, J=11.1 Hz, 2H), 3.77 (s, 3H), 3.28 (d, J=11.9 Hz, 2H), 2.49-2.42 (m, 2H), 2.27 (s, 3H), 1.45 (d, J=6.9 Hz, 3H), 1.39 (d, J=10.6 Hz, 2H).

### Example 6

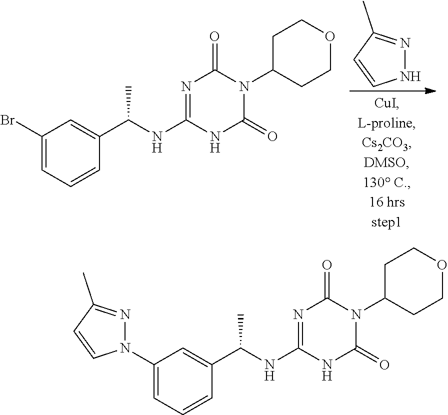

To a solution of (S)-6-((1-(3-bromophenyl)ethyl)amino)-3-(tetrahydro-2H-pyran-4-yl)-1,3,5-triazine-2,4(1H,3H)-dione (100 mg, 0.3 mmol) (prepared in an analogous fashion from (S)-1-(3-bromophenyl)ethan-1-amine following the synthetic procedure of Example 4) and SM1 (42 mg, 0.5 mmol) in DMSO (5.00 mL) was added CuI (72 mg, 0.4 mmol), L-proline (43 mg, 0.4 mmol) and Cs₂CO3 (248 mg, 0.8 mmol). The mixture was stirred at 130° C. for 16 h under N₂. The mixture was filtered and purified by prep-HPLC to provide the title compound (12.4 mg, 12% yield) as white solid.

MS: m/z=397.1 (M+1, ESI⁺).

1H NMR (400 MHz, MeOD) δ 8.10 (s, 1H), 7.72 (s, 1H), 7.57 (d, J=8.0 Hz, 1H), 7.45 (t, J=7.9 Hz, 1H), 7.31 (d, J=7.6 Hz, 1H), 6.32 (s, 1H), 5.23 (d, J=6.8 Hz, 1H), 4.78 (t, J=12.0 Hz, 1H), 3.99 (dd, J=11.6, 3.8 Hz, 2H), 3.44 (t, J=11.9 Hz, 2H), 2.74-2.55 (m, 2H), 2.33 (s, 3H), 1.55 (dd, J=18.3, 9.5 Hz, 5H).

### Example 7

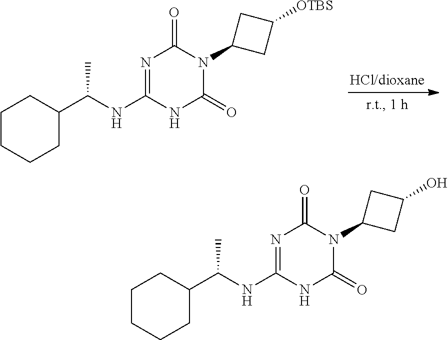 [SMILES: C[C@H](Nc1nc(=O)n([C@H]2C[C@H](O[Si](C)(C)C(C)(C)C)C2)c(=O)[nH]1)C1CCCCC1 ; C[C@H](Nc1nc(=O)n([C@H]2C[C@H](O)C2)c(=O)[nH]1)C1CCCCC1]

A solution of 3-((1r,3S)-3-((tert-butyldimethylsilyl)oxy)cyclobutyl)-6-(((S)-1-cyclohexylethyl)amino)-1,3,5-triazine-2,4(1H,3H)-dione (110 mg, 0.26 mmol) (prepared in an analogous fashion from (1r,3r)-3-((tert-butyldimethylsilyl)oxy)cyclobutan-1-amine and (S)-1-cyclohexylethan-1-amine following the synthetic procedure of Example 2 and 4) in HCl/dioxane (2 mL, 1.0N, 2.0 mmol) was stirred at rt for 3 h. The solution was purified by pre-HPLC to give title compound (5.0 mg, 6% yield) as white solid.

MS: m/z=309 (M+H, ESI⁺).

¹H NMR (400 MHz, CD3OD) δ5.50-5.39 (m, 1H), 4.54 (s, 1H), 3.92-3.84 (m, 1H), 3.06 (ddd, J=15.0, 10.5, 7.6 Hz, 2H), 2.23 (ddd, J=13.5, 7.3, 1.9 Hz, 2H), 1.87-1.60 (m, 6H), 1.41 (s, 1H), 1.31-1.18 (m, 3H), 1.14 (d, J=6.7 Hz, 3H), 1.10-0.89 (m, 3H).

### Example 8

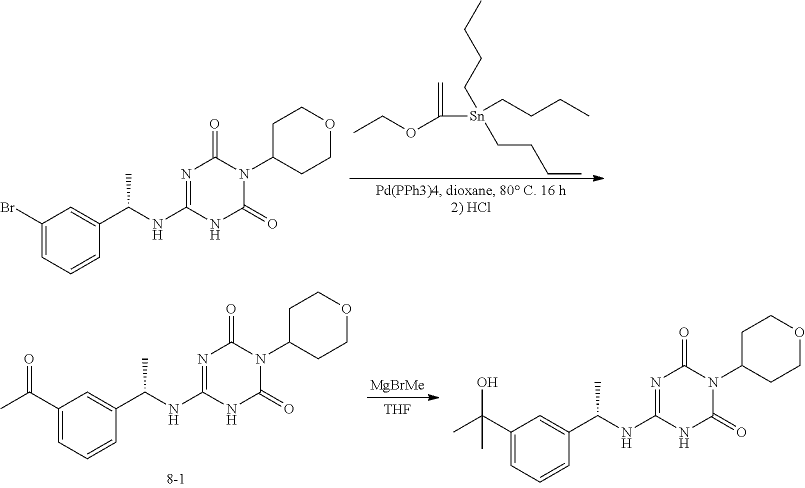

### Step 1. Synthesis of Intermediate 8-1

To a solution of (S)-6-((1-(3-bromophenyl)ethyl)amino)-3-(tetrahydro-2H-pyran-4-yl)-1,3,5-triazine-2,4(1H,3H)-dione (300 mg, 0.8 mmol) (prepared in an analogous fashion from (S)-1-(3-bromophenyl)ethan-1-amine following the synthetic procedure of Example 4) and tributyl(1-ethoxyvinyl)stannane (551 mg, 1.5 mmol) in 1,4-dioxane (20.00 mL) was added Pd(pph₃)₄ (175 mg, 0.2 mmol), and the resulting mixture was stirred at 80° C. for 16 h. The mixture was filtered and concentrated. The residue was dissolved in 1N HCl (1 mL) and THE (3 mL) and stirred at room temperature for 1 h. The mixture was concentrated and purified by flash chromatography (SiO₂, 10/1 DCM/MeOH) to provide the intermediate 8-1 (110 mg, 42% yield) as yellow oil.

MS: m/z=359.1 (M+1, ESI⁺).

### Step 2. Synthesis of Example 8

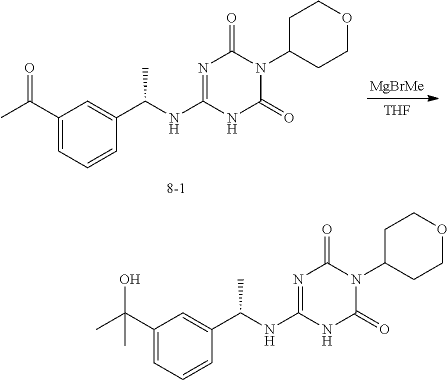 [SMILES: CC(=O)c1cccc([C@H](C)Nc2nc(=O)n(C3CCOCC3)c(=O)[nH]2)c1 ; C[C@H](Nc1nc(=O)n(C2CCOCC2)c(=O)[nH]1)c1cccc(C(C)(C)O)c1 ; "MgBrMe" ; "THF"]

A solution of intermediate 8-1 (100 mg, 0.3 mmol) in THE (3.00 mL) was stirred at 0° C., and MgBrMe (1.1 mL) was added. The mixture was stirred at 0° C. for 3 h. The mixture was quenched by H₂O (1 mL) and concentrated. The residue was purified by prep-HPLC to provide the title compound (12.4 mg, 12% yield) as white solid.

MS: m/z=357.2 (M+1, ESI⁺).

¹H NMR (400 MHz, MeOD) δ 7.50 (d, J=8.3 Hz, 1H), 7.38 (d, J=7.8 Hz, 1H), 7.30 (t, J=7.7 Hz, 1H), 7.22 (d, J=7.6 Hz, 1H), 5.15 (q, J=6.9 Hz, 1H), 4.79 (ddd, J=16.0, 8.1, 3.9 Hz, 1H), 4.00 (dd, J=11.3, 4.1 Hz, 2H), 3.44 (t, J=11.8 Hz, 2H), 2.63 (dt, J=12.6, 5.5 Hz, 2H), 1.59-1.44 (m, 11H).

### Example 9

### Step 1. Synthesis of Intermediate 9-1

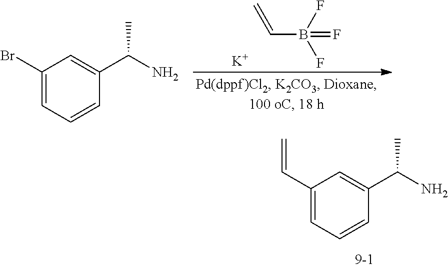

To a solution of (S)-1-(3-bromophenyl)ethan-1-amine (1.5 g, 7.5 mmol) and potassium vinyltrifluoroborate (2.0 g, 15.1 mmol) in 1,4-dioxane (20.00 mL) and H₂O (2 mL) was added Pd(dppf)Cl₂ (1.1 g, 1.5 mmol) and K₂CO₃ (3.1 g, 22.6 mmol). The mixture was stirred at 110° C. for 2.0 h under N₂. The residue was diluted with water (20 mL) and extracted with DCM (20 mL×3). The organic layers were combined and dried over Na₂SO₄, filtered, and concentrated. The residue was purified by flash chromatography (SiO₂, 10/1 DCM/MeOH) to provide intermediate 9-1 (830 mg, 75% yield) as yellow oil. MS: m/z=148.1 (M+1, ESI⁺).

### Step 2. Synthesis of Intermediate 9-2

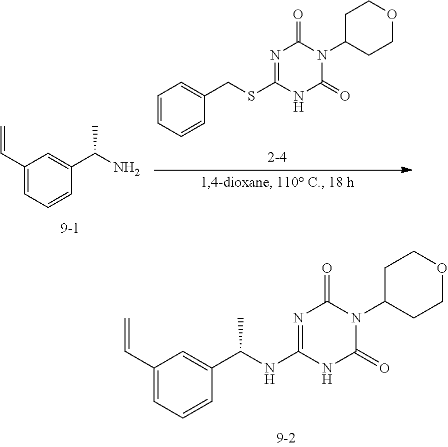 [SMILES: C=Cc1cccc([C@H](C)N)c1 ; O=c1nc(SCc2ccccc2)[nH]c(=O)n1C1CCOCC1 ; C=Cc1cccc([C@H](C)Nc2nc(=O)n(C3CCOCC3)c(=O)[nH]2)c1]

A solution of intermediate 9-1 (800 mg, 7.5 mmol) and intermediate 2-4 (1.4 g, 15.1 mmol) in 1,4-dioxane (20.00 mL) was stirred at 110° C. for 16.0 h. The residue was concentrated under vacuum and purified by flash chromatography (SiO₂, 1/1 PE/EA) to provide intermediate 9-2 (1.2 g, 69% yield) as yellow oil.

MS: m/z=148.1 (M+1, ESI⁺).

### Step 3. Synthesis of Intermediate 9-3

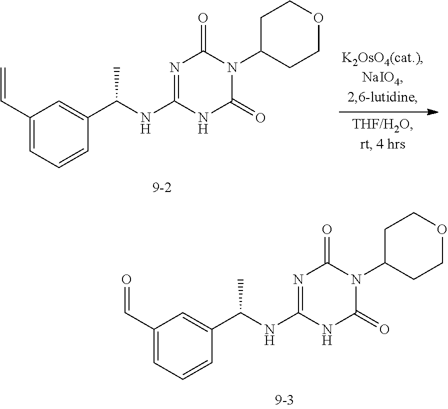 [SMILES: C=Cc1cccc([C@H](C)Nc2nc(=O)n(C3CCOCC3)c(=O)[nH]2)c1 ; C[C@H](Nc1nc(=O)n(C2CCOCC2)c(=O)[nH]1)c1cccc(C=O)c1]

To a solution of intermediate 9-2 (1.1 g, 3.2 mmol) and 2,6-lutidine (344 mg, 3.2 mmol) in THE (20.00 mL) and H₂O (4.00 mL) was added NaIO₄ (2.75 g, 12.8 mmol) and K₂OsO₄·2H₂O (118 mg, 0.3 mmol), and the resulting mixture was stirred at 25° C. for 4.0 h. The mixture was filtered and concentrated. The residue was purified by flash chromatography (SiO₂, 10/1 DCM/MeOH) to provide intermediate 9-3 (600 mg, 55% yield) as yellow oil.

MS: m/z=148.1 (M+1, ESI⁺).

### Step 4. Synthesis of Example 9

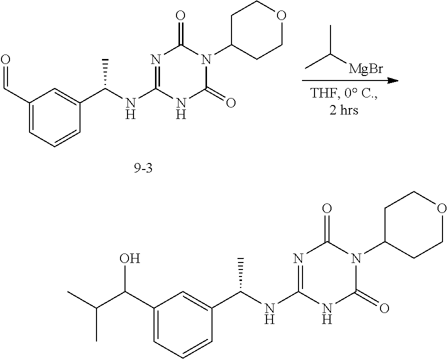

A mixture of intermediate 9-3 (80 mg, 0.2 mmol) in THE (3.00 mL) was stirred at 0° C., isopropyl magnesium bromide (0.9 mL) was added, and the resulting mixture was stirred at 0° C. for 3.0 h. The mixture was quenched with H₂O (1 mL) and concentrated. The residue was purified by prep-HPLC to provide the title compound (18.3 mg, 22% yield) as white solid.

MS: m/z=389.2 (M+1, ESI⁺).

¹H NMR (400 MHz, MeOD) δ 7.39-7.10 (m, 4H), 5.15 (q, J=6.9 Hz, 1H), 4.79 (ddd, J=12.1, 8.2, 4.2 Hz, 1H), 4.29 (dd, J=6.9, 2.7 Hz, 1H), 4.00 (dd, J=11.4, 4.3 Hz, 2H), 3.44 (t, J=11.7 Hz, 2H), 2.65 (qd, J=12.3, 4.6 Hz, 2H), 1.90 (dq, J=13.6, 6.9 Hz, 1H), 1.67-1.36 (m, 5H), 0.96 (dd, J=6.7, 3.9 Hz, 3H), 0.76 (dd, J=6.8, 1.4 Hz, 3H).

### Example 10

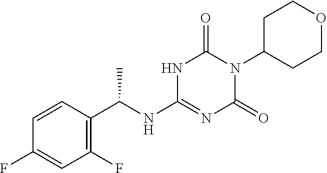 [SMILES: C[C@H](Nc1nc(=O)n(C2CCOCC2)c(=O)[nH]1)c1ccc(F)cc1F]

Example 10 was prepared from (S)-1-(2,4-difluorophenyl)ethan-1-amine in the same manner as Example 4.

MS: m/z=353.4 (M+1, ESI⁺).

¹HNMR(500 MHz, MeOD) δ 7.51-7.32 (m, 1H), 6.95 (ddt, J=13.8, 8.4, 2.6 Hz, 2H), 5.33 (q, J=7.0 Hz, 1H), 4.79 (tt, J=12.2, 4.0 Hz, 1H), 3.99 (dd, J=11.6, 3.8 Hz, 2H), 3.44 (t, J=12.0 Hz, 2H), 2.74-2.57 (m, 2H), 1.52 (t, J=5.8 Hz, 5H).

The compounds in the table below (Table 1) were prepared by similarly following the procedures described above.

TABLE 1

| Example | | Reference synthetic | |
|---|---|---|---|
| number | Structure | procedure | ESI (M + 1) |
| 11 | 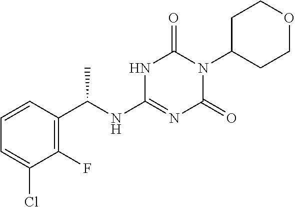 [SMILES: C[C@H](Nc1nc(=O)n(C2CCOCC2)c(=O)[nH]1)c1cccc(Cl)c1F] | general procedure B Example 2 | 369.1 |
| 12 | 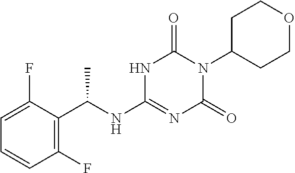 [SMILES: C[C@H](Nc1nc(=O)n(C2CCOCC2)c(=O)[nH]1)c1c(F)cccc1F] | general procedure B Example 2 | 353.1 |
| 13 | 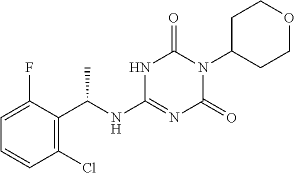 [SMILES: C[C@H](Nc1nc(=O)n(C2CCOCC2)c(=O)[nH]1)c1c(F)cccc1Cl] | general procedure B Example 2 | 369.1 |
| 14 | 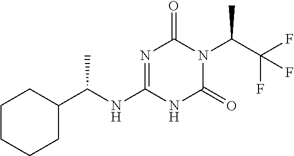 [SMILES: C[C@H](Nc1nc(=O)n([C@@H](C)C(F)(F)F)c(=O)[nH]1)C1CCCCC1] | general procedure B Example 2 | 335.2 |
| 15 | 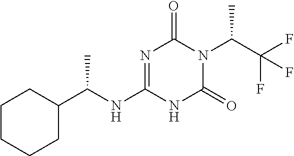 [SMILES: C[C@H](Nc1nc(=O)n([C@H](C)C(F)(F)F)c(=O)[nH]1)C1CCCCC1] | general procedure B Example 2 | 335.3 |
| 16 | 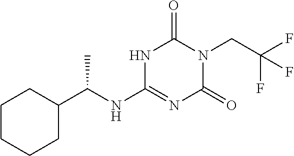 [SMILES: C[C@H](Nc1nc(=O)n(CC(F)(F)F)c(=O)[nH]1)C1CCCCC1] | general procedure B Example 2 | 321.3 |
| 17 | 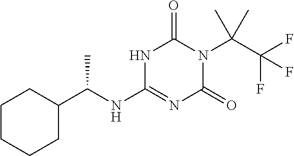 [SMILES: C[C@H](Nc1nc(=O)n(C(C)(C)C(F)(F)F)c(=O)[nH]1)C1CCCCC1] | general procedure B Example 2 | 349.4 |
| 18 | 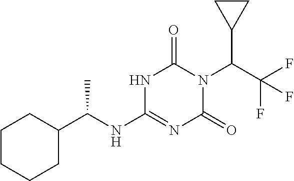 [SMILES: C[C@H](Nc1nc(=O)n(C(C2CC2)C(F)(F)F)c(=O)[nH]1)C1CCCCC1] | general procedure B Example 2 | 361.4 |
| 19 | 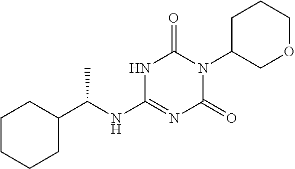 [SMILES: C[C@H](Nc1nc(=O)n(C2CCCOC2)c(=O)[nH]1)C1CCCCC1] | general procedure B Example 2 | 323.2 |
| 20 | 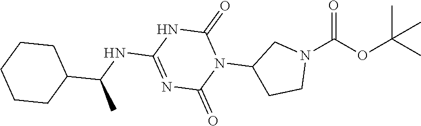 [SMILES: C[C@H](Nc1nc(=O)n(C2CCN(C(=O)OC(C)(C)C)C2)c(=O)[nH]1)C1CCCCC1] | general procedure B Example 2 | 408.5 |
| 21 | 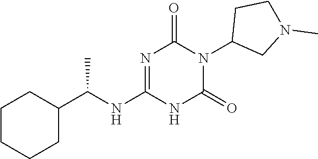 [SMILES: C[C@H](Nc1nc(=O)n(C2CCN(C)C2)c(=O)[nH]1)C1CCCCC1] | general procedure B Example 2 | 322.4 |
| 22 | 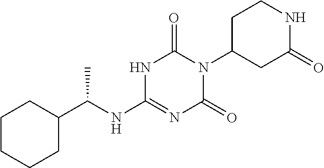 [SMILES: C[C@H](Nc1nc(=O)n(C2CCNC(=O)C2)c(=O)[nH]1)C1CCCCC1] | general procedure B Example 2 | 336.4 |
| 23 | 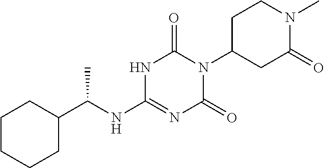 [SMILES: C[C@H](Nc1nc(=O)n(C2CCN(C)C(=O)C2)c(=O)[nH]1)C1CCCCC1] | general procedure B Example 2 | 350.2 |
| 24 | 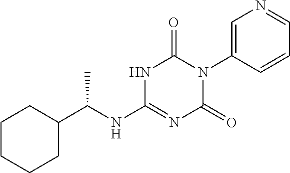 [SMILES: C[C@H](Nc1nc(=O)n(-c2cccnc2)c(=O)[nH]1)C1CCCCC1] | general procedure A Example 2 | 316.4 |
| 25 | 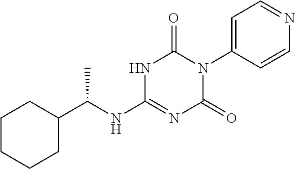 [SMILES: C[C@H](Nc1nc(=O)n(-c2ccncc2)c(=O)[nH]1)C1CCCCC1] | general procedure A Example 2 | 316.4 |
| 26 | 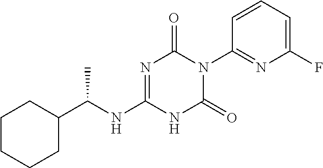 [SMILES: C[C@H](Nc1nc(=O)n(-c2cccc(F)n2)c(=O)[nH]1)C1CCCCC1] | general procedure A Example 2 | 334.2 |
| 27 | 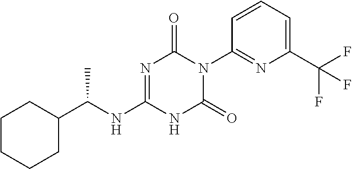 [SMILES: C[C@H](Nc1nc(=O)n(-c2cccc(C(F)(F)F)n2)c(=O)[nH]1)C1CCCCC1] | general procedure A Example 2 | 384.2 |
| 28 | 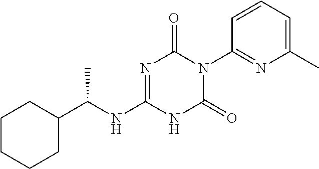 [SMILES: Cc1cccc(-n2c(=O)nc(N[C@@H](C)C3CCCCC3)[nH]c2=O)n1] | general procedure A Example 2 | 330.2 |
| 29 | 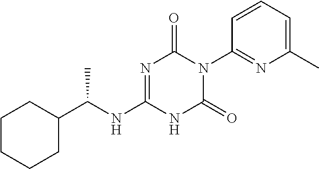 [SMILES: Cc1cccc(-n2c(=O)nc(N[C@@H](C)C3CCCCC3)[nH]c2=O)n1] | general procedure A Example 2 | 330.2 |
| 30 | 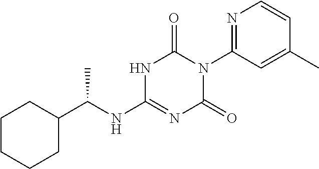 [SMILES: Cc1ccnc(-n2c(=O)nc(N[C@@H](C)C3CCCCC3)[nH]c2=O)c1] | general procedure A Example 2 | 330.4 |
| 31 | 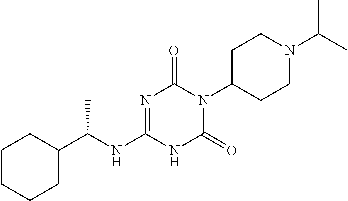 [SMILES: CC(C)N1CCC(n2c(=O)nc(N[C@@H](C)C3CCCCC3)[nH]c2=O)CC1] | general procedure B Example 2 | 364.5 |
| 32 | 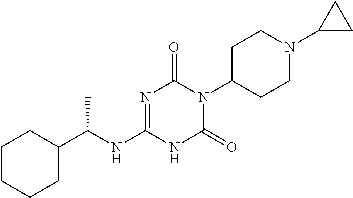 [SMILES: C[C@H](Nc1nc(=O)n(C2CCN(C3CC3)CC2)c(=O)[nH]1)C1CCCCC1] | general procedure B Example 2 | 362.5 |
| 33 | 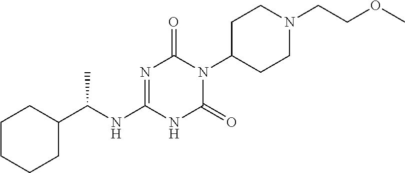 [SMILES: COCCN1CCC(n2c(=O)nc(N[C@@H](C)C3CCCCC3)[nH]c2=O)CC1] | general procedure B Example 2 | 380.5 |
| 34 | 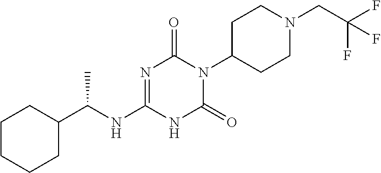 [SMILES: C[C@H](Nc1nc(=O)n(C2CCN(CC(F)(F)F)CC2)c(=O)[nH]1)C1CCCCC1] | general procedure B Example 2 | 404.5 |
| 35 | 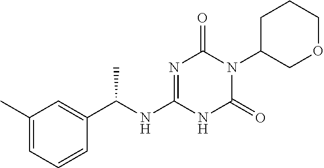 [SMILES: Cc1cccc([C@H](C)Nc2nc(=O)n(C3CCCOC3)c(=O)[nH]2)c1] | general procedure B Example 2 | 331.4 |
| 36 | 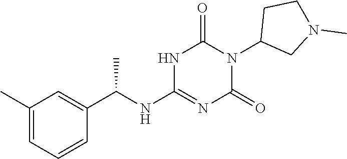 | general procedure B Example 2 | 330.2 |
| 37 | 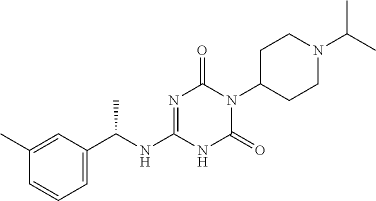 [SMILES: Cc1cccc([C@H](C)Nc2nc(=O)n(C3CCN(C(C)C)CC3)c(=O)[nH]2)c1] | general procedure B Example 2 | 372.5 |
| 38 | 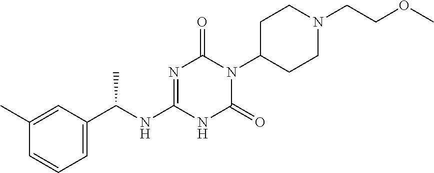 | general procedure B Example 2 | 388.5 |
| 39 | 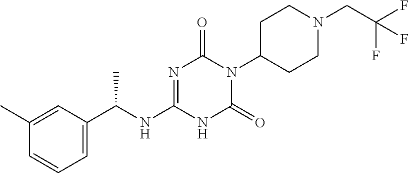 [SMILES: Cc1cccc([C@H](C)Nc2nc(=O)n(C3CCN(CC(F)(F)F)CC3)c(=O)[nH]2)c1] | general procedure B Example 2 | 412.4 |
| 40 | 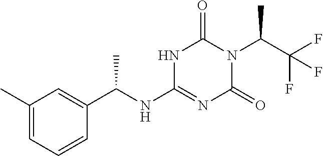 [SMILES: Cc1cccc([C@H](C)Nc2nc(=O)n([C@@H](C)C(F)(F)F)c(=O)[nH]2)c1] | general procedure B Example 2 | 343.1 |
| 41 | 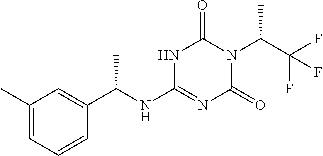 [SMILES: Cc1cccc([C@H](C)Nc2nc(=O)n([C@H](C)C(F)(F)F)c(=O)[nH]2)c1] | general procedure B Example 2 | 343.3 |
| 42 | 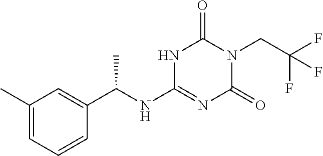 [SMILES: Cc1cccc([C@H](C)Nc2nc(=O)n(CC(F)(F)F)c(=O)[nH]2)c1] | general procedure B Example 2 | 329.3 |
| 43 | 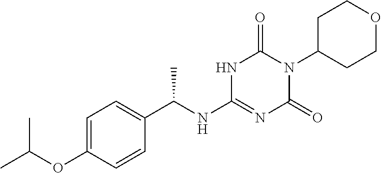 [SMILES: CC(C)Oc1ccc([C@H](C)Nc2nc(=O)n(C3CCOCC3)c(=O)[nH]2)cc1] | general procedure B Example 2 | 375.2 |
| 44 | 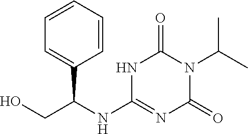 [SMILES: CC(C)n1c(=O)nc(N[C@@H](CO)c2ccccc2)[nH]c1=O] | general procedure B Example 2 | 291.3 |
| 45 | 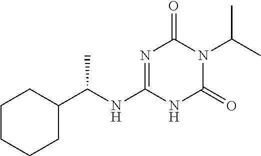 [SMILES: CC(C)n1c(=O)nc(N[C@@H](C)C2CCCCC2)[nH]c1=O] | general procedure B Example 2 | 281.4 |
| 46 | 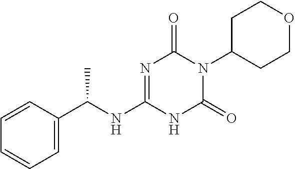 [SMILES: C[C@H](Nc1nc(=O)n(C2CCOCC2)c(=O)[nH]1)c1ccccc1] | general procedure B Example 2 | 317.4 |
| 47 | 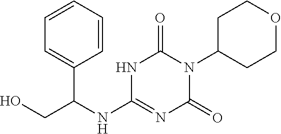 [SMILES: O=c1nc(NC(CO)c2ccccc2)[nH]c(=O)n1C1CCOCC1] | general procedure B Example 2 | 333.4 |
| 48 | 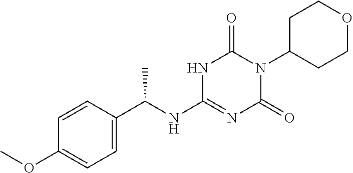 [SMILES: COc1ccc([C@H](C)Nc2nc(=O)n(C3CCOCC3)c(=O)[nH]2)cc1] | general procedure B Example 2 | 347.2 |
| 49 | 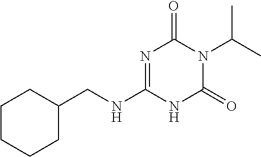 [SMILES: CC(C)n1c(=O)nc(NCC2CCCCC2)[nH]c1=O] | general procedure B Example 2 | 267.4 |
| 50 | 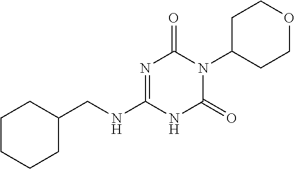 [SMILES: O=c1nc(NCC2CCCCC2)[nH]c(=O)n1C1CCOCC1] | general procedure B Example 2 | 309.4 |
| 51 | 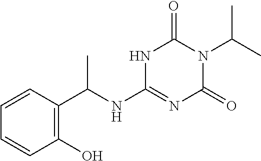 [SMILES: CC(Nc1nc(=O)n(C(C)C)c(=O)[nH]1)c1ccccc1O] | general procedure B Example 2 | 291.3 |
| 52 | 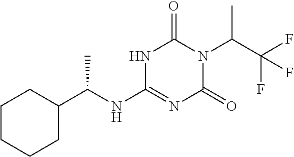 [SMILES: CC(n1c(=O)nc(N[C@@H](C)C2CCCCC2)[nH]c1=O)C(F)(F)F] | general procedure B Example 2 | 335.3 |
| 53 | 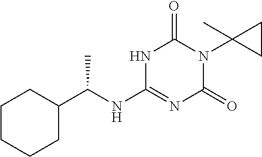 [SMILES: C[C@H](Nc1nc(=O)n(C2(C)CC2)c(=O)[nH]1)C1CCCCC1] | general procedure B Example 2 | 293.4 |
| 54 | 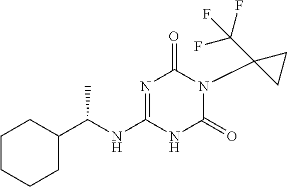 [SMILES: C[C@H](Nc1nc(=O)n(C2(C(F)(F)F)CC2)c(=O)[nH]1)C1CCCCC1] | general procedure B Example 2 | 347.4 |
| 55 | 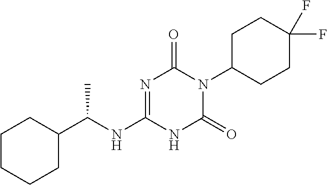 [SMILES: C[C@H](Nc1nc(=O)n(C2CCC(F)(F)CC2)c(=O)[nH]1)C1CCCCC1] | general procedure B Example 2 | 357.4 |
| 56 | 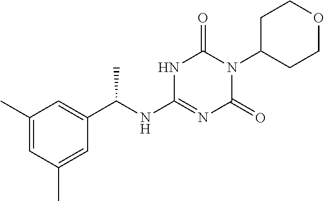 [SMILES: Cc1cc(C)cc([C@H](C)Nc2nc(=O)n(C3CCOCC3)c(=O)[nH]2)c1] | general procedure B Example 2 | 345.2 |
| 57 | 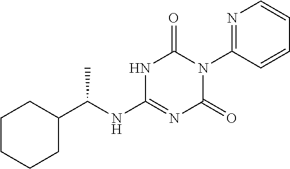 [SMILES: C[C@H](Nc1nc(=O)n(-c2ccccn2)c(=O)[nH]1)C1CCCCC1] | general procedure A Example 1 | 316.4 |
| 58 | 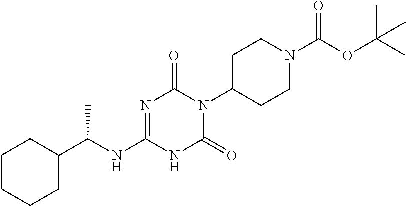 [SMILES: C[C@H](Nc1nc(=O)n(C2CCN(C(=O)OC(C)(C)C)CC2)c(=O)[nH]1)C1CCCCC1] | general procedure B Example 2 | 422.5 |
| 59 | 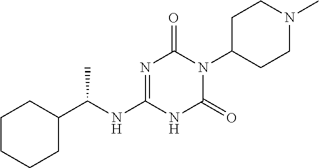 [SMILES: C[C@H](Nc1nc(=O)n(C2CCN(C)CC2)c(=O)[nH]1)C1CCCCC1] | general procedure B Example 2 | 336.5 |
| 60 | 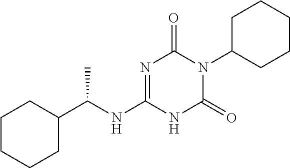 [SMILES: C[C@H](Nc1nc(=O)n(C2CCCCC2)c(=O)[nH]1)C1CCCCC1] | general procedure B Example 2 | 321.4 |
| 61 | 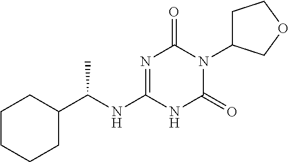 [SMILES: C[C@H](Nc1nc(=O)n(C2CCOC2)c(=O)[nH]1)C1CCCCC1] | general procedure B Example 2 | 309.4 |
| 62 | 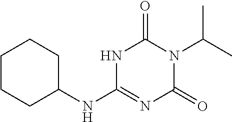 [SMILES: CC(C)n1c(=O)nc(NC2CCCCC2)[nH]c1=O] | general procedure B Example 2 | 253.3 |
| 63 | 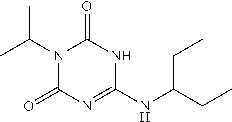 [SMILES: CCC(CC)Nc1nc(=O)n(C(C)C)c(=O)[nH]1] | general procedure B Example 2 | 241.3 |
| 64 | 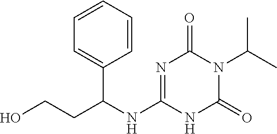 [SMILES: CC(C)n1c(=O)nc(NC(CCO)c2ccccc2)[nH]c1=O] | general procedure B Example 2 | 305.2 |
| 65 | 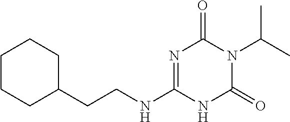 [SMILES: CC(C)n1c(=O)nc(NCCC2CCCCC2)[nH]c1=O] | general procedure B Example 2 | 281.4 |
| 66 | 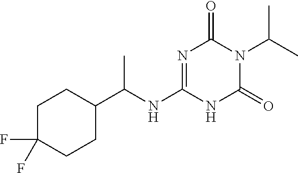 [SMILES: CC(Nc1nc(=O)n(C(C)C)c(=O)[nH]1)C1CCC(F)(F)CC1] | general procedure B Example 2 | 317.4 |
| 67 | 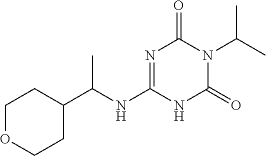 [SMILES: CC(Nc1nc(=O)n(C(C)C)c(=O)[nH]1)C1CCOCC1] | general procedure B Example 2 | 283.3 |
| 68 | 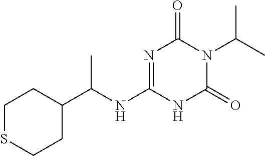 [SMILES: CC(Nc1nc(=O)n(C(C)C)c(=O)[nH]1)C1CCSCC1] | general procedure B Example 2 | 299.4 |
| 69 | 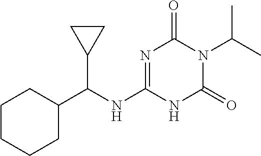 [SMILES: CC(C)n1c(=O)nc(NC(C2CCCCC2)C2CC2)[nH]c1=O] | general procedure B Example 2 | 307.4 |
| 70 | 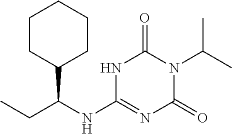 [SMILES: CC[C@H](Nc1nc(=O)n(C(C)C)c(=O)[nH]1)C1CCCCC1] | general procedure B Example 2 | 295.4 |
| 71 | 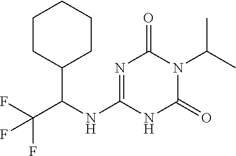 [SMILES: CC(C)n1c(=O)nc(NC(C2CCCCC2)C(F)(F)F)[nH]c1=O] | general procedure B Example 2 | 333.2 |
| 72 | 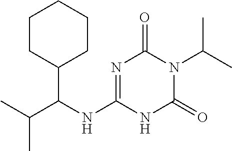 [SMILES: CC(C)C(Nc1nc(=O)n(C(C)C)c(=O)[nH]1)C1CCCCC1] | general procedure B Example 2 | 309.4 |
| 73 | 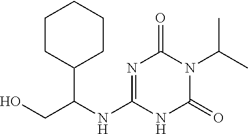 [SMILES: CC(C)n1c(=O)nc(NC(CO)C2CCCCC2)[nH]c1=O] | general procedure B Example 2 | 297.4 |
| 74 | 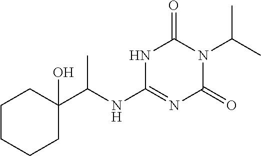 [SMILES: CC(C)n1c(=O)nc(NC(C)C2(O)CCCCC2)[nH]c1=O] | general procedure B Example 2 | 297.4 |
| 75 | 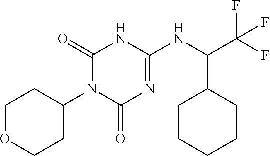 [SMILES: O=c1nc(NC(C2CCCCC2)C(F)(F)F)[nH]c(=O)n1C1CCOCC1] | general procedure B Example 2 | 377.4 |
| 76 | 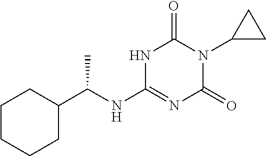 [SMILES: C[C@H](Nc1nc(=O)n(C2CC2)c(=O)[nH]1)C1CCCCC1] | general procedure B Example 2 | 279.2 |
| 77 | 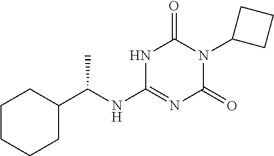 [SMILES: C[C@H](Nc1nc(=O)n(C2CCC2)c(=O)[nH]1)C1CCCCC1] | general procedure B Example 2 | 293.2 |
| 78 | 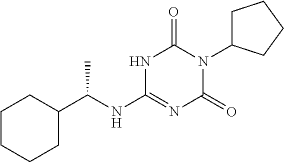 [SMILES: C[C@H](Nc1nc(=O)n(C2CCCC2)c(=O)[nH]1)C1CCCCC1] | general procedure B Example 2 | 307.4 |
| 79 | 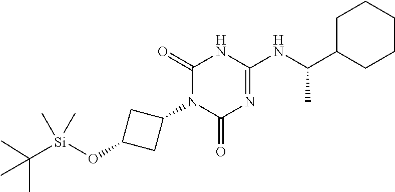 [SMILES: C[C@H](Nc1nc(=O)n([C@H]2C[C@@H](O[Si](C)(C)C(C)(C)C)C2)c(=O)[nH]1)C1CCCCC1] | general procedure B Example 2 | 423.7 |
| 80 | 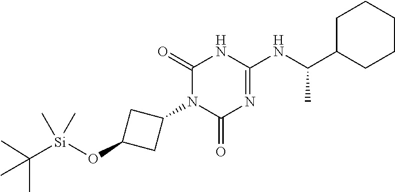 [SMILES: C[C@H](Nc1nc(=O)n([C@H]2C[C@H](O[Si](C)(C)C(C)(C)C)C2)c(=O)[nH]1)C1CCCCC1] | general procedure B Example 2 | 423.7 |
| 81 | 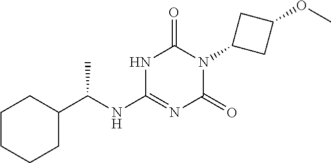 [SMILES: CO[C@H]1C[C@@H](n2c(=O)nc(N[C@@H](C)C3CCCCC3)[nH]c2=O)C1] | general procedure B Example 2 | 323.4 |
| 82 | 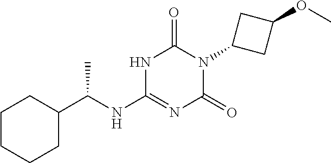 [SMILES: CO[C@H]1C[C@H](n2c(=O)nc(N[C@@H](C)C3CCCCC3)[nH]c2=O)C1] | general procedure B Example 2 | 323.4 |
| 83 | 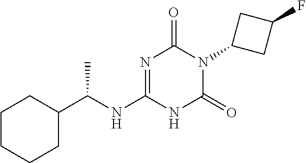 [SMILES: C[C@H](Nc1nc(=O)n([C@H]2C[C@H](F)C2)c(=O)[nH]1)C1CCCCC1] | general procedure B Example 2 | 311.4 |
| 84 | 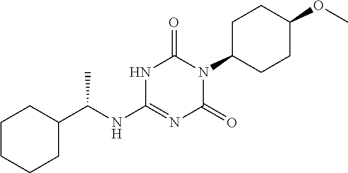 [SMILES: CO[C@H]1CC[C@@H](n2c(=O)nc(N[C@@H](C)C3CCCCC3)[nH]c2=O)CC1] | general procedure B Example 2 | 351.5 |
| 85 | 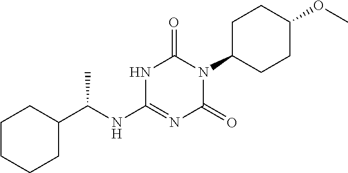 [SMILES: CO[C@H]1CC[C@H](n2c(=O)nc(N[C@@H](C)C3CCCCC3)[nH]c2=O)CC1] | general procedure B Example 2 | 351.5 |
| 86 | 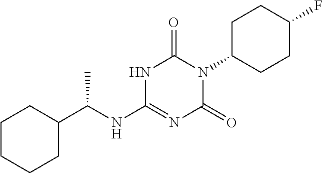 [SMILES: C[C@H](Nc1nc(=O)n([C@H]2CC[C@@H](F)CC2)c(=O)[nH]1)C1CCCCC1] | general procedure B Example 2 | 339.4 |
| 87 | 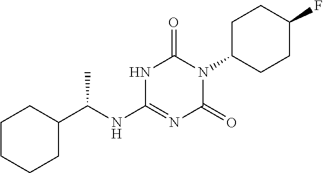 [SMILES: C[C@H](Nc1nc(=O)n([C@H]2CC[C@H](F)CC2)c(=O)[nH]1)C1CCCCC1] | general procedure B Example 2 | 339.4 |
| 88 | 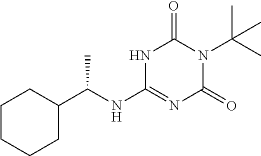 [SMILES: C[C@H](Nc1nc(=O)n(C(C)(C)C)c(=O)[nH]1)C1CCCCC1] | general procedure B Example 2 | 295.4 |
| 89 | 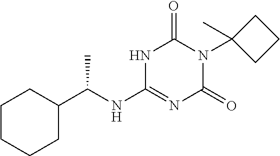 [SMILES: C[C@H](Nc1nc(=O)n(C2(C)CCC2)c(=O)[nH]1)C1CCCCC1] | general procedure B Example 2 | 307.4 |
| 90 | 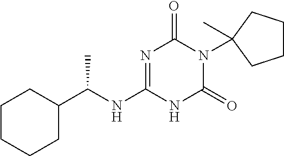 [SMILES: C[C@H](Nc1nc(=O)n(C2(C)CCCC2)c(=O)[nH]1)C1CCCCC1] | general procedure B Example 2 | 321.4 |
| 91 | 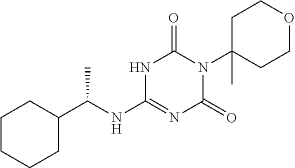 [SMILES: C[C@H](Nc1nc(=O)n(C2(C)CCOCC2)c(=O)[nH]1)C1CCCCC1] | general procedure B Example 2 | 337.4 |
| 92 | 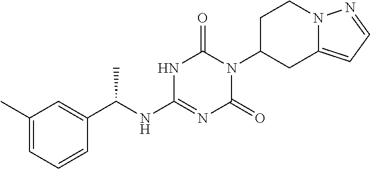 [SMILES: Cc1cccc([C@H](C)Nc2nc(=O)n(C3CCn4nccc4C3)c(=O)[nH]2)c1] | general procedure B Example 2 | 367.2 |
| 93 | 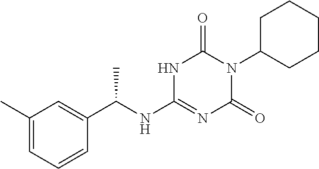 [SMILES: Cc1cccc([C@H](C)Nc2nc(=O)n(C3CCCCC3)c(=O)[nH]2)c1] | general procedure B Example 2 | 329.2 |
| 94 | 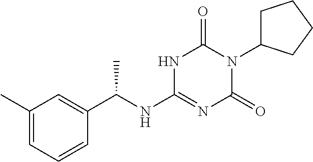 [SMILES: Cc1cccc([C@H](C)Nc2nc(=O)n(C3CCCC3)c(=O)[nH]2)c1] | general procedure B Example 2 | 315.4 |
| 95 | 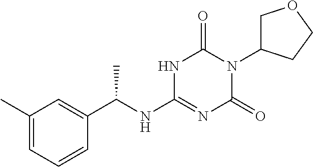 [SMILES: Cc1cccc([C@H](C)Nc2nc(=O)n(C3CCOC3)c(=O)[nH]2)c1] | general procedure B Example 2 | 317.2 |
| 96 | 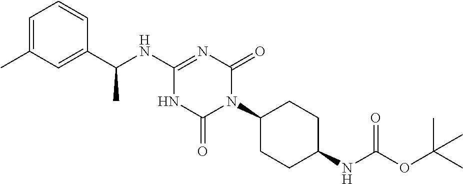 [SMILES: Cc1cccc([C@H](C)Nc2nc(=O)n([C@H]3CC[C@@H](NC(=O)OC(C)(C)C)CC3)c(=O)[nH]2)c1] | general procedure B Example 2 | 444.6 |
| 97 | 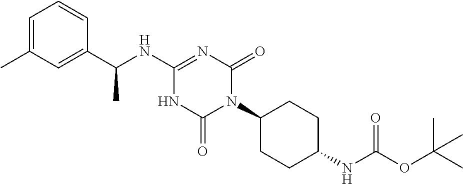 [SMILES: Cc1cccc([C@H](C)Nc2nc(=O)n([C@H]3CC[C@H](NC(=O)OC(C)(C)C)CC3)c(=O)[nH]2)c1] | general procedure B Example 2 | 444.6 |
| 98 | 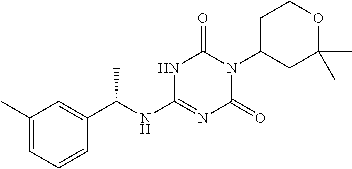 | general procedure B Example 2 | 359.2 |
| 99 | 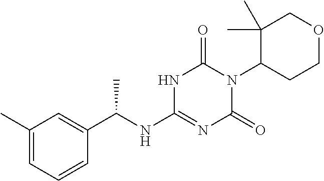 [SMILES: Cc1cccc([C@H](C)Nc2nc(=O)n(C3CCOCC3(C)C)c(=O)[nH]2)c1] | general procedure B Example 2 | 359.2 |
| 100 | 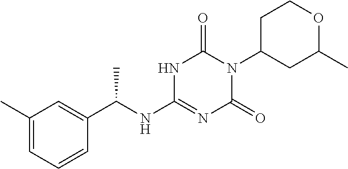 | general procedure B Example 2 | 345.2 |
| 101 | 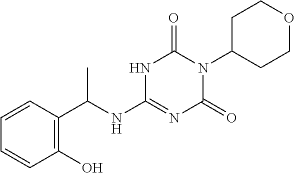 [SMILES: CC(Nc1nc(=O)n(C2CCOCC2)c(=O)[nH]1)c1ccccc1O] | general procedure B Example 2 | 333.4 |
| 102 | 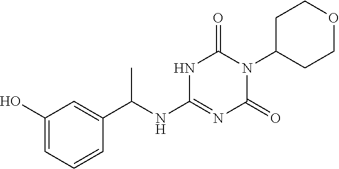 [SMILES: CC(Nc1nc(=O)n(C2CCOCC2)c(=O)[nH]1)c1cccc(O)c1] | general procedure B Example 2 | 333.4 |
| 103 | 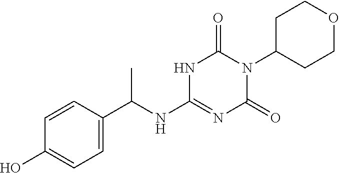 [SMILES: CC(Nc1nc(=O)n(C2CCOCC2)c(=O)[nH]1)c1ccc(O)cc1] | general procedure B Example 2 | 333.4 |
| 104 | 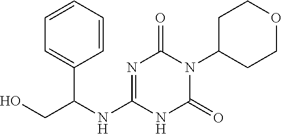 [SMILES: O=c1nc(NC(CO)c2ccccc2)[nH]c(=O)n1C1CCOCC1] | general procedure B Example 2 | 333.2 |
| 105 | 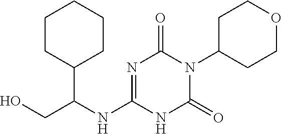 [SMILES: O=c1nc(NC(CO)C2CCCCC2)[nH]c(=O)n1C1CCOCC1] | general procedure B Example 2 | 339.4 |
| 106 | 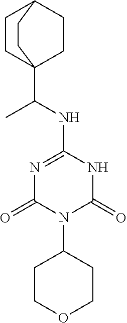 [SMILES: CC(Nc1nc(=O)n(C2CCOCC2)c(=O)[nH]1)C12CCC(CC1)CC2] | general procedure B Example 2 | 349.5 |
| 107 | 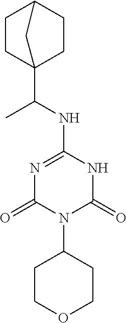 [SMILES: CC(Nc1nc(=O)n(C2CCOCC2)c(=O)[nH]1)C12CCC(CC1)C2] | general procedure B Example 2 | 335.4 |
| 108 | 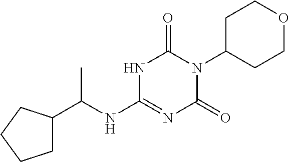 [SMILES: CC(Nc1nc(=O)n(C2CCOCC2)c(=O)[nH]1)C1CCCC1] | general procedure B Example 2 | 309.4 |
| 109 | 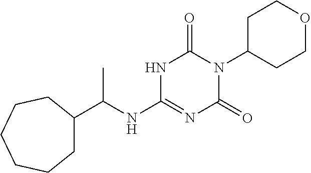 [SMILES: CC(Nc1nc(=O)n(C2CCOCC2)c(=O)[nH]1)C1CCCCCC1] | general procedure B Example 2 | 337.4 |
| 110 | 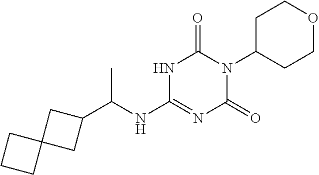 [SMILES: CC(Nc1nc(=O)n(C2CCOCC2)c(=O)[nH]1)C1CC2(CCC2)C1] | general procedure B Example 2 | 335.4 |
| 111 | 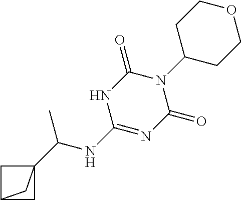 [SMILES: CC(Nc1nc(=O)n(C2CCOCC2)c(=O)[nH]1)C12CC(C1)C2] | general procedure B Example 2 | 307.4 |
| 112 | 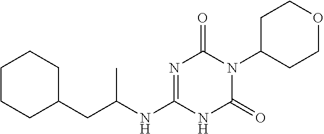 [SMILES: CC(CC1CCCCC1)Nc1nc(=O)n(C2CCOCC2)c(=O)[nH]1] | general procedure B Example 2 | 337.4 |
| 113 | 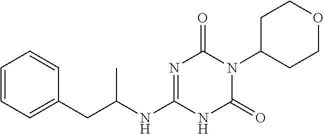 [SMILES: CC(Cc1ccccc1)Nc1nc(=O)n(C2CCOCC2)c(=O)[nH]1] | general procedure B Example 2 | 331.4 |
| 114 | 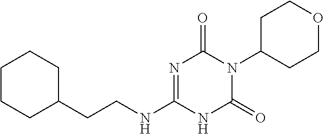 [SMILES: O=c1nc(NCCC2CCCCC2)[nH]c(=O)n1C1CCOCC1] | general procedure B Example 2 | 323.4 |
| 115 | 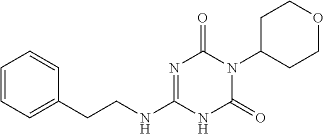 [SMILES: O=c1nc(NCCc2ccccc2)[nH]c(=O)n1C1CCOCC1] | general procedure B Example 2 | 317.4 |
| 116 | 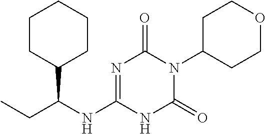 [SMILES: CC[C@H](Nc1nc(=O)n(C2CCOCC2)c(=O)[nH]1)C1CCCCC1] | general procedure B Example 2 | 337.2 |
| 117 | 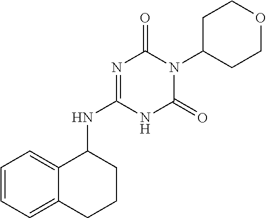 [SMILES: O=c1nc(NC2CCCc3ccccc32)[nH]c(=O)n1C1CCOCC1] | general procedure B Example 2 | 343.2 |
| 118 | 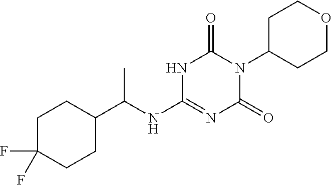 [SMILES: CC(Nc1nc(=O)n(C2CCOCC2)c(=O)[nH]1)C1CCC(F)(F)CC1] | general procedure B Example 2 | 359.4 |
| 119 | 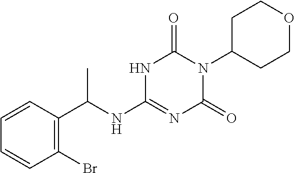 [SMILES: CC(Nc1nc(=O)n(C2CCOCC2)c(=O)[nH]1)c1ccccc1Br] | general procedure B Example 2 | 395.1 |
| 120 | 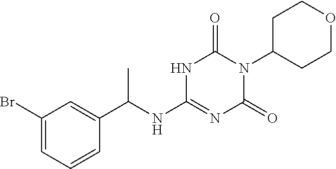 [SMILES: CC(Nc1nc(=O)n(C2CCOCC2)c(=O)[nH]1)c1cccc(Br)c1] | general procedure B Example 2 | 395.3 |
| 121 | 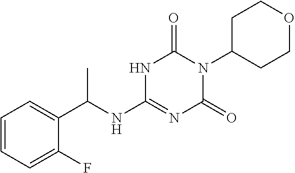 [SMILES: CC(Nc1nc(=O)n(C2CCOCC2)c(=O)[nH]1)c1ccccc1F] | general procedure B Example 2 | 335.4 |
| 122 | 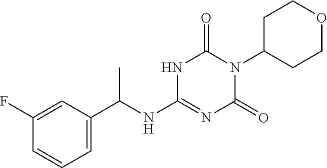 [SMILES: CC(Nc1nc(=O)n(C2CCOCC2)c(=O)[nH]1)c1cccc(F)c1] | general procedure B Example 2 | 335.1 |
| 123 | 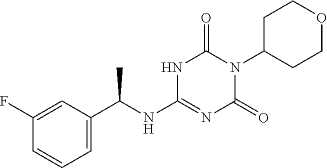 [SMILES: C[C@@H](Nc1nc(=O)n(C2CCOCC2)c(=O)[nH]1)c1cccc(F)c1] | general procedure B Example 2 | 335.4 |
| 124 | 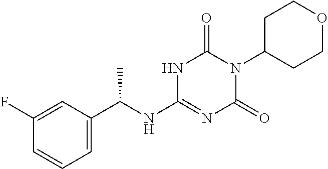 [SMILES: C[C@H](Nc1nc(=O)n(C2CCOCC2)c(=O)[nH]1)c1cccc(F)c1] | general procedure B Example 2 | 335.4 |
| 125 | 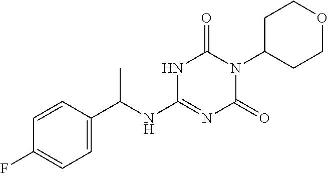 [SMILES: CC(Nc1nc(=O)n(C2CCOCC2)c(=O)[nH]1)c1ccc(F)cc1] | general procedure B Example 2 | 335.1 |
| 126 | 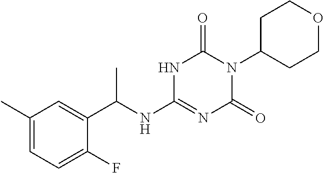 [SMILES: Cc1ccc(F)c(C(C)Nc2nc(=O)n(C3CCOCC3)c(=O)[nH]2)c1] | general procedure B Example 2 | 349.4 |
| 127 | 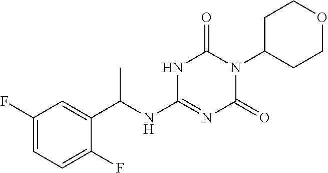 [SMILES: CC(Nc1nc(=O)n(C2CCOCC2)c(=O)[nH]1)c1cc(F)ccc1F] | general procedure B Example 2 | 353.3 |
| 128 | 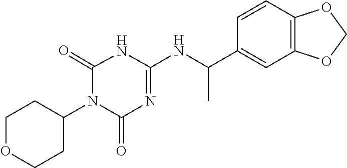 [SMILES: CC(Nc1nc(=O)n(C2CCOCC2)c(=O)[nH]1)c1ccc2c(c1)OCO2] | general procedure B Example 2 | 361.4 |
| 129 | 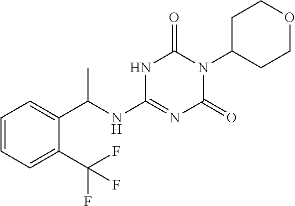 [SMILES: CC(Nc1nc(=O)n(C2CCOCC2)c(=O)[nH]1)c1ccccc1C(F)(F)F] | general procedure B Example 2 | 385.4 |
| 130 | 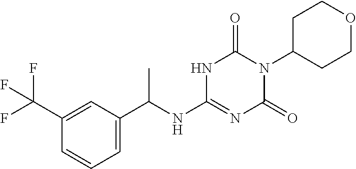 [SMILES: CC(Nc1nc(=O)n(C2CCOCC2)c(=O)[nH]1)c1cccc(C(F)(F)F)c1] | general procedure B Example 2 | 385.4 |
| 131 | 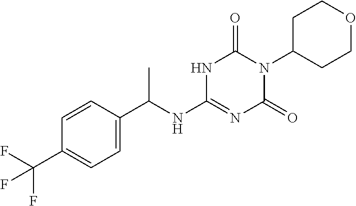 [SMILES: CC(Nc1nc(=O)n(C2CCOCC2)c(=O)[nH]1)c1ccc(C(F)(F)F)cc1] | general procedure B Example 2 | 385.1 |
| 132 | 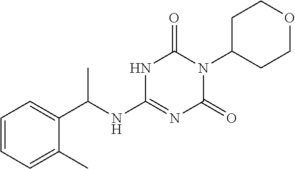 [SMILES: Cc1ccccc1C(C)Nc1nc(=O)n(C2CCOCC2)c(=O)[nH]1] | general procedure B Example 2 | 331.4 |
| 133 | 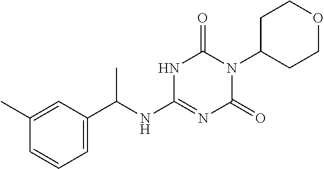 [SMILES: Cc1cccc(C(C)Nc2nc(=O)n(C3CCOCC3)c(=O)[nH]2)c1] | general procedure B Example 2 | 331.4 |
| 134 | 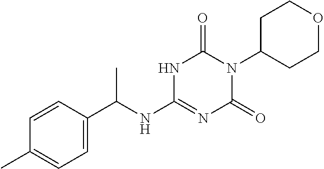 [SMILES: Cc1ccc(C(C)Nc2nc(=O)n(C3CCOCC3)c(=O)[nH]2)cc1] | general procedure B Example 2 | 331.4 |
| 135 | 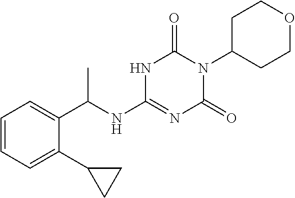 [SMILES: CC(Nc1nc(=O)n(C2CCOCC2)c(=O)[nH]1)c1ccccc1C1CC1] | general procedure B Example 2 | 357.4 |
| 136 | 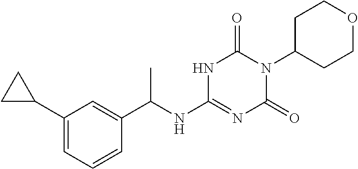 [SMILES: CC(Nc1nc(=O)n(C2CCOCC2)c(=O)[nH]1)c1cccc(C2CC2)c1] | general procedure B Example 2 | 357.4 |
| 137 | 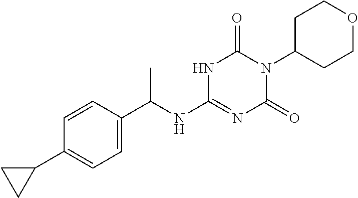 [SMILES: CC(Nc1nc(=O)n(C2CCOCC2)c(=O)[nH]1)c1ccc(C2CC2)cc1] | general procedure B Example 2 | 357.4 |
| 138 | 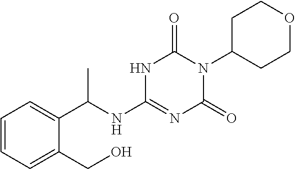 [SMILES: CC(Nc1nc(=O)n(C2CCOCC2)c(=O)[nH]1)c1ccccc1CO] | general procedure B Example 2 | 347.2 |
| 139 | 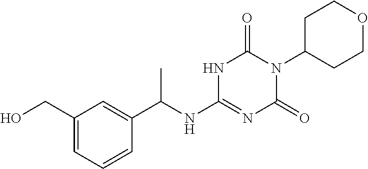 [SMILES: CC(Nc1nc(=O)n(C2CCOCC2)c(=O)[nH]1)c1cccc(CO)c1] | general procedure B Example 2 | 347.4 |
| 140 | 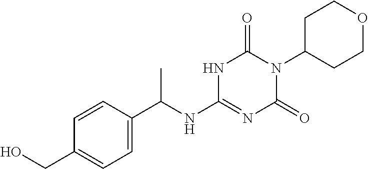 [SMILES: CC(Nc1nc(=O)n(C2CCOCC2)c(=O)[nH]1)c1ccc(CO)cc1] | general procedure B Example 2 | 347.4 |
| 141 | 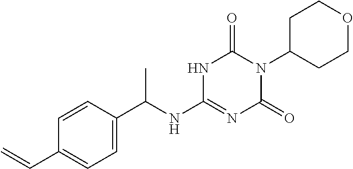 [SMILES: C=Cc1ccc(C(C)Nc2nc(=O)n(C3CCOCC3)c(=O)[nH]2)cc1] | general procedure B Example 2 | 343.4 |
| 142 | 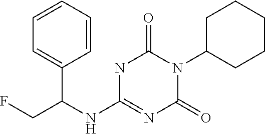 [SMILES: O=c1nc(NC(CF)c2ccccc2)[nH]c(=O)n1C1CCCCC1] | general procedure B Example 2 | 333.4 |
| 143 | 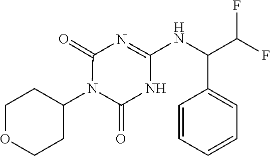 [SMILES: O=c1nc(NC(c2ccccc2)C(F)F)[nH]c(=O)n1C1CCOCC1] | general procedure B Example 2 | 353.3 |
| 144 | 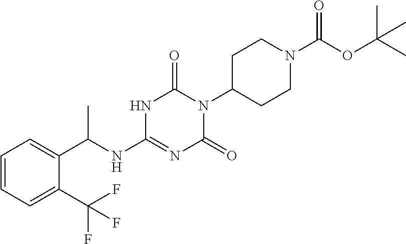 [SMILES: CC(Nc1nc(=O)n(C2CCN(C(=O)OC(C)(C)C)CC2)c(=O)[nH]1)c1ccccc1C(F)(F)F] | general procedure B Example 2 | 484.2 |
| 145 | 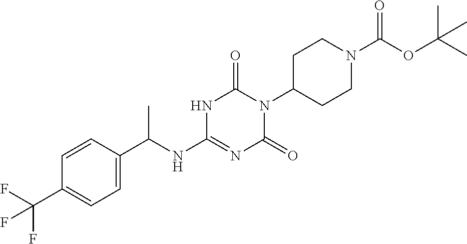 [SMILES: CC(Nc1nc(=O)n(C2CCN(C(=O)OC(C)(C)C)CC2)c(=O)[nH]1)c1ccc(C(F)(F)F)cc1] | general procedure B Example 2 | 484.5 |
| 146 | 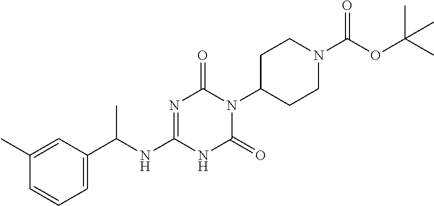 [SMILES: Cc1cccc(C(C)Nc2nc(=O)n(C3CCN(C(=O)OC(C)(C)C)CC3)c(=O)[nH]2)c1] | general procedure B Example 2 | 430.5 |
| 147 | 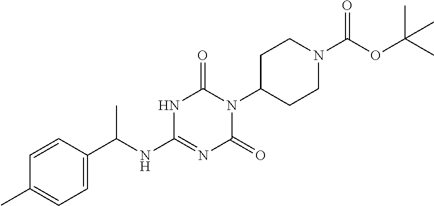 [SMILES: Cc1ccc(C(C)Nc2nc(=O)n(C3CCN(C(=O)OC(C)(C)C)CC3)c(=O)[nH]2)cc1] | general procedure B Example 2 | 430.2 |
| 148 | 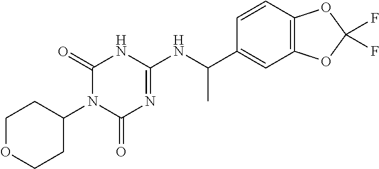 [SMILES: CC(Nc1nc(=O)n(C2CCOCC2)c(=O)[nH]1)c1ccc2c(c1)OC(F)(F)O2] | general procedure B Example 2 | 397.4 |
| 149 | 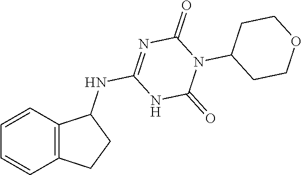 [SMILES: O=c1nc(NC2CCc3ccccc32)[nH]c(=O)n1C1CCOCC1] | general procedure B Example 2 | 329.4 |
| 150 | 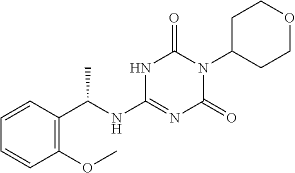 [SMILES: COc1ccccc1[C@H](C)Nc1nc(=O)n(C2CCOCC2)c(=O)[nH]1] | general procedure B Example 2 | 347.4 |
| 151 | 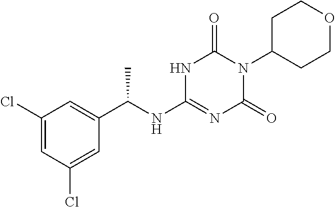 [SMILES: C[C@H](Nc1nc(=O)n(C2CCOCC2)c(=O)[nH]1)c1cc(Cl)cc(Cl)c1] | general procedure B Example 2 | 385.1 |
| 152 | 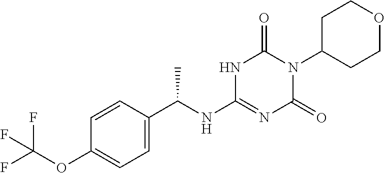 [SMILES: C[C@H](Nc1nc(=O)n(C2CCOCC2)c(=O)[nH]1)c1ccc(OC(F)(F)F)cc1] | general procedure B Example 2 | 401.4 |
| 153 | 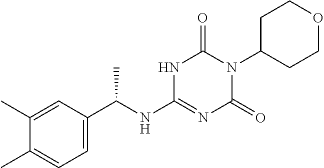 [SMILES: Cc1ccc([C@H](C)Nc2nc(=O)n(C3CCOCC3)c(=O)[nH]2)cc1C] | general procedure B Example 2 | 345.2 |
| 154 | 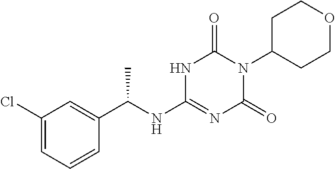 [SMILES: C[C@H](Nc1nc(=O)n(C2CCOCC2)c(=O)[nH]1)c1cccc(Cl)c1] | general procedure B Example 2 | 351.1 |
| 155 | 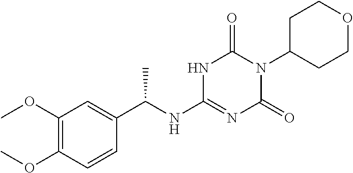 [SMILES: COc1ccc([C@H](C)Nc2nc(=O)n(C3CCOCC3)c(=O)[nH]2)cc1OC] | general procedure B Example 2 | 377.4 |
| 156 | 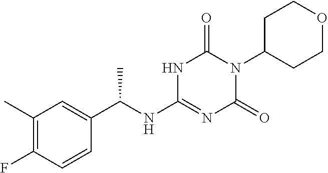 [SMILES: Cc1cc([C@H](C)Nc2nc(=O)n(C3CCOCC3)c(=O)[nH]2)ccc1F] | general procedure B Example 2 | 349.4 |
| 157 | 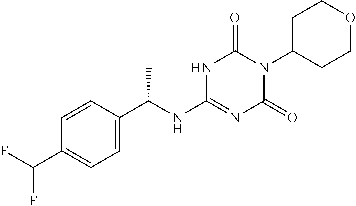 [SMILES: C[C@H](Nc1nc(=O)n(C2CCOCC2)c(=O)[nH]1)c1ccc(C(F)F)cc1] | general procedure B Example 2 | 367.2 |
| 158 | 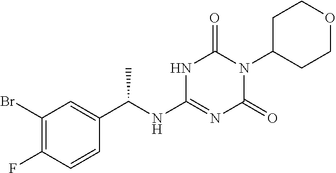 [SMILES: C[C@H](Nc1nc(=O)n(C2CCOCC2)c(=O)[nH]1)c1ccc(F)c(Br)c1] | general procedure B Example 2 | 413.1 |
| 159 | 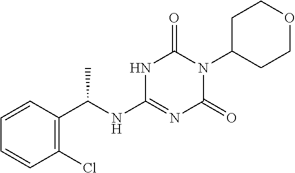 [SMILES: C[C@H](Nc1nc(=O)n(C2CCOCC2)c(=O)[nH]1)c1ccccc1Cl] | general procedure B Example 2 | 351.1 |
| 160 | 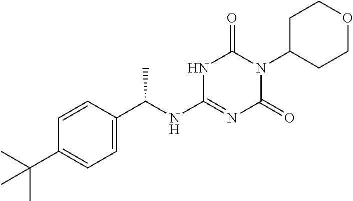 [SMILES: C[C@H](Nc1nc(=O)n(C2CCOCC2)c(=O)[nH]1)c1ccc(C(C)(C)C)cc1] | general procedure B Example 2 | 373.2 |
| 161 | 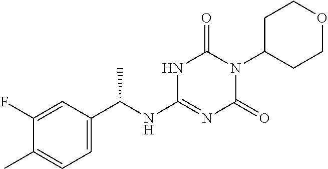 [SMILES: Cc1ccc([C@H](C)Nc2nc(=O)n(C3CCOCC3)c(=O)[nH]2)cc1F] | general procedure B Example 2 | 349.4 |
| 162 | 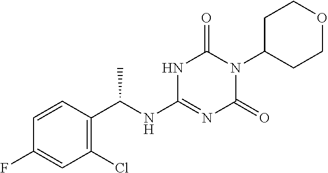 [SMILES: C[C@H](Nc1nc(=O)n(C2CCOCC2)c(=O)[nH]1)c1ccc(F)cc1Cl] | general procedure B Example 2 | 369.8 |
| 163 | 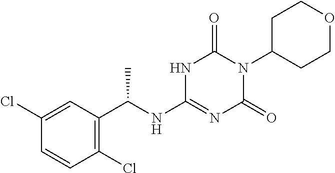 [SMILES: C[C@H](Nc1nc(=O)n(C2CCOCC2)c(=O)[nH]1)c1cc(Cl)ccc1Cl] | general procedure B Example 2 | 386.3 |
| 164 | 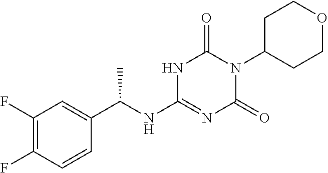 [SMILES: C[C@H](Nc1nc(=O)n(C2CCOCC2)c(=O)[nH]1)c1ccc(F)c(F)c1] | general procedure B Example 2 | 353.1 |
| 165 | 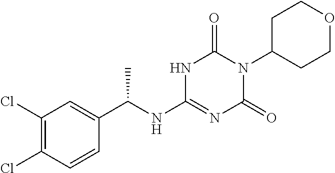 [SMILES: C[C@H](Nc1nc(=O)n(C2CCOCC2)c(=O)[nH]1)c1ccc(Cl)c(Cl)c1] | general procedure B Example 2 | 385.1 |
| 166 | 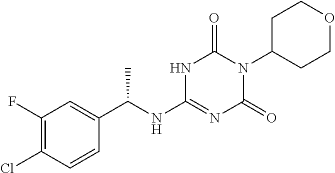 [SMILES: C[C@H](Nc1nc(=O)n(C2CCOCC2)c(=O)[nH]1)c1ccc(Cl)c(F)c1] | general procedure B Example 2 | 369.1 |
| 167 | 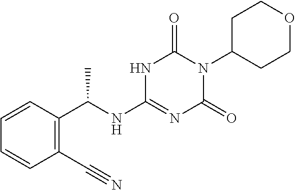 [SMILES: C[C@H](Nc1nc(=O)n(C2CCOCC2)c(=O)[nH]1)c1ccccc1C#N] | general procedure B Example 2 | 342.2 |
| 168 | 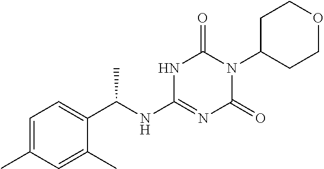 [SMILES: Cc1ccc([C@H](C)Nc2nc(=O)n(C3CCOCC3)c(=O)[nH]2)c(C)c1] | general procedure B Example 2 | 345.2 |
| 169 | 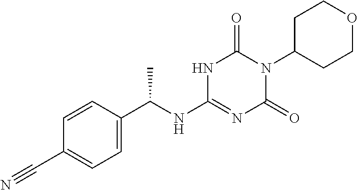 [SMILES: C[C@H](Nc1nc(=O)n(C2CCOCC2)c(=O)[nH]1)c1ccc(C#N)cc1] | general procedure B Example 2 | 342.2 |
| 170 | 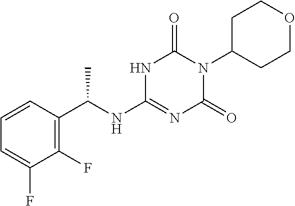 [SMILES: C[C@H](Nc1nc(=O)n(C2CCOCC2)c(=O)[nH]1)c1cccc(F)c1F] | general procedure B Example 2 | 353.1 |
| 171 | 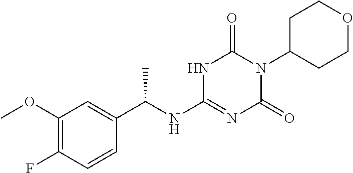 [SMILES: COc1cc([C@H](C)Nc2nc(=O)n(C3CCOCC3)c(=O)[nH]2)ccc1F] | general procedure B Example 2 | 365.2 |
| 172 | 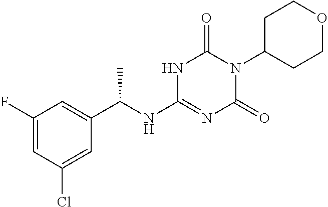 [SMILES: C[C@H](Nc1nc(=O)n(C2CCOCC2)c(=O)[nH]1)c1cc(F)cc(Cl)c1] | general procedure B Example 2 | 369.1 |
| 173 | 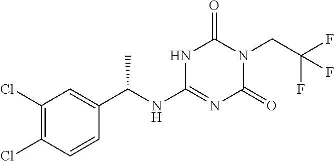 [SMILES: C[C@H](Nc1nc(=O)n(CC(F)(F)F)c(=O)[nH]1)c1ccc(Cl)c(Cl)c1] | general procedure B Example 2 | 384.2 |
| 174 | 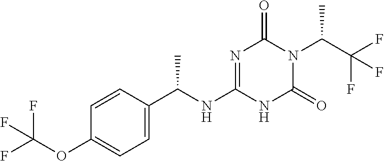 [SMILES: C[C@H](Nc1nc(=O)n([C@H](C)C(F)(F)F)c(=O)[nH]1)c1ccc(OC(F)(F)F)cc1] | general procedure B Example 2 | 413.1 |
| 175 | 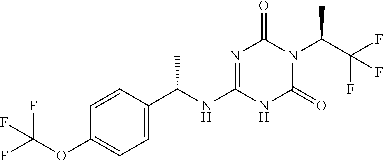 [SMILES: C[C@H](Nc1nc(=O)n([C@@H](C)C(F)(F)F)c(=O)[nH]1)c1ccc(OC(F)(F)F)cc1] | general procedure B Example 2 | 413.1 |
| 176 | 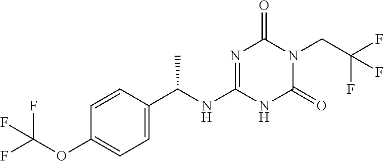 [SMILES: C[C@H](Nc1nc(=O)n(CC(F)(F)F)c(=O)[nH]1)c1ccc(OC(F)(F)F)cc1] | general procedure B Example 2 | 399.1 |
| 177 | 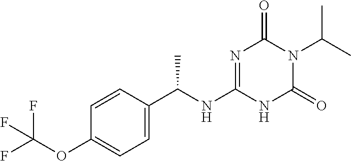 [SMILES: CC(C)n1c(=O)nc(N[C@@H](C)c2ccc(OC(F)(F)F)cc2)[nH]c1=O] | general procedure B Example 2 | 359.1 |
| 178 | 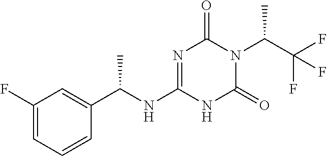 [SMILES: C[C@H](Nc1nc(=O)n([C@H](C)C(F)(F)F)c(=O)[nH]1)c1cccc(F)c1] | general procedure B Example 2 | 347.1 |
| 179 | 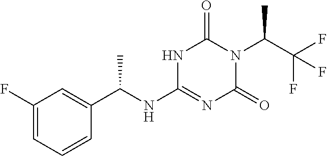 [SMILES: C[C@H](Nc1nc(=O)n([C@@H](C)C(F)(F)F)c(=O)[nH]1)c1cccc(F)c1] | general procedure B Example 2 | 347.1 |
| 180 | 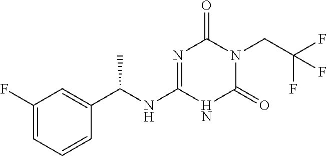 [SMILES: C[C@H](Nc1nc(=O)n(CC(F)(F)F)c(=O)[nH]1)c1cccc(F)c1] | general procedure B Example 2 | 333.1 |
| 181 | 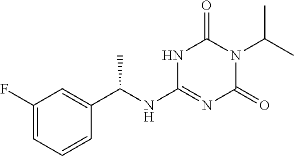 [SMILES: CC(C)n1c(=O)nc(N[C@@H](C)c2cccc(F)c2)[nH]c1=O] | general procedure B Example 2 | 293.1 |
| 182 | 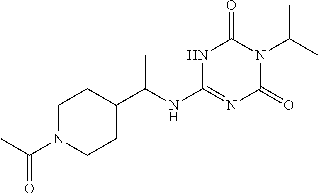 [SMILES: CC(=O)N1CCC(C(C)Nc2nc(=O)n(C(C)C)c(=O)[nH]2)CC1] | general procedure B | 324.4 |
| 183 | 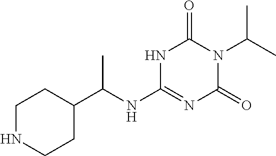 [SMILES: CC(Nc1nc(=O)n(C(C)C)c(=O)[nH]1)C1CCNCC1] | general procedure B, C | 282.4 |
| 184 | 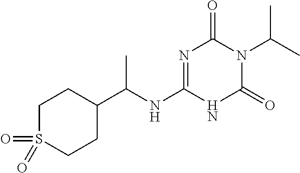 [SMILES: CC(Nc1nc(=O)n(C(C)C)c(=O)[nH]1)C1CCS(=O)(=O)CC1] | general procedure B | 331.4 |
| 185 | 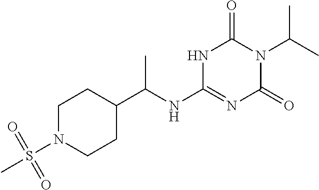 [SMILES: CC(Nc1nc(=O)n(C(C)C)c(=O)[nH]1)C1CCN(S(C)(=O)=O)CC1] | general procedure B, C | 360.5 |
| 186 | 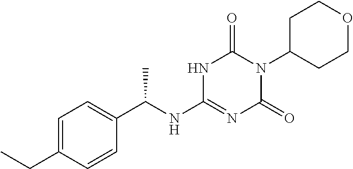 [SMILES: CCc1ccc([C@H](C)Nc2nc(=O)n(C3CCOCC3)c(=O)[nH]2)cc1] | general procedure B Example 2 | 345.2 |
| 187 | 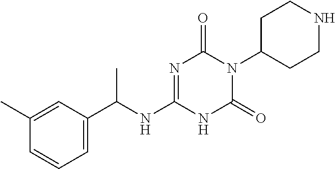 [SMILES: Cc1cccc(C(C)Nc2nc(=O)n(C3CCNCC3)c(=O)[nH]2)c1] | general procedure B, C | 330.2 |
| 188 | 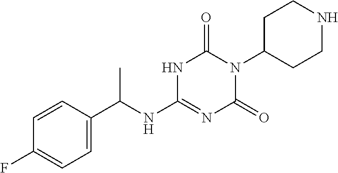 [SMILES: CC(Nc1nc(=O)n(C2CCNCC2)c(=O)[nH]1)c1ccc(F)cc1] | general procedure B, C | 334.4 |
| 189 | 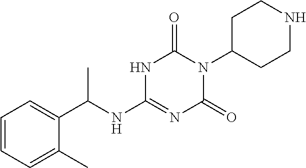 [SMILES: Cc1ccccc1C(C)Nc1nc(=O)n(C2CCNCC2)c(=O)[nH]1] | general procedure B, C | 330.4 |
| 190 | 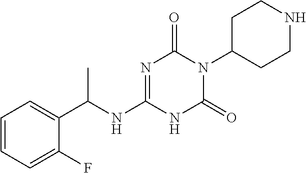 [SMILES: CC(Nc1nc(=O)n(C2CCNCC2)c(=O)[nH]1)c1ccccc1F] | general procedure B, C | 334.4 |
| 191 | 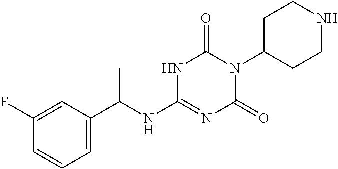 [SMILES: CC(Nc1nc(=O)n(C2CCNCC2)c(=O)[nH]1)c1cccc(F)c1] | general procedure B, C | 334.4 |
| 192 | 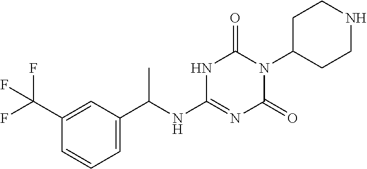 [SMILES: CC(Nc1nc(=O)n(C2CCNCC2)c(=O)[nH]1)c1cccc(C(F)(F)F)c1] | general procedure B, C | 384.4 |
| 193 | 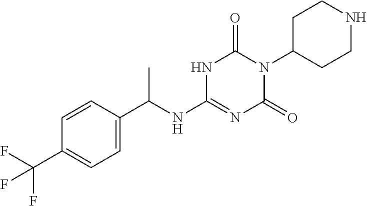 [SMILES: CC(Nc1nc(=O)n(C2CCNCC2)c(=O)[nH]1)c1ccc(C(F)(F)F)cc1] | general procedure B, C | 384.2 |
| 194 | 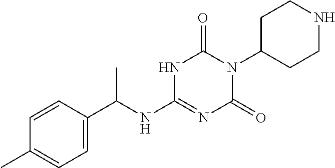 [SMILES: Cc1ccc(C(C)Nc2nc(=O)n(C3CCNCC3)c(=O)[nH]2)cc1] | general procedure B, C | 330.2 |
| 195 | 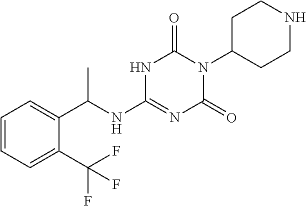 [SMILES: CC(Nc1nc(=O)n(C2CCNCC2)c(=O)[nH]1)c1ccccc1C(F)(F)F] | general procedure B, C | 384.4 |
| 196 | 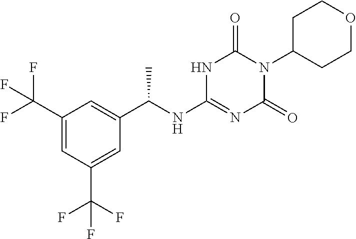 [SMILES: C[C@H](Nc1nc(=O)n(C2CCOCC2)c(=O)[nH]1)c1cc(C(F)(F)F)cc(C(F)(F)F)c1] | general procedure B Example 2 | 453.4 |
| 197 | 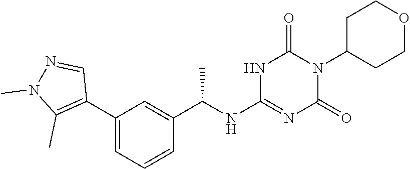 [SMILES: Cc1c(-c2cccc([C@H](C)Nc3nc(=O)n(C4CCOCC4)c(=O)[nH]3)c2)cnn1C] | general procedure B, E | 411.2 |
| 198 | 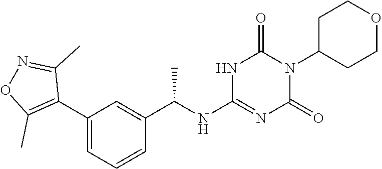 [SMILES: Cc1noc(C)c1-c1cccc([C@H](C)Nc2nc(=O)n(C3CCOCC3)c(=O)[nH]2)c1] | general procedure B, E | 412.2 |
| 199 | 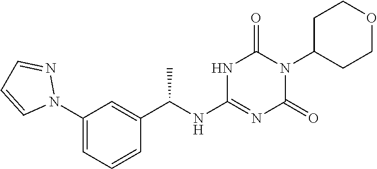 [SMILES: C[C@H](Nc1nc(=O)n(C2CCOCC2)c(=O)[nH]1)c1cccc(-n2cccn2)c1] | general procedure B, F | 383.4 |
| 200 | 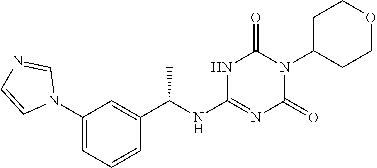 [SMILES: C[C@H](Nc1nc(=O)n(C2CCOCC2)c(=O)[nH]1)c1cccc(-n2ccnc2)c1] | general procedure B, F | 383.2 |
| 201 | 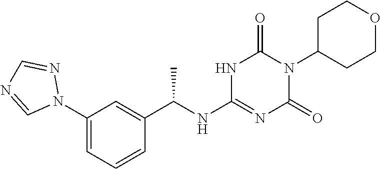 [SMILES: C[C@H](Nc1nc(=O)n(C2CCOCC2)c(=O)[nH]1)c1cccc(-n2cncn2)c1] | general procedure B, F | 384.2 |
| 202 | 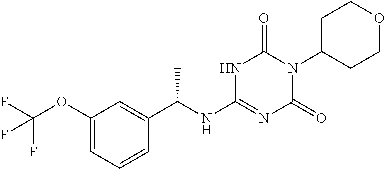 [SMILES: C[C@H](Nc1nc(=O)n(C2CCOCC2)c(=O)[nH]1)c1cccc(OC(F)(F)F)c1] | general procedure B Example 2 | 401.1 |
| 203 | 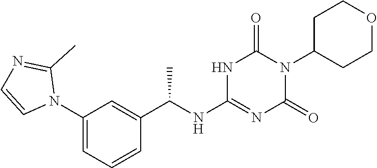 [SMILES: Cc1nccn1-c1cccc([C@H](C)Nc2nc(=O)n(C3CCOCC3)c(=O)[nH]2)c1] | general procedure B, F | 397.5 |
| 204 | 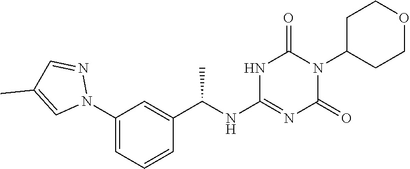 [SMILES: Cc1cnn(-c2cccc([C@H](C)Nc3nc(=O)n(C4CCOCC4)c(=O)[nH]3)c2)c1] | general procedure B, F | 397.2 |
| 205 | 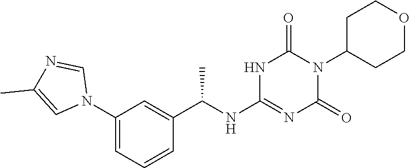 [SMILES: Cc1cn(-c2cccc([C@H](C)Nc3nc(=O)n(C4CCOCC4)c(=O)[nH]3)c2)cn1] | general procedure B, F | 397.2 |
| 206 | 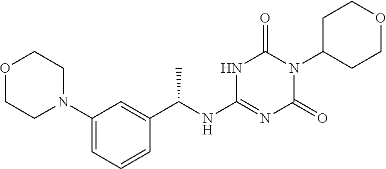 [SMILES: C[C@H](Nc1nc(=O)n(C2CCOCC2)c(=O)[nH]1)c1cccc(N2CCOCC2)c1] | general procedure B, F | 402.2 |
| 207 | 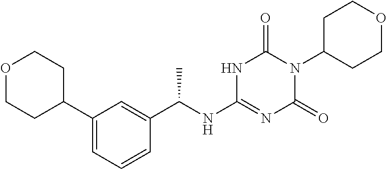 [SMILES: C[C@H](Nc1nc(=O)n(C2CCOCC2)c(=O)[nH]1)c1cccc(C2CCOCC2)c1] | general procedure B, E, G | 401.2 |
| 208 | 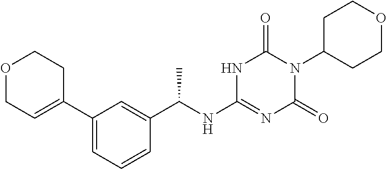 [SMILES: C[C@H](Nc1nc(=O)n(C2CCOCC2)c(=O)[nH]1)c1cccc(C2=CCOCC2)c1] | general procedure B, E | 399.2 |
| 209 | 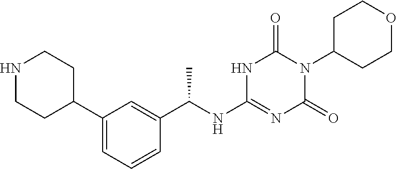 [SMILES: C[C@H](Nc1nc(=O)n(C2CCOCC2)c(=O)[nH]1)c1cccc(C2CCNCC2)c1] | general procedure B, E, G | 400.2 |
| 210 | 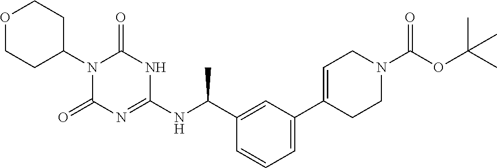 [SMILES: C[C@H](Nc1nc(=O)n(C2CCOCC2)c(=O)[nH]1)c1cccc(C2=CCN(C(=O)OC(C)(C)C)CC2)c1] | general procedure B, E | 498.3 |
| 211 | 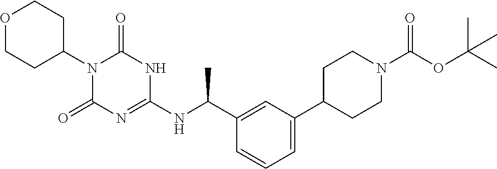 [SMILES: C[C@H](Nc1nc(=O)n(C2CCOCC2)c(=O)[nH]1)c1cccc(C2CCN(C(=O)OC(C)(C)C)CC2)c1] | general procedure B, E, G | 500.3 |
| 212 | 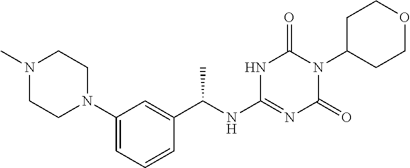 [SMILES: C[C@H](Nc1nc(=O)n(C2CCOCC2)c(=O)[nH]1)c1cccc(N2CCN(C)CC2)c1] | general procedure B, F | 415.2 |
| 213 | 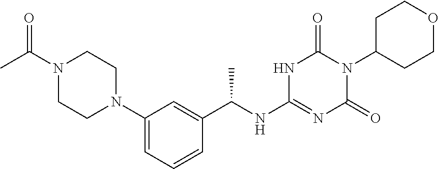 [SMILES: CC(=O)N1CCN(c2cccc([C@H](C)Nc3nc(=O)n(C4CCOCC4)c(=O)[nH]3)c2)CC1] | general procedure B, F | 443.2 |
| 214 | 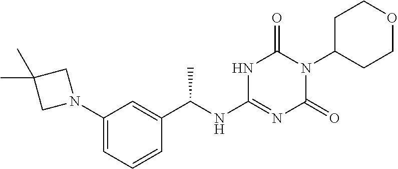 [SMILES: C[C@H](Nc1nc(=O)n(C2CCOCC2)c(=O)[nH]1)c1cccc(N2CC(C)(C)C2)c1] | general procedure B, F | 400.2 |
| 215 | 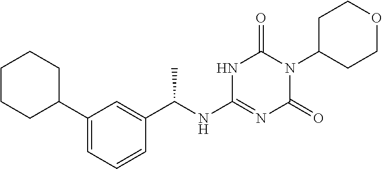 [SMILES: C[C@H](Nc1nc(=O)n(C2CCOCC2)c(=O)[nH]1)c1cccc(C2CCCCC2)c1] | general procedure B, E, G | 399.2 |
| 216 | 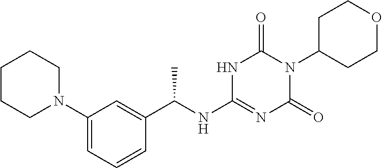 [SMILES: C[C@H](Nc1nc(=O)n(C2CCOCC2)c(=O)[nH]1)c1cccc(N2CCCCC2)c1] | general procedure B, F | 400.2 |
| 217 | 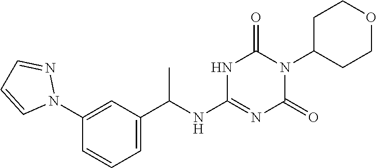 [SMILES: CC(Nc1nc(=O)n(C2CCOCC2)c(=O)[nH]1)c1cccc(-n2cccn2)c1] | general procedure B, F | 383.4 |
| 218 | 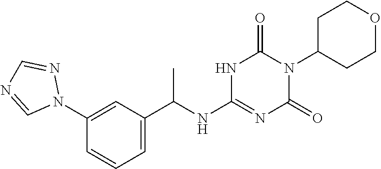 [SMILES: CC(Nc1nc(=O)n(C2CCOCC2)c(=O)[nH]1)c1cccc(-n2cncn2)c1] | general procedure B, F | 384.4 |
| 219 | 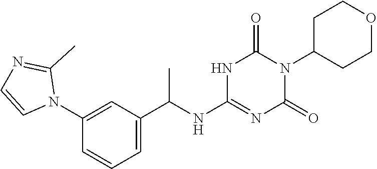 [SMILES: Cc1nccn1-c1cccc(C(C)Nc2nc(=O)n(C3CCOCC3)c(=O)[nH]2)c1] | general procedure B, F | 397.5 |
| 220 | 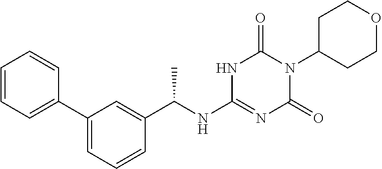 [SMILES: C[C@H](Nc1nc(=O)n(C2CCOCC2)c(=O)[nH]1)c1cccc(-c2ccccc2)c1] | general procedure B, E | 393.2 |
| 221 | 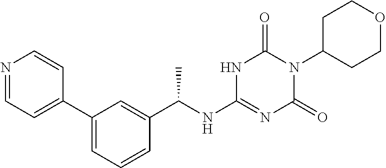 [SMILES: C[C@H](Nc1nc(=O)n(C2CCOCC2)c(=O)[nH]1)c1cccc(-c2ccncc2)c1] | general procedure B, E | 394.2 |
| 222 | 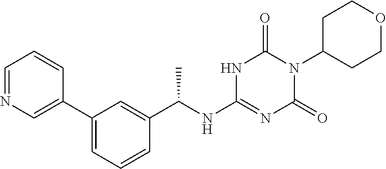 [SMILES: C[C@H](Nc1nc(=O)n(C2CCOCC2)c(=O)[nH]1)c1cccc(-c2cccnc2)c1] | general procedure B, E | 394.2 |
| 223 | 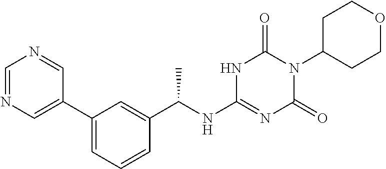 [SMILES: C[C@H](Nc1nc(=O)n(C2CCOCC2)c(=O)[nH]1)c1cccc(-c2cncnc2)c1] | general procedure B, E | 395.2 |
| 224 | 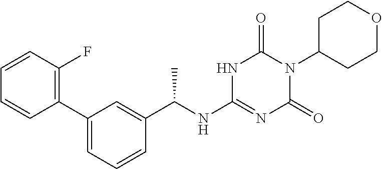 [SMILES: C[C@H](Nc1nc(=O)n(C2CCOCC2)c(=O)[nH]1)c1cccc(-c2ccccc2F)c1] | general procedure B, E | 411.2 |
| 225 | 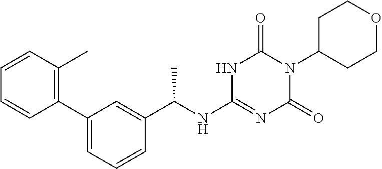 [SMILES: Cc1ccccc1-c1cccc([C@H](C)Nc2nc(=O)n(C3CCOCC3)c(=O)[nH]2)c1] | general procedure B, E | 407.5 |
| 226 | 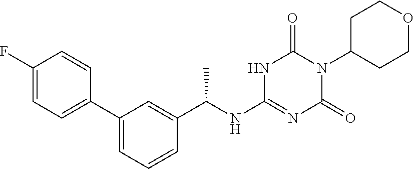 [SMILES: C[C@H](Nc1nc(=O)n(C2CCOCC2)c(=O)[nH]1)c1cccc(-c2ccc(F)cc2)c1] | general procedure B, E | 411.2 |
| 227 | 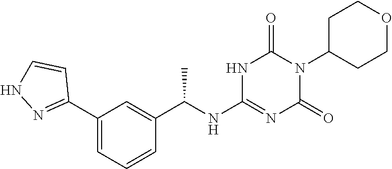 [SMILES: C[C@H](Nc1nc(=O)n(C2CCOCC2)c(=O)[nH]1)c1cccc(-c2cc[nH]n2)c1] | general procedure B, E | 383.2 |
| 228 | 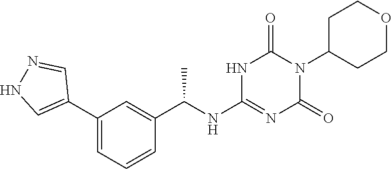 [SMILES: C[C@H](Nc1nc(=O)n(C2CCOCC2)c(=O)[nH]1)c1cccc(-c2cn[nH]c2)c1] | general procedure B, E | 383.2 |
| 229 | 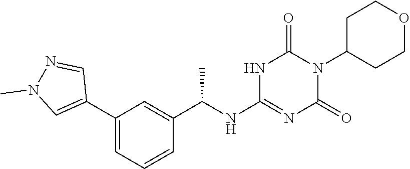 [SMILES: C[C@H](Nc1nc(=O)n(C2CCOCC2)c(=O)[nH]1)c1cccc(-c2cnn(C)c2)c1] | general procedure B, E | 397.2 |
| 230 | 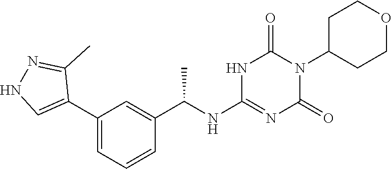 [SMILES: Cc1n[nH]cc1-c1cccc([C@H](C)Nc2nc(=O)n(C3CCOCC3)c(=O)[nH]2)c1] | general procedure B, E | 397.2 |
| 231 | 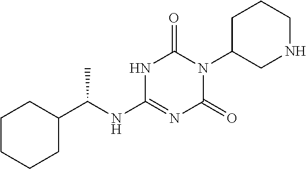 [SMILES: C[C@H](Nc1nc(=O)n(C2CCCNC2)c(=O)[nH]1)C1CCCCC1] | general procedure B, C | 322.2 |
| 232 | 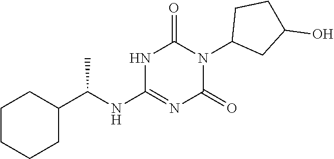 [SMILES: C[C@H](Nc1nc(=O)n(C2CCC(O)C2)c(=O)[nH]1)C1CCCCC1] | general procedure B, D | 323.4 |
| 233 | 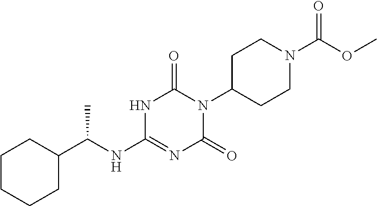 [SMILES: COC(=O)N1CCC(n2c(=O)nc(N[C@@H](C)C3CCCCC3)[nH]c2=O)CC1] | general procedure B, C | 380.2 |
| 234 | 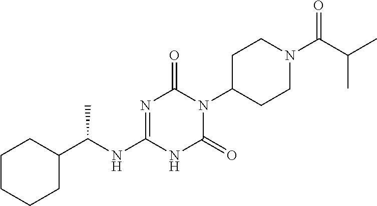 [SMILES: CC(C)C(=O)N1CCC(n2c(=O)nc(N[C@@H](C)C3CCCCC3)[nH]c2=O)CC1] | general procedure B, C | 392.5 |
| 235 | 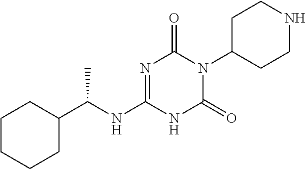 [SMILES: C[C@H](Nc1nc(=O)n(C2CCNCC2)c(=O)[nH]1)C1CCCCC1] | general procedure B, C | 322.4 |
| 236 | 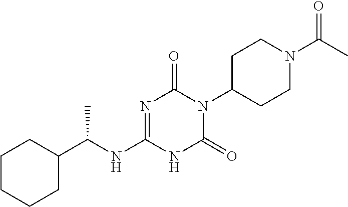 [SMILES: CC(=O)N1CCC(n2c(=O)nc(N[C@@H](C)C3CCCCC3)[nH]c2=O)CC1] | general procedure B, C | 364.5 |
| 237 | 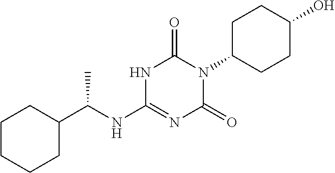 [SMILES: C[C@H](Nc1nc(=O)n([C@H]2CC[C@@H](O)CC2)c(=O)[nH]1)C1CCCCC1] | general procedure B, D | 337.4 |
| 238 | 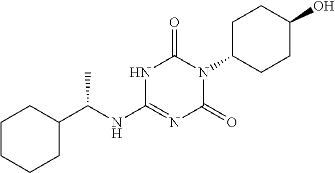 [SMILES: C[C@H](Nc1nc(=O)n([C@H]2CC[C@H](O)CC2)c(=O)[nH]1)C1CCCCC1] | general procedure B, D | 337.4 |
| 239 | 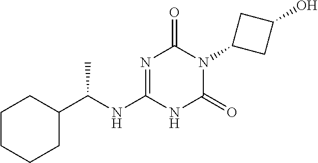 [SMILES: C[C@H](Nc1nc(=O)n([C@H]2C[C@@H](O)C2)c(=O)[nH]1)C1CCCCC1] | general procedure B, D | 309.0 |
| 240 | 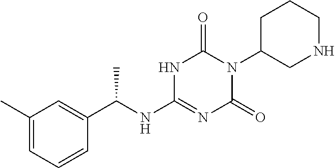 [SMILES: Cc1cccc([C@H](C)Nc2nc(=O)n(C3CCCNC3)c(=O)[nH]2)c1] | general procedure B, C | 330.4 |
| 241 | 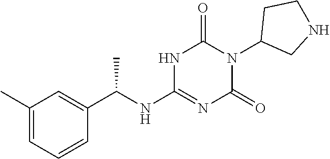 [SMILES: Cc1cccc([C@H](C)Nc2nc(=O)n(C3CCNC3)c(=O)[nH]2)c1] | general procedure B, C | 316.2 |
| 242 | 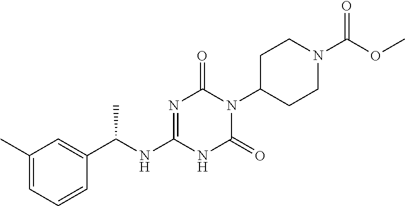 | general procedure B, C | 388.4 |
| 243 | 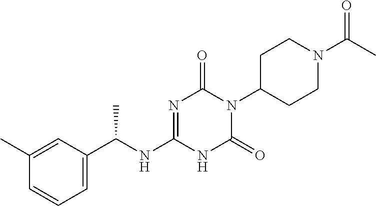 [SMILES: CC(=O)N1CCC(n2c(=O)nc(N[C@@H](C)c3cccc(C)c3)[nH]c2=O)CC1] | general procedure B, C | 372.4 |
| 244 | 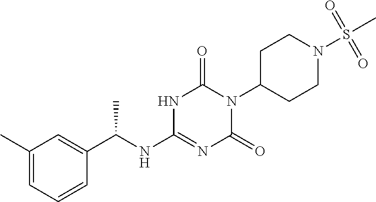 | general procedure B, C | 408.5 |
| 245 | 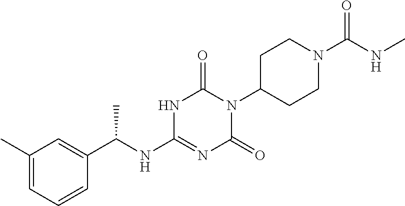 | general procedure B, C | 387.5 |
| 246 | 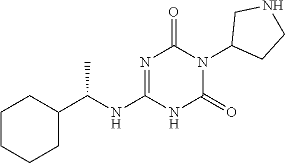 [SMILES: C[C@H](Nc1nc(=O)n(C2CCNC2)c(=O)[nH]1)C1CCCCC1] | general procedure B, C | 308.4 |
| 247 | 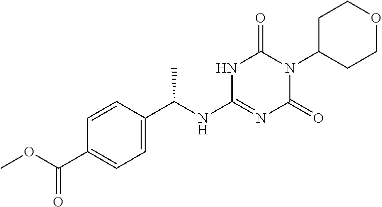 [SMILES: COC(=O)c1ccc([C@H](C)Nc2nc(=O)n(C3CCOCC3)c(=O)[nH]2)cc1] | general procedure B Example 2 | 375.2 |
| 248 | 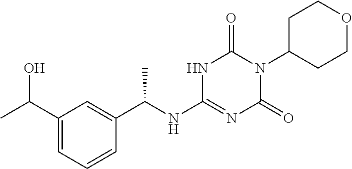 [SMILES: CC(O)c1cccc([C@H](C)Nc2nc(=O)n(C3CCOCC3)c(=O)[nH]2)c1] | general procedure B Example 9 | 361.2 |
| 249 | 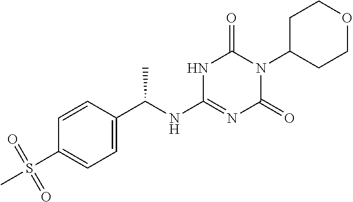 [SMILES: C[C@H](Nc1nc(=O)n(C2CCOCC2)c(=O)[nH]1)c1ccc(S(C)(=O)=O)cc1] | general procedure B Example 2 | 395.1 |
| 250 | 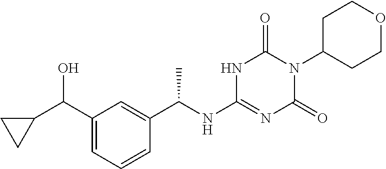 [SMILES: C[C@H](Nc1nc(=O)n(C2CCOCC2)c(=O)[nH]1)c1cccc(C(O)C2CC2)c1] | general procedure B Example 9 | 387.2 |
| 251 | 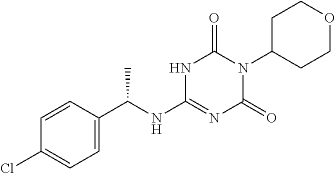 [SMILES: C[C@H](Nc1nc(=O)n(C2CCOCC2)c(=O)[nH]1)c1ccc(Cl)cc1] | general procedure B Example 2 | 351.1 |
| 252 | 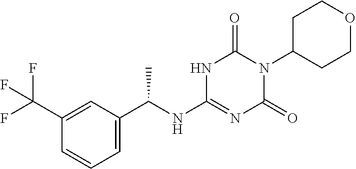 [SMILES: C[C@H](Nc1nc(=O)n(C2CCOCC2)c(=O)[nH]1)c1cccc(C(F)(F)F)c1] | general procedure B Example 2 | 385.4 |
| 253 | 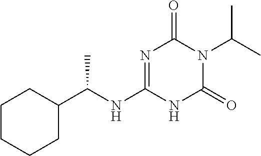 [SMILES: CC(C)n1c(=O)nc(N[C@@H](C)C2CCCCC2)[nH]c1=O] | general procedure B Example 2 | 281.4 |
| 254 | 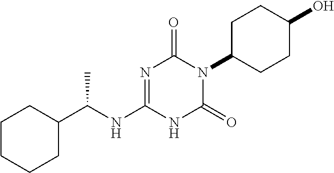 | general procedure B, D Example 2 | 337.4 |
| 255 | 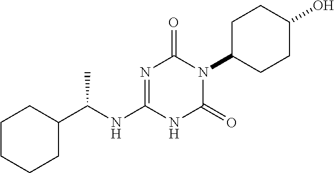 | general procedure B Example 2 | 337.4 |
| 256 | 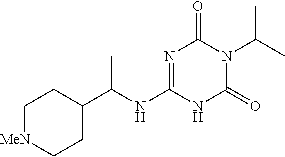 [SMILES: CC(Nc1nc(=O)n(C(C)C)c(=O)[nH]1)C1CCN(C)CC1] | general procedure B Example 2 | 296.2 |
| 257 | 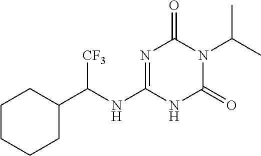 [SMILES: CC(C)n1c(=O)nc(NC(C2CCCCC2)C(F)(F)F)[nH]c1=O] | general procedure B Example 2 | 335.2 |
| 258 | 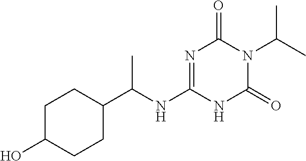 [SMILES: CC(Nc1nc(=O)n(C(C)C)c(=O)[nH]1)C1CCC(O)CC1] | general procedure B Example 2 | 297.2 |
| 259 | 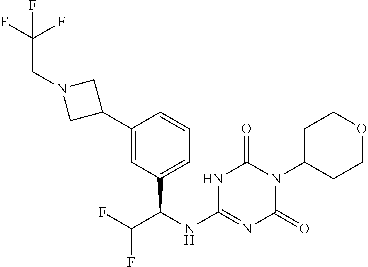 [SMILES: O=c1nc(N[C@H](c2cccc(C3CN(CC(F)(F)F)C3)c2)C(F)F)[nH]c(=O)n1C1CCOCC1] | general procedure B Example 2 | 490.2 |
| 260 | 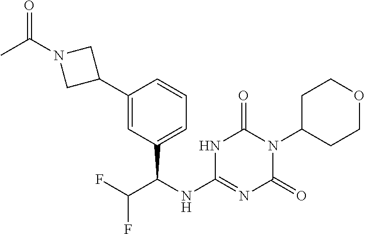 [SMILES: CC(=O)N1CC(c2cccc([C@@H](Nc3nc(=O)n(C4CCOCC4)c(=O)[nH]3)C(F)F)c2)C1] | general procedure B Example 2 | 450.2 |
| 261 | 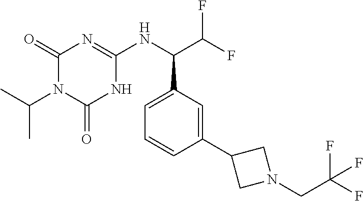 [SMILES: CC(C)n1c(=O)nc(N[C@H](c2cccc(C3CN(CC(F)(F)F)C3)c2)C(F)F)[nH]c1=O] | general procedure B, C | 448.2 |
| 262 | 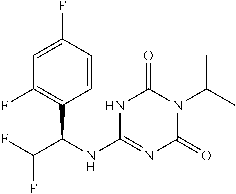 [SMILES: CC(C)n1c(=O)nc(N[C@H](c2ccc(F)cc2F)C(F)F)[nH]c1=O] | general procedure B, C | 347.1 |
| 263 | 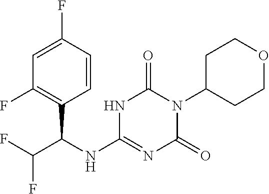 [SMILES: O=c1nc(N[C@H](c2ccc(F)cc2F)C(F)F)[nH]c(=O)n1C1CCOCC1] | general procedure B Example 2 | 389.1 |
| 264 | 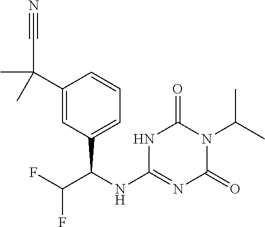 [SMILES: CC(C)n1c(=O)nc(N[C@H](c2cccc(C(C)(C)C#N)c2)C(F)F)[nH]c1=O] | general procedure B, C | 378.2 |
| 265 | 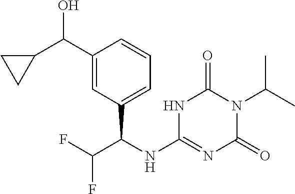 [SMILES: CC(C)n1c(=O)nc(N[C@H](c2cccc(C(O)C3CC3)c2)C(F)F)[nH]c1=O] | general procedure B, C | 381.2 |
| 266 | 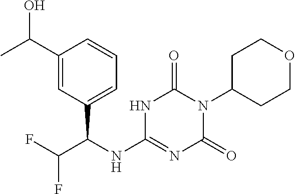 [SMILES: CC(O)c1cccc([C@@H](Nc2nc(=O)n(C3CCOCC3)c(=O)[nH]2)C(F)F)c1] | general procedure B Example 2 | 397.2 |
| 267 | 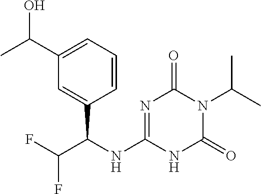 [SMILES: CC(O)c1cccc([C@@H](Nc2nc(=O)n(C(C)C)c(=O)[nH]2)C(F)F)c1] | general procedure B, C | 355.2 |
| 268 | 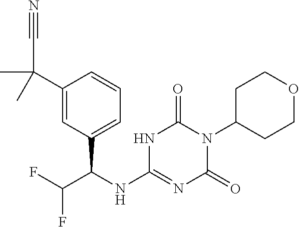 [SMILES: CC(C)(C#N)c1cccc([C@@H](Nc2nc(=O)n(C3CCOCC3)c(=O)[nH]2)C(F)F)c1] | general procedure B Example 2 | 420.2 |
| 269 | 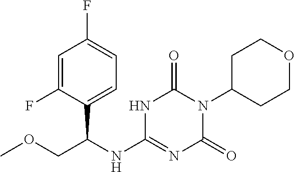 [SMILES: COC[C@H](Nc1nc(=O)n(C2CCOCC2)c(=O)[nH]1)c1ccc(F)cc1F] | general procedure B Example 2 | 383.2 |
| 270 | 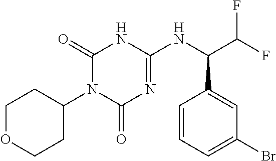 [SMILES: O=c1nc(N[C@H](c2cccc(Br)c2)C(F)F)[nH]c(=O)n1C1CCOCC1] | general procedure B Example 2 | 431.1 |
| 271 | 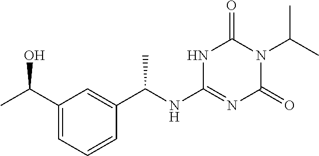 [SMILES: CC(C)n1c(=O)nc(N[C@@H](C)c2cccc([C@@H](C)O)c2)[nH]c1=O] | general procedure B, C | 319.2 |
| 272 | 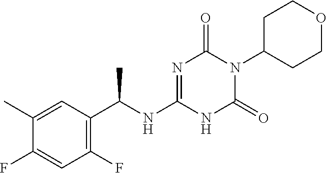 [SMILES: Cc1cc([C@@H](C)Nc2nc(=O)n(C3CCOCC3)c(=O)[nH]2)c(F)cc1F] | general procedure B Example 2 | 367.2 |
| 273 | 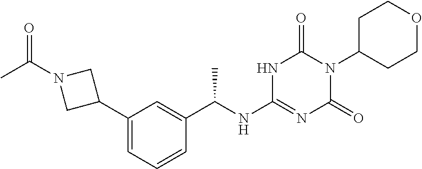 [SMILES: CC(=O)N1CC(c2cccc([C@H](C)Nc3nc(=O)n(C4CCOCC4)c(=O)[nH]3)c2)C1] | general procedure B Example 2 | 414.2 |
| 274 | 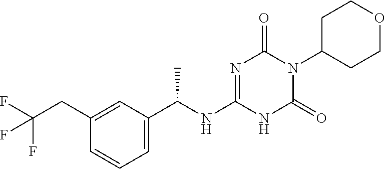 [SMILES: C[C@H](Nc1nc(=O)n(C2CCOCC2)c(=O)[nH]1)c1cccc(CC(F)(F)F)c1] | general procedure B Example 2 | 399.2 |
| 275 | 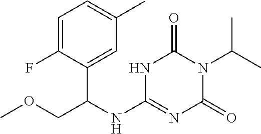 [SMILES: COCC(Nc1nc(=O)n(C(C)C)c(=O)[nH]1)c1cc(C)ccc1F] | general procedure B, C | 337.2 |
| 276 | 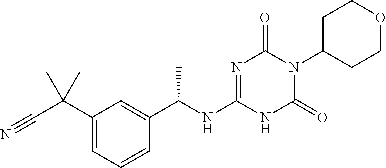 [SMILES: C[C@H](Nc1nc(=O)n(C2CCOCC2)c(=O)[nH]1)c1cccc(C(C)(C)C#N)c1] | general procedure B Example 2 | 384.2 |
| 277 | 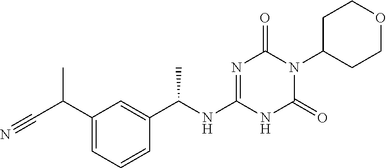 [SMILES: CC(C#N)c1cccc([C@H](C)Nc2nc(=O)n(C3CCOCC3)c(=O)[nH]2)c1] | general procedure B Example 2 | 370.2 |
| 278 | 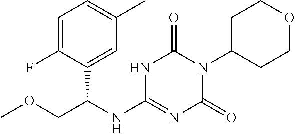 [SMILES: COC[C@@H](Nc1nc(=O)n(C2CCOCC2)c(=O)[nH]1)c1cc(C)ccc1F] | general procedure B Example 2 | 379.2 |
| 279 | 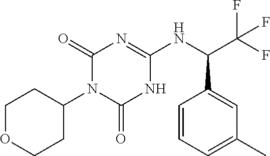 [SMILES: Cc1cccc([C@@H](Nc2nc(=O)n(C3CCOCC3)c(=O)[nH]2)C(F)(F)F)c1] | general procedure B Example 2 | 385.1 |
| 280 | 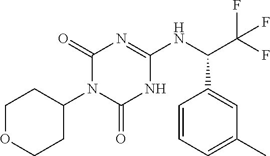 [SMILES: Cc1cccc([C@H](Nc2nc(=O)n(C3CCOCC3)c(=O)[nH]2)C(F)(F)F)c1] | general procedure B Example 2 | 385.1 |
| 281 | 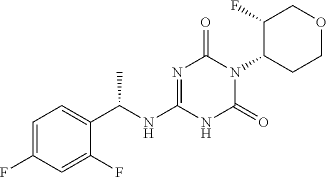 [SMILES: C[C@H](Nc1nc(=O)n([C@H]2CCOC[C@H]2F)c(=O)[nH]1)c1ccc(F)cc1F] | general procedure B Example 2 | 371.1 |
| 282 | 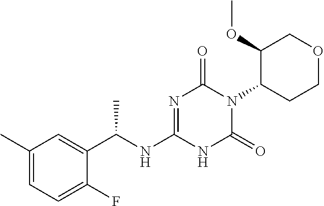 [SMILES: CO[C@H]1COCC[C@@H]1n1c(=O)nc(N[C@@H](C)c2cc(C)ccc2F)[nH]c1=O] | general procedure B Example 2 | 379.2 |
| 283 | 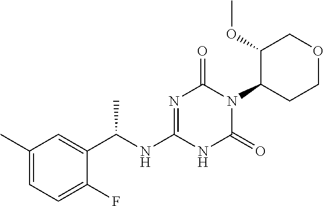 [SMILES: CO[C@@H]1COCC[C@H]1n1c(=O)nc(N[C@@H](C)c2cc(C)ccc2F)[nH]c1=O] | general procedure B Example 2 | 379.2 |
| 284 | 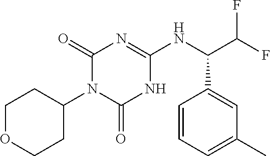 [SMILES: Cc1cccc([C@H](Nc2nc(=O)n(C3CCOCC3)c(=O)[nH]2)C(F)F)c1] | general procedure B Example 2 | 367.2 |
| 285 | 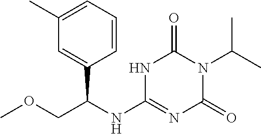 [SMILES: COC[C@H](Nc1nc(=O)n(C(C)C)c(=O)[nH]1)c1cccc(C)c1] | general procedure B, C | 319.2 |
| 286 | 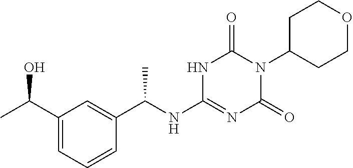 [SMILES: C[C@H](Nc1nc(=O)n(C2CCOCC2)c(=O)[nH]1)c1cccc([C@@H](C)O)c1] | general procedure B Example 2 | 361.2 |
| 287 | 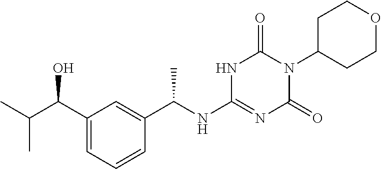 [SMILES: CC(C)[C@@H](O)c1cccc([C@H](C)Nc2nc(=O)n(C3CCOCC3)c(=O)[nH]2)c1] | general procedure B Example 2 | 389.2 |
| 288 | 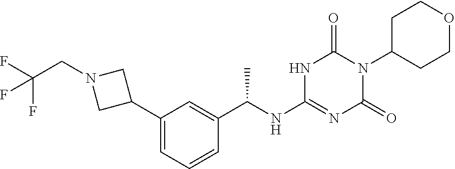 [SMILES: C[C@H](Nc1nc(=O)n(C2CCOCC2)c(=O)[nH]1)c1cccc(C2CN(CC(F)(F)F)C2)c1] | general procedure B Example 2 | 454.2 |
| 289 | 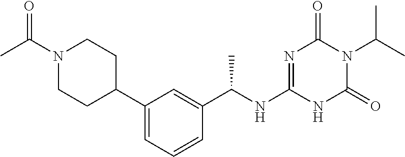 [SMILES: CC(=O)N1CCC(c2cccc([C@H](C)Nc3nc(=O)n(C(C)C)c(=O)[nH]3)c2)CC1] | general procedure B, C | 400.2 |
| 290 | 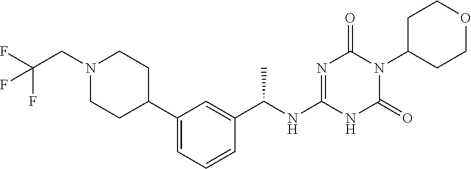 [SMILES: C[C@H](Nc1nc(=O)n(C2CCOCC2)c(=O)[nH]1)c1cccc(C2CCN(CC(F)(F)F)CC2)c1] | general procedure B Example 2 | 482.2 |
| 291 | 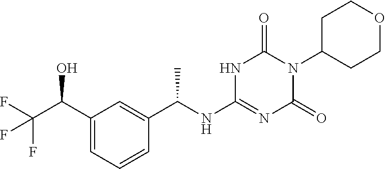 [SMILES: C[C@H](Nc1nc(=O)n(C2CCOCC2)c(=O)[nH]1)c1cccc([C@H](O)C(F)(F)F)c1] | general procedure B Example 2 | 415.2 |
| 292 | 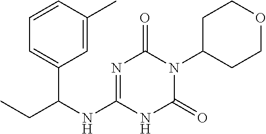 [SMILES: CCC(Nc1nc(=O)n(C2CCOCC2)c(=O)[nH]1)c1cccc(C)c1] | general procedure B Example 2 | 345.2 |
| 293 | 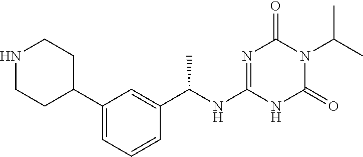 [SMILES: CC(C)n1c(=O)nc(N[C@@H](C)c2cccc(C3CCNCC3)c2)[nH]c1=O] | general procedure B, C | 358.2 |
| 294 | 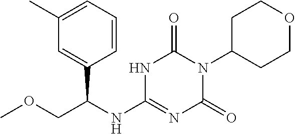 [SMILES: COC[C@H](Nc1nc(=O)n(C2CCOCC2)c(=O)[nH]1)c1cccc(C)c1] | general procedure B Example 2 | 361.2 |
| 295 | 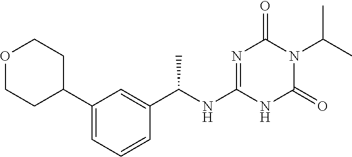 [SMILES: CC(C)n1c(=O)nc(N[C@@H](C)c2cccc(C3CCOCC3)c2)[nH]c1=O] | general procedure B, C | 359.2 |
| 296 | 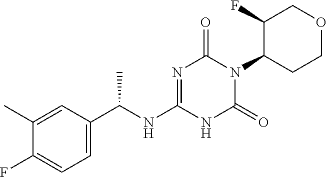 [SMILES: Cc1cc([C@H](C)Nc2nc(=O)n([C@@H]3CCOC[C@@H]3F)c(=O)[nH]2)ccc1F] | general procedure B Example 2 | 367.2 |
| 297 | 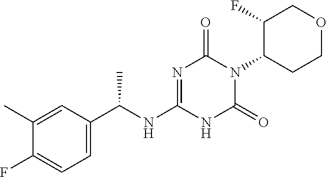 [SMILES: Cc1cc([C@H](C)Nc2nc(=O)n([C@H]3CCOC[C@H]3F)c(=O)[nH]2)ccc1F] | general procedure B Example 2 | 367.2 |
| 298 | 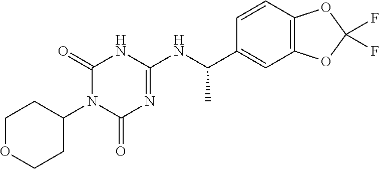 [SMILES: C[C@H](Nc1nc(=O)n(C2CCOCC2)c(=O)[nH]1)c1ccc2c(c1)OC(F)(F)O2] | general procedure B Example 2 | 397.1 |
| 299 | 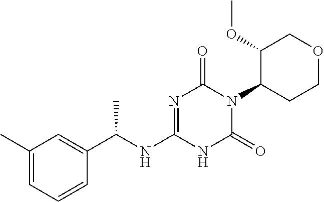 [SMILES: CO[C@@H]1COCC[C@H]1n1c(=O)nc(N[C@@H](C)c2cccc(C)c2)[nH]c1=O] | general procedure B Example 2 | 361.2 |
| 300 | 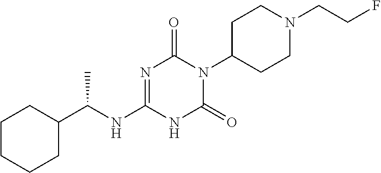 [SMILES: C[C@H](Nc1nc(=O)n(C2CCN(CCF)CC2)c(=O)[nH]1)C1CCCCC1] | general procedure B Example 2 | 368.2 |
| 301 | 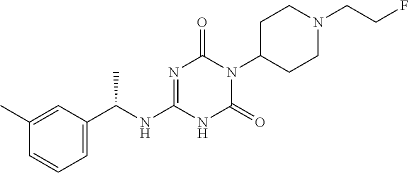 [SMILES: Cc1cccc([C@H](C)Nc2nc(=O)n(C3CCN(CCF)CC3)c(=O)[nH]2)c1] | general procedure B Example 2 | 376.2 |
| 302 | 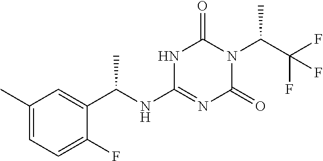 [SMILES: Cc1ccc(F)c([C@H](C)Nc2nc(=O)n([C@H](C)C(F)(F)F)c(=O)[nH]2)c1] | general procedure B Example 2 | 361.1 |
| 303 | 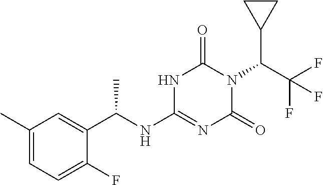 [SMILES: Cc1ccc(F)c([C@H](C)Nc2nc(=O)n([C@H](C3CC3)C(F)(F)F)c(=O)[nH]2)c1] | general procedure B Example 2 | 387.1 |
| 304 | 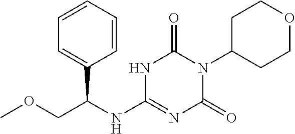 [SMILES: COC[C@H](Nc1nc(=O)n(C2CCOCC2)c(=O)[nH]1)c1ccccc1] | general procedure B Example 2 | 347.2 |
| 305 | 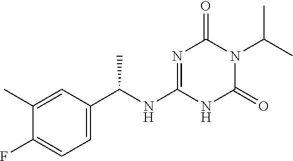 [SMILES: Cc1cc([C@H](C)Nc2nc(=O)n(C(C)C)c(=O)[nH]2)ccc1F] | general procedure B, C | 307.2 |
| 306 | 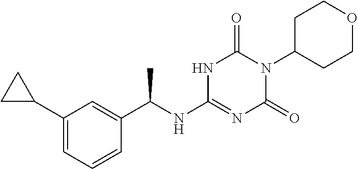 [SMILES: C[C@@H](Nc1nc(=O)n(C2CCOCC2)c(=O)[nH]1)c1cccc(C2CC2)c1] | general procedure B Example 2 | 357.2 |
| 307 | 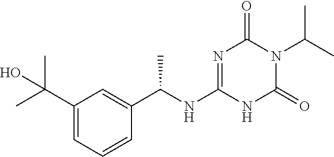 [SMILES: CC(C)n1c(=O)nc(N[C@@H](C)c2cccc(C(C)(C)O)c2)[nH]c1=O] | general procedure B, C | 333.2 |
| 308 | 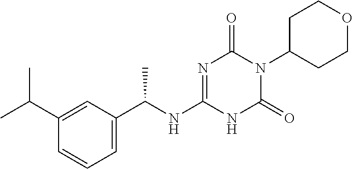 [SMILES: CC(C)c1cccc([C@H](C)Nc2nc(=O)n(C3CCOCC3)c(=O)[nH]2)c1] | general procedure B Example 2 | 359.2 |
| 309 | 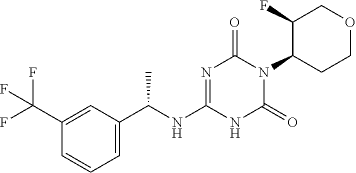 [SMILES: C[C@H](Nc1nc(=O)n([C@@H]2CCOC[C@@H]2F)c(=O)[nH]1)c1cccc(C(F)(F)F)c1] | general procedure B Example 2 | 403.1 |
| 310 | 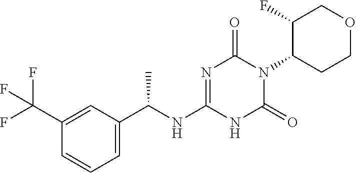 [SMILES: C[C@H](Nc1nc(=O)n([C@H]2CCOC[C@H]2F)c(=O)[nH]1)c1cccc(C(F)(F)F)c1] | general procedure B Example 2 | 403.1 |
| 311 | 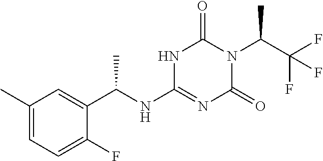 [SMILES: Cc1ccc(F)c([C@H](C)Nc2nc(=O)n([C@@H](C)C(F)(F)F)c(=O)[nH]2)c1] | general procedure B Example 2 | 361.1 |
| 312 | 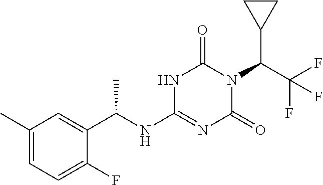 [SMILES: Cc1ccc(F)c([C@H](C)Nc2nc(=O)n([C@@H](C3CC3)C(F)(F)F)c(=O)[nH]2)c1] | general procedure B Example 2 | 387.1 |
| 313 | 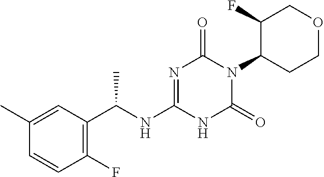 [SMILES: Cc1ccc(F)c([C@H](C)Nc2nc(=O)n([C@@H]3CCOC[C@@H]3F)c(=O)[nH]2)c1] | general procedure B Example 2 | 367.2 |
| 314 | 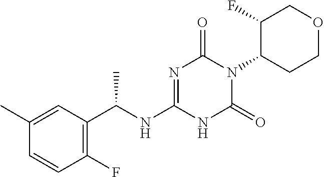 [SMILES: Cc1ccc(F)c([C@H](C)Nc2nc(=O)n([C@H]3CCOC[C@H]3F)c(=O)[nH]2)c1] | general procedure B Example 2 | 367.2 |
| 315 | 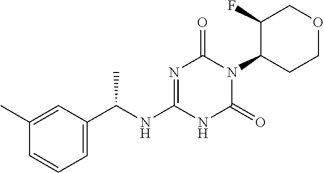 [SMILES: Cc1cccc([C@H](C)Nc2nc(=O)n([C@@H]3CCOC[C@@H]3F)c(=O)[nH]2)c1] | general procedure B Example 2 | 349.2 |
| 316 | 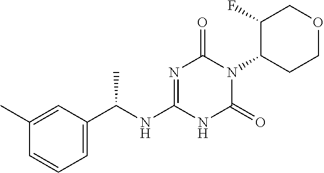 [SMILES: Cc1cccc([C@H](C)Nc2nc(=O)n([C@H]3CCOC[C@H]3F)c(=O)[nH]2)c1] | general procedure B Example 2 | 349.2 |
| 317 | 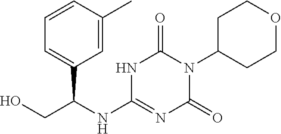 [SMILES: Cc1cccc([C@H](CO)Nc2nc(=O)n(C3CCOCC3)c(=O)[nH]2)c1] | general procedure B Example 2 | 347.2 |
| 318 | 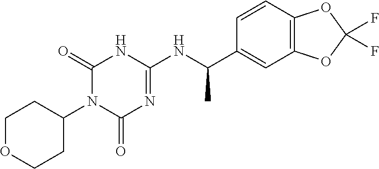 [SMILES: C[C@@H](Nc1nc(=O)n(C2CCOCC2)c(=O)[nH]1)c1ccc2c(c1)OC(F)(F)O2] | general procedure B Example 2 | 397.1 |
| 319 | 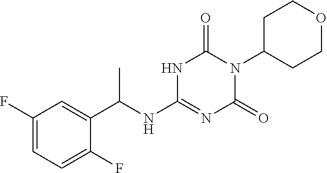 [SMILES: CC(Nc1nc(=O)n(C2CCOCC2)c(=O)[nH]1)c1cc(F)ccc1F] | general procedure B Example 2 | 353.1 |
| 320 | 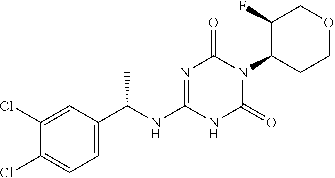 [SMILES: C[C@H](Nc1nc(=O)n([C@@H]2CCOC[C@@H]2F)c(=O)[nH]1)c1ccc(Cl)c(Cl)c1] | general procedure B Example 2 | 403.1 |
| 321 | 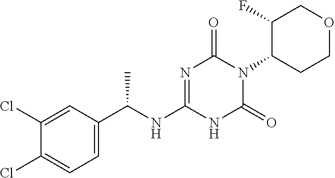 [SMILES: C[C@H](Nc1nc(=O)n([C@H]2CCOC[C@H]2F)c(=O)[nH]1)c1ccc(Cl)c(Cl)c1] | general procedure B Example 2 | 403.1 |
| 322 | 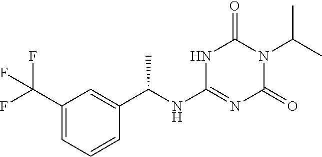 [SMILES: CC(C)n1c(=O)nc(N[C@@H](C)c2cccc(C(F)(F)F)c2)[nH]c1=O] | general procedure B, C | 343.1 |
| 323 | 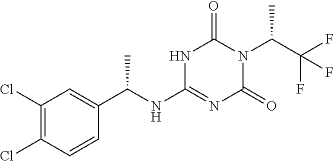 [SMILES: C[C@H](Nc1nc(=O)n([C@H](C)C(F)(F)F)c(=O)[nH]1)c1ccc(Cl)c(Cl)c1] | general procedure B Example 2 | 397.0 |
| 324 | 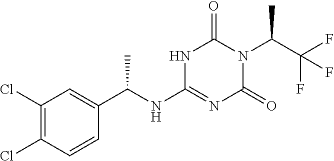 [SMILES: C[C@H](Nc1nc(=O)n([C@@H](C)C(F)(F)F)c(=O)[nH]1)c1ccc(Cl)c(Cl)c1] | general procedure B Example 2 | 397.0 |
| 325 | 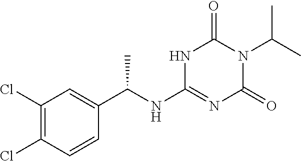 [SMILES: CC(C)n1c(=O)nc(N[C@@H](C)c2ccc(Cl)c(Cl)c2)[nH]c1=O] | general procedure B, C | 343.1 |
| 326 | 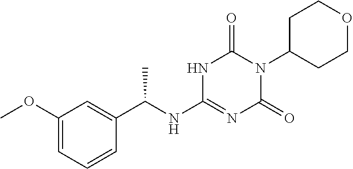 [SMILES: COc1cccc([C@H](C)Nc2nc(=O)n(C3CCOCC3)c(=O)[nH]2)c1] | general procedure B Example 2 | 347.2 |
| 327 | 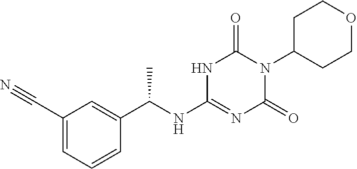 [SMILES: C[C@H](Nc1nc(=O)n(C2CCOCC2)c(=O)[nH]1)c1cccc(C#N)c1] | general procedure B Example 2 | 342.1 |
| 328 | 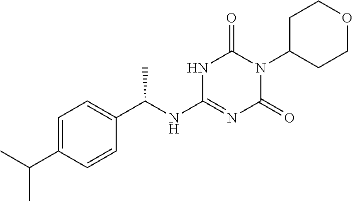 [SMILES: CC(C)c1ccc([C@H](C)Nc2nc(=O)n(C3CCOCC3)c(=O)[nH]2)cc1] | general procedure B Example 2 | 359.2 |
| 329 | 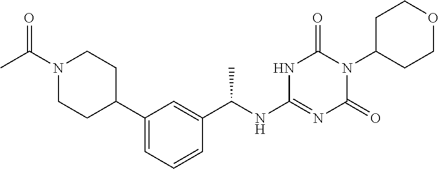 [SMILES: CC(=O)N1CCC(c2cccc([C@H](C)Nc3nc(=O)n(C4CCOCC4)c(=O)[nH]3)c2)CC1] | general procedure B Example 2 | 442.2 |
| 330 | 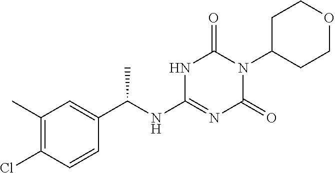 [SMILES: Cc1cc([C@H](C)Nc2nc(=O)n(C3CCOCC3)c(=O)[nH]2)ccc1Cl] | general procedure B Example 2 | 365.1 |
| 331 | 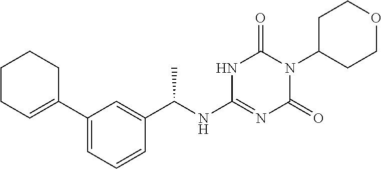 [SMILES: C[C@H](Nc1nc(=O)n(C2CCOCC2)c(=O)[nH]1)c1cccc(C2=CCCCC2)c1] | general procedure B Example 2 | 397.2 |
| 332 | 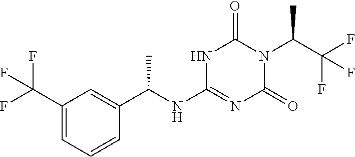 [SMILES: C[C@H](Nc1nc(=O)n([C@@H](C)C(F)(F)F)c(=O)[nH]1)c1cccc(C(F)(F)F)c1] | general procedure B Example 2 | 397.1 |
| 333 | 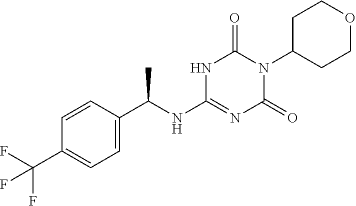 [SMILES: C[C@@H](Nc1nc(=O)n(C2CCOCC2)c(=O)[nH]1)c1ccc(C(F)(F)F)cc1] | general procedure B Example 2 | 385.1 |
| 334 | 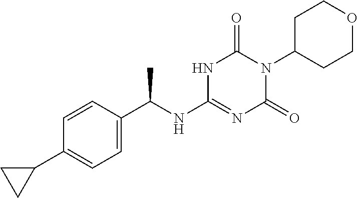 [SMILES: C[C@@H](Nc1nc(=O)n(C2CCOCC2)c(=O)[nH]1)c1ccc(C2CC2)cc1] | general procedure B Example 2 | 357.2 |
| 335 | 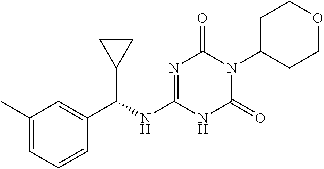 [SMILES: Cc1cccc([C@@H](Nc2nc(=O)n(C3CCOCC3)c(=O)[nH]2)C2CC2)c1] | general procedure B Example 2 | 357.2 |
| 336 | 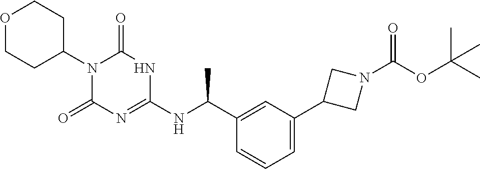 [SMILES: C[C@H](Nc1nc(=O)n(C2CCOCC2)c(=O)[nH]1)c1cccc(C2CN(C(=O)OC(C)(C)C)C2)c1] | general procedure B Example 2 | 472.3 |
| 337 | 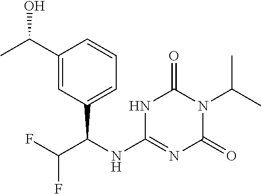 [SMILES: CC(C)n1c(=O)nc(N[C@H](c2cccc([C@H](C)O)c2)C(F)F)[nH]c1=O] | general procedure B, C | 355.2 |
| 338 | 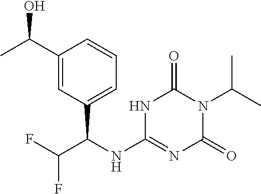 [SMILES: CC(C)n1c(=O)nc(N[C@H](c2cccc([C@@H](C)O)c2)C(F)F)[nH]c1=O] | general procedure B, C | 355.2 |
| 339 | 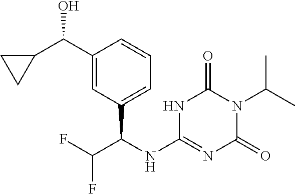 [SMILES: CC(C)n1c(=O)nc(N[C@H](c2cccc([C@@H](O)C3CC3)c2)C(F)F)[nH]c1=O] | general procedure B, C | 381.2 |
| 340 | 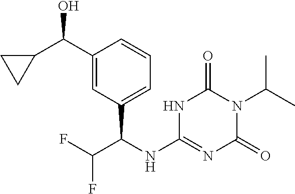 [SMILES: CC(C)n1c(=O)nc(N[C@H](c2cccc([C@H](O)C3CC3)c2)C(F)F)[nH]c1=O] | general procedure B, C | 381.2 |
| 341 | 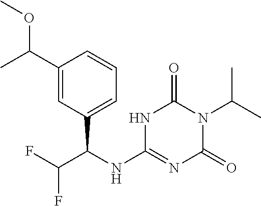 [SMILES: COC(C)c1cccc([C@@H](Nc2nc(=O)n(C(C)C)c(=O)[nH]2)C(F)F)c1] | general procedure B, C | 369.2 |
| 342 | 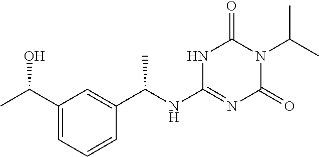 [SMILES: CC(C)n1c(=O)nc(N[C@@H](C)c2cccc([C@H](C)O)c2)[nH]c1=O] | general procedure B, C | 319.2 |
| 343 | 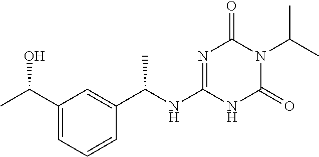 [SMILES: CC(C)n1c(=O)nc(N[C@@H](C)c2cccc([C@H](C)O)c2)[nH]c1=O] | general procedure B, C | 319.2 |
| 344 | 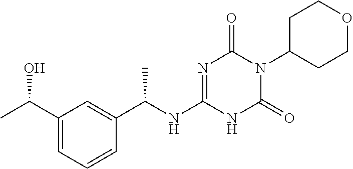 [SMILES: C[C@H](O)c1cccc([C@H](C)Nc2nc(=O)n(C3CCOCC3)c(=O)[nH]2)c1] | general procedure B Example 2 | 361.2 |
| 345 | 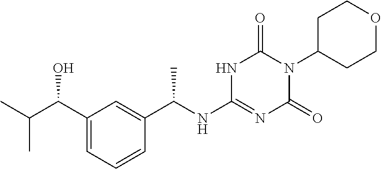 [SMILES: CC(C)[C@H](O)c1cccc([C@H](C)Nc2nc(=O)n(C3CCOCC3)c(=O)[nH]2)c1] | general procedure B Example 2 | 389.2 |
| 346 | 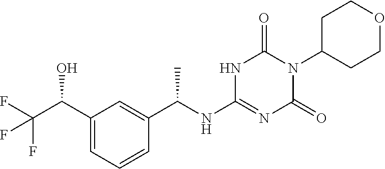 [SMILES: C[C@H](Nc1nc(=O)n(C2CCOCC2)c(=O)[nH]1)c1cccc([C@@H](O)C(F)(F)F)c1] | general procedure B Example 2 | 415.2 |
| 347 | 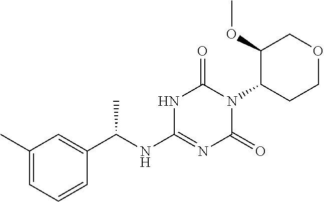 [SMILES: CO[C@H]1COCC[C@@H]1n1c(=O)nc(N[C@@H](C)c2cccc(C)c2)[nH]c1=O] | general procedure B Example 2 | 361.2 |
| 348 | 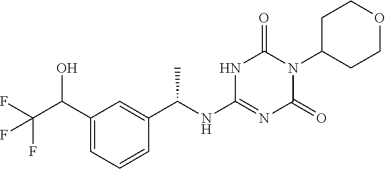 [SMILES: C[C@H](Nc1nc(=O)n(C2CCOCC2)c(=O)[nH]1)c1cccc(C(O)C(F)(F)F)c1] | general procedure B Example 2 | 415.2 |

### Biological Assays

### Experiment 1 Myosin Inhibitory Potency

As to the assays background, a biochemical assay couples the ATPase activity of bovine cardiac myosin to an enzymatic coupling system consisting of pyruvate kinase and lactate dehydrogenase (PK/LDH) and monitoring the absorbance decrease of NADH (at 340 nm) as a function of time to measure the inhibitory ability of small molecule agents. In the assay, PK converts ADP (Adenosine diphosphate) to ATP (adenosine triphosphate) by converting PEP (phosphoenolpyruvate) to pyruvate. Pyruvate is then converted to lactate by LDH by converting NADH (nicotinamide adenine dinucleotide) to NAD (oxidized nicotinamide adenine dinucleotide).

In our experiments, bovine skinned cardiac myofibrils were isolated from the frozen bovine left ventricle as myosin's source in the ATPase assay. The calcium concentration that achieves a 50% (pCa₅₀ or pCa=6.25) activation of the myofibril system was chosen as the final condition for assessing the activation activity according to the literature (DOI: 10.1074/jbc.M117.776815). Myofibrils ATPase activity was measured in a buffered solution containing 12 mM PIPES (piperazine-N, N′-bis(2-ethane sulfonic acid) and 2 mM magnesium chloride at pH 6.8 (PM12 buffer). Final assay conditions were 1 mg/mL of bovine cardiac myofibrils, 1:20 of stock PK/LDH (Sigma-Aldrich, Cat No. P0294-5X5ML), 50 μM ATP, 1 mM DTT (dithiothreitol), 0.75 mM NADH, 1.5 mM PEP at pCa₅₀ (pCa=6.25). Compounds were dissolved in DMSO (dimethyl sulfoxide). Serial dilution of compounds was created such that the final desired concentration of compound would be achieved in a volume of 150 μL with a fixed DMSO concentration of 2% (v/v). 75 μL of a solution containing bovine cardiac myofibrils, PK/LDH, and calcium were added to a 96 well plate for a 7 point dose-response. In some circumstances, 10 point-response was used to repeat the ATPase assays on compounds of interest. Compounds were added to the myofibrils solution and incubated for 5 minutes. The enzymatic reaction was started with the addition of 75 μL of a solution containing ATP, PEP, NADH, compounds, and calcium. The ATPase activity was measured by reading absorbance at 340 nm in a PerkinElmer Victor Nivo plate reader at 25° C. in kinetic mode for 15 minutes using clear bottom plates. The slopes of the absorbance changes as a function of time for the first 10 minutes were normalized to slopes on the control wells containing all reagents, including DMSO, but without compounds. This normalized rate was then plotted as a function of small molecule concentration in GraphPad prism 9. The data were fitted to a four-parameter fit, and IC₅₀ was calculated using Graphpad Prism 9. Any agent that failed to achieve the fifty percent inhibition at the highest concentration tested is reported as an IC₅₀ greater than the highest concentration tested (i.e., IC₅₀>200 uM).

TABLE 2
myosin inhibition activity

| Example # | IC₅₀ (uM) |
|---|---|
| 1 | 8.05 |
| 2 | 2.45 |
| 3 | 7.05 |
| 4 | 0.8 |
| 5 | 3.59 |
| 6 | 4.01 |
| 8 | 2.46 |
| 9 | 1.18 |
| 10 | 1.84 |
| 11 | 1.37 |
| 12 | 2.14 |
| 13 | 1.30 |
| 14 | 1.17 |
| 15 | 2.16 |
| 16 | 0.69 |
| 18 | 0.48 |
| 22 | 13.25 |
| 23 | 8.05 |
| 24 | 9.14 |
| 31 | 5.91 |
| 32 | 3.04 |
| 33 | 3.86 |
| 35 | 2.59 |
| 37 | 1.29 |
| 38 | 1.45 |
| 39 | 6.70 |
| 40 | 2.21 |
| 41 | 2.19 |
| 42 | 9.10 |
| 43 | 1.08 |
| 45 | 2.80 |
| 46 | 3.90 |
| 48 | 1.13 |
| 51 | 11.14 |
| 52 | 6.14 |
| 54 | 4.52 |
| 55 | 4.63 |
| 56 | 1.49 |
| 57 | 7.40 |
| 59 | 2.59 |
| 60 | 7.41 |
| 65 | 4.23 |
| 66 | 10.21 |
| 69 | 8.43 |
| 70 | 5.28 |
| 75 | 6.12 |
| 78 | 4.89 |
| 84 | 5.77 |
| 86 | 1.83 |
| 87 | 0.45 |
| 89 | 8.64 |
| 90 | 8.72 |
| 92 | 7.47 |
| 93 | 8.03 |
| 94 | 1.75 |
| 95 | 7.32 |
| 96 | 4.58 |
| 97 | 1.29 |
| 99 | 9.35 |
| 100 | 5.41 |
| 101 | 10.62 |
| 103 | 14.14 |
| 109 | 4.58 |
| 110 | 9.00 |
| 114 | 14.22 |
| 116 | 3.95 |
| 118 | 13.00 |
| 119 | 3.40 |
| 120 | 1.58 |
| 121 | 2.62 |
| 122 | 3.48 |
| 124 | 1.74 |
| 125 | 8.40 |
| 126 | 1.89 |
| 127 | 1.83 |
| 128 | 1.88 |
| 129 | 0.23 |
| 130 | 4.70 |
| 131 | 2.49 |
| 133 | 0.24 |
| 134 | 3.64 |
| 136 | 4.98 |
| 137 | 4.93 |
| 139 | 6.65 |
| 140 | 12.30 |
| 141 | 6.79 |
| 142 | 4.83 |
| 143 | 7.64 |
| 145 | 3.77 |
| 146 | 5.51 |
| 148 | 1.14 |
| 149 | 5.50 |
| 150 | 6.63 |
| 151 | 1.77 |
| 152 | 1.21 |
| 153 | 1.23 |
| 154 | 0.37 |
| 156 | 1.13 |
| 157 | 1.00 |
| 158 | 0.52 |
| 159 | 0.47 |
| 160 | 1.60 |
| 161 | 1.05 |
| 162 | 1.21 |
| 163 | 0.93 |
| 164 | 1.36 |
| 165 | 0.20 |
| 166 | 5.37 |
| 168 | 1.70 |
| 169 | 1.45 |
| 170 | 1.52 |
| 171 | 0.91 |
| 172 | 0.47 |
| 173 | 2.85 |
| 174 | 2.82 |
| 175 | 2.76 |
| 176 | 7.90 |
| 177 | 1.75 |
| 178 | 3.32 |
| 179 | 3.62 |
| 181 | 2.38 |
| 186 | 0.74 |
| 187 | 3.34 |
| 188 | 4.41 |
| 189 | 6.72 |
| 190 | 4.65 |
| 191 | 7.63 |
| 192 | 5.81 |
| 193 | 5.15 |
| 194 | 4.47 |
| 196 | 2.67 |
| 197 | 2.04 |
| 198 | 1.21 |
| 199 | 2.10 |
| 200 | 1.78 |
| 201 | 4.85 |
| 202 | 0.49 |
| 203 | 2.07 |
| 204 | 5.43 |
| 205 | 3.84 |
| 206 | 3.80 |
| 207 | 1.32 |
| 208 | 1.6 |
| 209 | 1.95 |
| 210 | 3.30 |
| 211 | 1.31 |
| 212 | 17.71 |
| 213 | 7.79 |
| 214 | 3.4 |
| 215 | 0.88 |
| 216 | 1.30 |
| 217 | 3.66 |
| 219 | 3.38 |
| 220 | 1.32 |
| 221 | 2.53 |
| 222 | 2.05 |
| 223 | 0.89 |
| 224 | 0.70 |
| 225 | 0.82 |
| 226 | 1.75 |
| 227 | 1.67 |
| 228 | 4.14 |
| 229 | 0.82 |
| 230 | 5.71 |
| 232 | 8.35 |
| 234 | 7.08 |
| 235 | 1.66 |
| 236 | 2.65 |
| 237 | 4.78 |
| 238 | 4.28 |
| 242 | 1.23 |
| 243 | 0.88 |
| 244 | 1.23 |
| 245 | 5.50 |
| 247 | 2.42 |
| 248 | 1.50 |
| 249 | 0.78 |
| 250 | 4.03 |
| 251 | 0.95 |
| 252 | 6.89 |
| 259 | 0.45 |
| 260 | 0.2 |
| 261 | 0.76 |
| 262 | 2.96 |
| 263 | 2.31 |
| 264 | 1.37 |
| 265 | 1.1 |
| 266 | 1.98 |
| 267 | 0.41 |
| 268 | 1.07 |
| 269 | 1.77 |
| 270 | 0.55 |
| 271 | 0.87 |
| 272 | 0.37 |
| 273 | 0.58 |
| 274 | 0.39 |
| 275 | 1.81 |
| 276 | 0.15 |
| 277 | 0.24 |
| 278 | 0.63 |
| 279 | 0.72 |
| 280 | 2.43 |
| 281 | 2.26 |
| 282 | 0.9 |
| 283 | 1.09 |
| 284 | 0.29 |
| 285 | 0.51 |
| 286 | 0.24 |
| 287 | 0.54 |
| 288 | 0.36 |
| 289 | 1.8 |
| 290 | 2.21 |
| 291 | 0.85 |
| 292 | 0.91 |
| 293 | 2.81 |
| 294 | 0.88 |
| 295 | 1.36 |
| 296 | 0.86 |
| 297 | 1.59 |
| 298 | 0.88 |
| 299 | 1.18 |
| 300 | 2.29 |
| 301 | 0.8 |
| 302 | 0.88 |
| 303 | 0.56 |
| 304 | 1.78 |
| 305 | 0.95 |
| 306 | 1.22 |
| 307 | 1.87 |
| 308 | 1 |
| 309 | 1.61 |
| 310 | 1.49 |
| 311 | 2.36 |
| 312 | 1.44 |
| 313 | 1.37 |
| 314 | 0.77 |
| 315 | 1.47 |
| 316 | 1.23 |
| 317 | 2.85 |
| 318 | 1.05 |
| 319 | 1.28 |
| 320 | 1.02 |
| 321 | 1.07 |
| 322 | 1.21 |
| 323 | 0.71 |
| 324 | 1.06 |
| 325 | 1.17 |
| 326 | 1.35 |
| 327 | 1.27 |
| 328 | 0.85 |
| 329 | 1.24 |
| 330 | 1.08 |
| 331 | 1.11 |
| 332 | 1.39 |
| 333 | 1.03 |
| 334 | 0.7 |
| 335 | 0.62 |
| 336 | 1.16 |
| 342 | 2.07 |
| 343 | 1.51 |
| 344 | 0.55 |
| 345 | 0.79 |
| 346 | 2.58 |
| 347 | 2.33 |
| 348 | 2.45 |

### Experiment 2. Myosin Inhibitory Potency Comparison in Cardiac and Skeletal Myofibrils

Bovine skinned cardiac myofibrils were isolated from the frozen bovine left ventricle, and rabbit skinned skeletal myofibrils were isolated from the frozen rabbit Psoas major and minor muscles as myosin's source in the ATPase assay. The calcium concentration that achieves a 50% activation of the myofibril system (pCa=6.25 for bovine cardiac myofibrils and pCa=6 for rabbit skeletal myofibrils) was chosen as the final condition for assessing the activation activity according to the literature (DOI:10.1074/jbc.M117.776815). Rest of ATPase assay conditions are the same as illustrated in experiment 1.

TABLE 3
Myosin inhibition activity
comparison in cardiac and skeletal myofibrils

| Example # | Skeletal IC₅₀ | Cardiac IC₅₀ | Skeleta/Cardiac |
|---|---|---|---|
| 4 | 1.80 | 0.66 | 2.72 |
| 10 | 43.40 | 1.50 | 29.01 |
| 45 | 40.6 | 7.58 | 5.36 |
| 152 | 4.27 | 1.79 | 2.39 |

Compounds of the invention show great potency on cardiac myofibrils. Additionally, Example 10 is way less potent in inhibiting fast skeletal myofibril activity. The data confirmed that Example 10 has better cardiac-skeletal myosin selectivity thus could lead to better safety profile.

### Experiment 3. Cardiomyocyte Contractility Assay

The effects of compounds on sarcomere shortening in isolated rat ventricular myocytes were assessed using the IonOptix apparatus.

Myocytes were placed in a chamber mounted on the stage of an inverted microscope and continuously superfused with oxygenated Tyrode solution containing (in millimolar): 121 NaCl, 5 KCl, 2.8 NaCH₃CO₂, MgCl₂·6H₂O, Glucose, NaHCO₃, Na₂HPO₄. 7H₂O, and 1.5 mM CaCl₂). Solution was preheated at 36±1° C. and electrical-field stimulated at 1 Hz by 2 platinum electrodes connected to a Myopacer field stimulator (IonOptix Corporation) with 4 ms square-wave bipolar pulses (10 V). Cells were illuminated by the microscope light. The cell image was collected by a x40 ultraviolet epifluorescence objective, diverted to the microscope side port, where the cell image was recorded by a charge coupled device (CCD) camera (MyoCam, IonOptix Corporation), converting optical brightness (pixels) into electrical signals (voltage). The MyoCam configuration allowed acquisition of up to 240 images per second (240 Hz frame rate). Contractile properties of the myocytes were analyzed in real time by a video detector and a personal computer-based data acquisition system (Ionwizard 6.0, IonOptix Corporation). Only myocytes with clear striations, quiescent prior to pacing with a resting sarcomere length greater or equal to 1.75 μm were used, since this is presumed to represent the lower limit for healthy cells.

Sarcomere shortening was monitored in control solution (predrug) until stable recordings were obtained (baseline period). To determine the response to compounds, myocytes were first superfused for 60 seconds with Tyrode's buffer followed by at least a 5 minute—(or until steady state was reached, up to 10 min) superfusion of compound. Each cell was subjected to 2 concentrations (5 and 15 uM or 5 and 10 uM) of test compounds. For some cells, a washout period was performed after the last concentration. Duration of the washout period was variable, resulting in variability in the washout data. In separate cells, a single concentration of isoproterenol (100 nM) was applied. Data were continuously recorded using the IonOptix software. Contractility data were analyzed using Ionwizard software (IonOptix). For each cell, 10-15 contractility transients at baseline and after treatment were averaged and compared.

TABLE 4
The effect of the compounds on
fractional shortening of the myocytes

| | | % FS | % FS | Ratio |
|---|---|---|---|---|
| | | (% reduction | (% reduction | of % FS |
| | # of | from | from baseline) | 5 uM/ |
| Example | cells | baseline) | at 10 uM* | 10 or |
| # | tested | at 5 uM | or 15uM | 15 uM |
| 4 | 3 | 71.2 +/− 7.6 | 42.4 +/− 4.2* | 1.7* |
| 10 | 4 | 51 +/− 6 | 41.5 +/− 6.3 | 1.2 |
| 152 | 3 | 62.9 +/− 6.2 | 26.7 +/− 6.4 | 2.4 |
| 156 | 3 | 61.5 +/− 5.2 | 26.1 +/− 4 | 2.4 |
| 162 | 3 | 60.8 +/− 12.7 | 32.2 +/− 5.3 | 1.9 |
| 168 | 3 | 54.3 +/− 9.3 | 24.3 +/− 8.5 | 2.2 |
| 172 | 3 | 63.6 +/− 3.3 | 28.9 +/− 8.7 | 2.2 |

*means % FS at 10 uM, others % FS at 15 uM.
Ratio of fractional shortening at 5 uM/10 or 15 uM indicates the responsiveness of the myocyte contractility to compound treatment. A lower ratio suggests compounds of the invention may have a higher therapeutic window in vivo.

### Experiment 4. Pharmacokinetic Profiles

Pharmacokinetic profile of compounds were determined by IV (1 mg/Kg) and PO (5 mg/Kg) administrations in male SD rats. Compounds were administrated with free base and formulated in 5% DMAC+25% PEG-400+70%(30% 2-HP-β-CD in water). The compounds were dosed at 1 mg/kg for intravenous and 5 mg/kg oral administration. Blood samples were collected at 0, 0.083, 0.25, 0.5, 1, 2, 4, 8 and 24 hours post dose, serial bleeding for plasma for the IV group. Blood samples were collected at 0, 0.25, 0.5, 1, 2, 4, 6, 8 and 24 hours post dose, serial bleeding for the PO group. Approximately 150 μL whole blood/time point were collected in K2EDTA tube via jugular vein. Blood sample was put on ice and centrifuged at 2000 g for 5 min to obtain plasma sample within 15 minutes. PK parameters were estimated by non-compartmental model using WinNonlin 8.2.

TABLE 5
Pharmacokinetic parameters
of the examples in male SD rats

| Example | CL | VSS | T1/2 | DNAUC |
|---|---|---|---|---|
| # | (L/hr/kg) | (L/kg) | (hr) | (hr*ng/ml) |
| 4 | 0.33 | 1.17 | 3.35 | 2299.6 |
| 10 | 0.55 | 1.78 | 4.51 | 1724.7 |
| 156 | 0.19 | 0.97 | 5.47 | 5052.2 |
| 162 | 0.25 | 0.97 | 3.74 | 4195.1 |
| 165 | 0.1 | 0.46 | 4.58 | 11463.4 |
| 168 | 0.08 | 0.32 | 3.89 | 13412.2 |
| 172 | 0.15 | 0.57 | 3.95 | 7631.0 |
| 252 | 0.21 | 0.94 | 4.41 | 3959.8 |

Compounds of the invention generally showed shorter half-life. This could be an advantage as shorter half-life could reduce the time to reach equilibrium at steady state. It can also reduce or avoid clinical accumulation of drugs in the body and avoid the risks caused by accumulation.

### Experiment 5. Echocardiography Assessment of Acute Pharmacodynamic Effect in Rat Cardiac Contractility

The Effect of Compounds on Heart Function was Determined by Echocardiography in Spraw-Dawley rats. Rats were under light anesthesia with 1-2% isoflurane. Compounds were dosed via oral gavage as single PO. Baseline heart functions were measured 1 day before dosing. The effect of compounds on heart function were measured at 1, 3, 6, and 24 hours post dosing. About 250 μL of whole blood was obtained at ˜1, 3, 6 and 24 hours post-dose via tail vein, immediately after the Echocardiography procedure. Blood was placed into a plasma separator tube containing K2 EDTA and kept on wet ice until processing. Blood samples were centrifuged at 2,000 g (4,400 rpm, Eppendorf 5417R) for 10 minutes at 4° C. Plasma samples were then transferred into micro-tubes and stored at −80° C. for future LC/MS analysis. The data were plotted as reduction of Fractional shortening vs plasma compound concentration. Therapeutic windows were determined as IC₅₀/IC₁₀ according to the literature (DOI: https://doi.org/10.1021/acs.jmedchem.1c01290).

FIG. 2 shows that Example 10 is dose dependent under 10 mpk and do not further inhibit heart contractility at 20 mpk. This result indicates that Example 10 has better safety profile compare to other compounds tested. Data plotted with plasma exposure (PK) vs fractional shortening (OD, FS % to baseline) also confirmed that Example 10 has superior therapeutic windows with shallower slope of the curve.

1. A compound of formula (I): 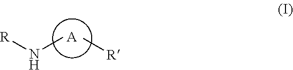
or a tautomer, cis- or trans-isomer, mesomer, racemate, enantiomer, diastereomer, or mixture thereof, or a pharmaceutically acceptable salt, solvate, or prodrug thereof, wherein:
A is selected from the group consisting of: 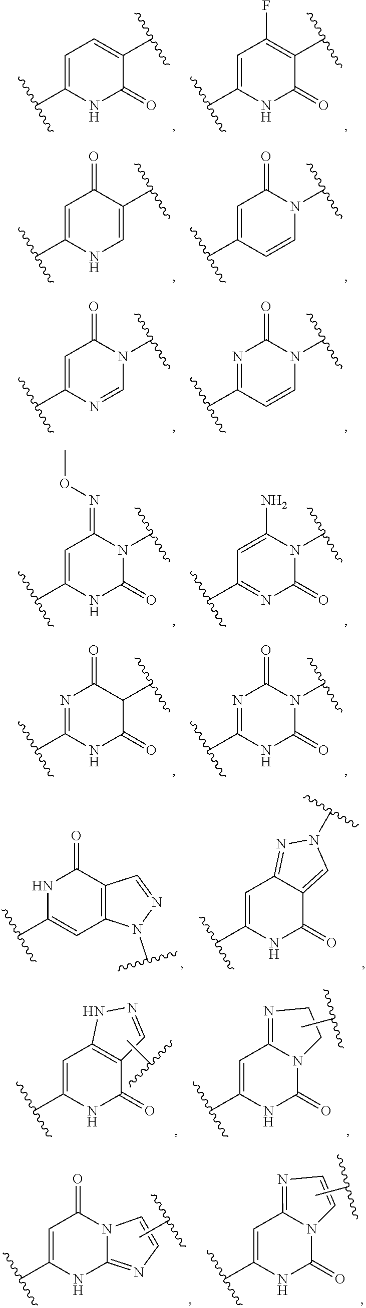 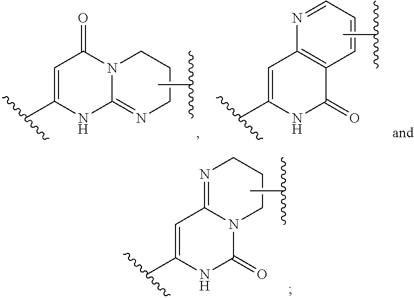
R is —(CR₁R₂)ₙR₃;
R₁ and R₂ are independently selected from the group consisting of hydrogen, deuterium, halogen, amino, cyano, hydroxyl, alkyl, alkoxy, haloalkyl, hydroxyalkyl, cycloalkyl, heterocyclyl, aryl and heteroaryl;
n is 0, 1, 2, 3 or 4;
R₃ is selected from the group consisting of hydrogen, deuterium, halogen, amino, cyano, hydroxyl, alkyl, alkoxy, haloalkyl, hydroxyalkyl, cycloalkyl, heterocyclyl, aryl and heteroaryl, wherein each of alkyl, alkoxy, haloalkyl, hydroxyalkyl, cycloalkyl, heterocyclyl, aryl and heteroaryl at each occurrence is independently unsubstituted or substituted with one or more substituents selected from the R³ group consisting of deuterium, halogen, amino, nitro, oxo, cyano, hydroxy, alkyl, alkoxy, haloalkyl, hydroxyalkyl, —NRₐR_{b}, —C(O)Rₐ, —C(O)NRₐR_{b}, —C(O)ORₐ, —OC(O)Rₐ, —S(O)ₘRₐ, —S(O)ₘNRₐR_{b}, cycloalkyl, heterocyclyl, aryl and heteroaryl, wherein the alkyl, hydroxyalkyl, cycloalkyl, heterocyclyl, aryl, and heteroaryl in said R³ group of substituents is independently unsubstituted or substituted with one or more substituents selected from alkyl, haloalkyl, cyano, —C(O)Rₐ, halogen, and cycloalkyl;
m is 0, 1 or 2;
R′ is selected from the group consisting of alkyl, alkoxy, haloalkyl, hydroxyalkyl, cycloalkyl, heterocyclyl, aryl and heteroaryl, wherein each of alkyl, alkoxy, haloalkyl, hydroxyalkyl, cycloalkyl, heterocyclyl, aryl and heteroaryl at each occurrence is independently unsubstituted or substituted with one or more substituents selected from the group consisting of deuterium, halogen, amino, cyano, hydroxy, alkyl, alkoxy, haloalkyl, hydroxyalkyl, —NR_{c}R_{d}, —C(O)R_{c}, —C(O)NR_{c}R_{d}, —C(O)OR_{c}, —OC(O)R_{c},
—S(O)ₘR_{c} and —S(O)ₘNR_{c}R_{d};
Rₐ, R_{b}, R_{c}, and R_{d} are independently selected from the group consisting of hydrogen, deuterium, halogen, amino, cyano, hydroxy, alkyl, alkoxy, haloalkyl and hydroxyalkyl.

2. The compound of claim 1, or a tautomer, cis- or trans-isomer, mesomer, racemate, enantiomer, diastereomer, or mixture thereof, or a pharmaceutically acceptable salt, solvate, or prodrug thereof, being a compound of formula (II): 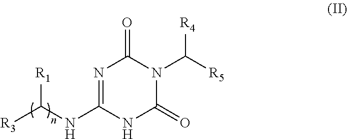
wherein,
R₁ is selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl;
R₃ is selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₁₀ cycloalkyl, 4-10 membered heterocyclyl comprising one or more of the members of N, O, S and S(O)₂, C₆-C₁₂ aryl and 4-10 membered heteroaryl comprising one or more of the members of N, O, S and S(O)₂, wherein each of the C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl at each occurrence is independently unsubstituted or substituted with one to four substituents selected from the R³ group consisting of deuterium, halogen, amino, cyano, hydroxyl, C₁-C₆ alkyl, C₂-C₆ alkenyl, C₂-C₆ alkynyl, C₁-C₆ haloalkyl, C₁-C₆ alkoxy, C₁-C₆ haloalkoxy, C₁-C₆ hydroxyalkyl, C₃-C₆ cycloalkyl, 4-6 membered heterocyclyl comprising one or more of the members of N, O, S and S(O)₂, phenyl, 4-6 membered heteroaryl comprising one or more of the members of N, O, S and S(O)₂, —NRₐR_{b}, —C(O)Rₐ, —C(O)NRₐR_{b}, —C(O)ORₐ, —OC(O)Rₐ, —S(O)ₘRₐ, —S(O)ₘNRₐR_{b} and —OSiRₐR_{b}R_{c}, wherein the C₃-C₆ cycloalkyl, 4-6 membered heterocyclyl comprising one or more of the members of N, O, S and S(O)₂, phenyl, 4-6 membered heteroaryl comprising one or more of the members of N, O, S and S(O)₂, C₁-C₆ alkyl, and C₁-C₆ hydroxyalkyl in said R³ group of substituents is independently unsubstituted or substituted with one or more substituents selected from C₁-C₆ alkyl, C₁-C₆ haloalkyl, cyano, —C(O)Rₐ, halogen, and C₃-C₆ cycloalkyl;
R₄ and R₅ are independently selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl comprising one or more of the members of N, O, S and S(O)₂, C₆-C₁₂ aryl and 4-8 membered heteroaryl comprising one or more of the members of N, O, S and S(O)₂, wherein each of the C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl at each occurrence is independently unsubstituted or substituted with one or more substituents selected from the group consisting of deuterium, halogen, amino, cyano, hydroxyl, C₁-C₆ alkyl, C₁-C₆ alkoxy, C₁-C₆ haloalkyl, C₁-C₆ hydroxyalkyl, —NR_{c}R_{d}, —C(O)R_{c}, —C(O)NR_{c}R_{d}, —C(O)OR_{c} and —OC(O)R_{c};
or, R₄ and R₅ together with the C atom to which they are bound form a cyclic structure selected from the R⁴⁵Cycle group consisting of C₃-C₈ cycloalkyl, 4-8 membered heterocyclyl comprising one or more of the members of N and O, C₆-C₁₂ aryl and 4-8 membered heteroaryl comprising one or more of the members of N and O, wherein each of the cyclic structures in said R⁴⁵Cycle group is optionally substituted with one to four substituents selected from the group consisting of deuterium, halogen, amino, cyano, hydroxyl, C₁-C₆ alkyl, C₁-C₆ haloalkyl, C₁-C₆ alkoxy, C₁-C₆ hydroxyalkyl, —NR_{c}R_{d}, —C(O)R_{c}, —C(O)NR_{c}R_{d}, —C(O)OR_{c} and —OC(O)R_{c};
Rₐ, R_{b}, R_{c}, and R_{d} are independently selected from the group consisting of hydrogen, deuterium, halogen, amino, cyano, hydroxyl, C₁-C₆ alkyl, C₁-C₆ alkoxy, C₁-C₆ haloalkyl and C₁-C₆ hydroxyalkyl.

3. The compound of claim 1, or a tautomer, cis- or trans-isomer, mesomer, racemate, enantiomer, diastereomer, or mixture thereof, or a pharmaceutically acceptable salt, solvate, or prodrug thereof, being a compound of formula (II): 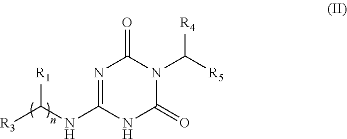
wherein,
R₁ is selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl;
R₃ is selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl comprising one or more of the members of N, O, S and S(O)₂, C₆-C₁₂ aryl and 4-8 membered heteroaryl comprising one or more of the members of N, O, S and S(O)₂, wherein the C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl, C₆-C₁₂ aryl and 4-8 membered heteroaryl heteroaryl at each occurrence is independently unsubstituted or substituted with one or more substituents selected from the R³ group consisting of deuterium, halogen, amino, nitro, oxo, cyano, hydroxyl, C₁-C₆ alkyl, C₁-C₆ alkoxy, C₁-C₆ haloalkyl, C₁-C₆ hydroxyalkyl, —NRₐR_{b}, —C(O)Rₐ, —C(O)NRₐR_{b}, —C(O)ORₐ, —OC(O)Rₐ, —S(O)ₘRₐ and —S(O)ₘNRₐR_{b}, wherein the 4-6 membered heterocyclyl comprising one or more of the members of N, O, S and S(O)₂, C₁-C₆alkyl, C₁-C₆ hydroxyalkyl in said R³ group of substituents is independently unsubstituted or substituted with one or more substituents selected from C₁-C₆ alkyl, C₁-C₆ haloalkyl, cyano, —C(O)Rₐ, halogen, and C₃-C₆cycloalkyl;
R₄ and R₅ are independently selected from the group consisting of hydrogen, deuterium, halogen, amino, cyano, hydroxyl, C₁-C₆ alkyl, C₁-C₆ alkoxy, C₁-C₆ haloalkyl, C₁-C₆ hydroxyalkyl, C₃-C₈ cycloalkyl, 4-8 membered heterocyclyl comprising one or more of the members of N, O, S and S(O)₂, C₆-C₁₂ aryl and 4-8 membered heteroaryl comprising one or more of the members of N, O, S and S(O)₂, wherein the alkyl, alkoxy, haloalkyl, hydroxyalkyl, cycloalkyl, heterocyclyl, aryl and heteroaryl at each occurrence is independently unsubstituted or substituted with one or more substituents selected from the group consisting of deuterium, halogen, amino, cyano, hydroxyl, C₁-C₆ alkyl, C₁-C₆ alkoxy, C₁-C₆ haloalkyl, C₁-C₆ hydroxyalkyl, —NR_{c}R_{d}, —C(O)R_{c}, —C(O)NR_{c}R_{d}, —C(O)OR_{c} and —OC(O)R_{c};
or, R₄ and R₅ together with the C atom to which they are bound form a cyclic structure selected from the R⁴⁵Cycle group consisting of C₃-C₈ cycloalkyl, 4-8 membered heterocyclyl comprising N or O atom, C₆-C₁₂ aryl and 4-8 membered heteroaryl comprising N or O atom, wherein each of the cyclic structures in said R⁴⁵Cycle group is optionally substituted with one to four substituents selected from the group consisting of by deuterium, halogen, amino, cyano, hydroxyl, C₁-C₆ alkyl, C₁-C₆ haloalkyl, C₁-C₆ alkoxy, C₁-C₆ hydroxyalkyl, —NR_{c}R_{d}, —C(O)R_{c}, —C(O)NR_{c}R_{d}, —C(O)OR_{c} and —OC(O)R_{c};
Rₐ, R_{b}, R_{c}, and R_{d} are independently selected from the group consisting of hydrogen, deuterium, halogen, amino, cyano, hydroxyl, C₁-C₆ alkyl, C₁-C₆ alkoxy, C₁-C₆ haloalkyl and C₁-C₆ hydroxyalkyl.

4. The compound of claim 1, or a tautomer, cis- or trans-isomer, mesomer, racemate, enantiomer, diastereomer, or mixture thereof, or a pharmaceutically acceptable salt, solvate, or prodrug thereof, wherein, R₁ is selected from the group consisting of hydrogen, hydroxyl, C₁-C₃ alkyl, C₁-C₃ haloalkyl, C₁-C₃ alkoxy, and C₁-C₃ hydroxyalkyl.

5. The compound of claim 4, or a tautomer, cis- or trans-isomer, mesomer, racemate, enantiomer, diastereomer, or mixture thereof, or a pharmaceutically acceptable salt, solvate, or prodrug thereof, wherein, R₁ is H, —OH, —CH₃, —CH₂CH₃, —CH(CH₃)₂, —CH₂OH, —CF₃, or

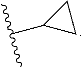 [SMILES: *C1CC1]

6. The compound of claim 1, or a tautomer, cis- or trans-isomer, mesomer, racemate, enantiomer, diastereomer, or mixture thereof, or a pharmaceutically acceptable salt, solvate, or prodrug thereof, wherein, R₃ is selected from the group consisting of C₁-C₃ alkyl, C₁-C₃ haloalkyl, C₁-C₃ alkoxy, C₁-C₃ hydroxyalkyl, C₃-C₆ cycloalkyl, phenyl, 5-6 membered heterocyclyl comprising 1-2 of the members of N, O, S and S(O)₂ atom and 5-6 membered heteroaryl comprising 1-2 of the members of N, O, S and S(O)₂ atom, optionally the R₃ is substituted with one to two substituents selected from the R³ group consisting of deuterium, halogen, amino, nitro, oxo, cyano, hydroxyl, C₁-C₃ alkyl, C₁-C₃ alkoxy, C₁-C₃ haloalkyl, C₁-C₃ hydroxyalkyl, 4-6 membered heterocyclyl comprising one or more of the members of N, O, S and S(O)₂, —C(O)Rₐ, —C(O)NRₐR_{b}, —S(O)₂Rₐ and —S(O)₂NRₐR_{b}, wherein the C₁-C₃ alkyl, C₁-C₃ hydroxyalkyl and 4-6 membered heterocyclyl comprising one or more of the members of N, O, S and S(O)₂, in said R³ group of substituents is independently unsubstituted or substituted with one or more substituents selected from C₁-C₃ alkyl, C₁-C₃ haloalkyl, cyano, —C(O)Rₐ, halogen, and C₃-C₆ cycloalkyl;
Rₐ and R_{b} are independently selected from the group consisting of hydrogen, deuterium, halogen, amino, cyano, hydroxyl, C₁-C₃ alkyl, C₁-C₃ alkoxy, C₁-C₃ haloalkyl and C₁-C₃ hydroxyalkyl.

7. The compound of claim 2, or a tautomer, cis- or trans-isomer, mesomer, racemate, enantiomer, diastereomer, or mixture thereof, or a pharmaceutically acceptable salt, solvate, or prodrug thereof, wherein, R₄ and R₅ are independently selected from the group consisting of hydrogen, deuterium, halogen, amino, cyano, hydroxyl, C₁-C₃ alkyl, C₁-C₃ alkoxy, C₁-C₃ haloalkyl, C₁-C₃ hydroxyalkyl, C₃-C₆ cycloalkyl, 5-6 membered heterocyclyl comprising 1-2 of the members of N, O, S and S(O)₂ atom, C₆-C₁₂ aryl and 5-6 membered heteroaryl comprising 1-2 of the members of N, O, S and S(O)₂ atom, wherein each of C₁-C₃ alkyl, C₁-C₃ alkoxy, C₁-C₃ haloalkyl, C₁-C₃ hydroxyalkyl, C₃-C₆ cycloalkyl, 5-6 membered heterocyclyl, C₆-C₁₂ aryl and 5-6 membered heteroaryl at each occurrence is independently unsubstituted or substituted with one or more substituents selected from the group consisting of deuterium, halogen, amino, cyano, hydroxyl, C₁-C₃ alkyl, C₁-C₃ alkoxy, C₁-C₃ haloalkyl, C₁-C₃ hydroxyalkyl, —NR_{c}R_{d}, —C(O)R_{c}, —C(O)NR_{c}R_{d}, —C(O)OR_{c} and —OC(O)R_{c};
or, R₄ and R₅ together with the C atom to which they are bound form a a cyclic structure selected from the C⁴⁵Cycle(II) group consisting of C₃-C₆ cycloalkyl, 5-6 membered heterocyclyl comprising 1-2 of the members of N and O atom, phenyl and 5-6 membered heteroaryl comprising 1-2 of the members of N and O atom, wherein each of the cyclic structures in said C⁴⁵Cycle(II) group is optionally substituted with one to two substituents selected from the group consisting of by deuterium, halogen, amino, cyano, hydroxyl, C₁-C₃ alkyl, C₁-C₃ haloalkyl, C₁-C₃ alkoxy, C₁-C₃ hydroxyalkyl, —NR_{c}R_{d}, —C(O)R_{c}, —C(O)NR_{c}R_{d}, —C(O)OR_{c} and —OC(O)R_{c};
R_{c} and R_{d} are independently selected from the group consisting of hydrogen, deuterium, halogen, amino, cyano, hydroxyl, C₁-C₃ alkyl, C₁-C₃ alkoxy, C₁-C₃ haloalkyl and C₁-C₃ hydroxyalkyl.

8. The compound of claim 2, or a tautomer, cis- or trans-isomer, mesomer, racemate, enantiomer, diastereomer, or mixture thereof, or a pharmaceutically acceptable salt, solvate, or prodrug thereof, wherein, R₄ and R₅ are independently selected from the group consisting of —CH₃ and —CF₃;
or, R₄ and R₅ together with the C atom to which they are bound form a cyclic structure selected from the RCycle group consisting of: 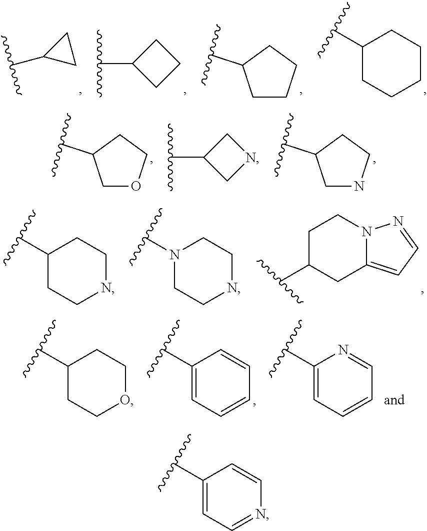
wherein each of the cyclic structures in said RCycle group is optionally substituted with one or two substituents selected from the group consisting of oxo, H, —F, —Cl, —Br, —OH, —CH₃, —CH₂CH₃, —CH(CH₃)₂, —OCH₃, —CH₂CH₃OCH₃, —CH₂OCH₃, —CF₃, —CH₂CF₃, —C(O)CH₃, —C(O)CH(CH₃)₂, —C(O)OCH₃, —C(O)OC(CH₃)₃, —NHC(O)OC(CH₃)₃, and

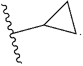 [SMILES: *C1CC1]

9. The compound of claim 1, or a tautomer, cis- or trans-isomer, mesomer, racemate, enantiomer, diastereomer, or mixture thereof, or a pharmaceutically acceptable salt, solvate, or prodrug thereof, wherein, n is 0, 1 or 2.

10. The compound of claim 2, or a tautomer, cis- or trans-isomer, mesomer, racemate, enantiomer, diastereomer, or mixture thereof, or a pharmaceutically acceptable salt, solvate, or prodrug thereof, wherein R₃ is 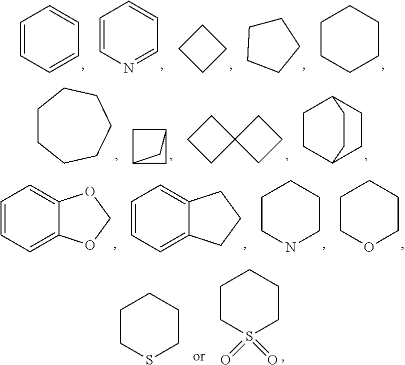
optionally R₃ is substituted with one or more substituents selected from the group consisting of deuterium, halogen, amino, nitro, oxo, cyano, hydroxyl, C₁-C₆ alkyl, C₁-C₆ alkoxy, C₁-C₆ haloalkyl, C₁-C₆ hydroxyalkyl, —NRₐR_{b}, —C(O)Rₐ, —C(O)NRₐR_{b}, —C(O)ORₐ, —OC(O)Rₐ, —S(O)ₘRₐ and —S(O)ₘNRₐR_{b}.

11. The compound of claim 2, or a tautomer, cis- or trans-isomer, mesomer, racemate, enantiomer, diastereomer, or mixture thereof, or a pharmaceutically acceptable salt, solvate, or prodrug thereof, being a compound of formula (III):

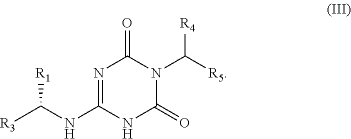

12. The compound of claim 2, or a tautomer, cis- or trans-isomer, mesomer, racemate, enantiomer, diastereomer, or mixture thereof, or a pharmaceutically acceptable salt, solvate, or prodrug thereof, wherein,
when each of R₄ and R₅ is methyl, n is 0, R₃ is not 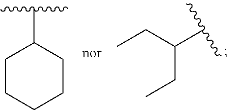
when each of R₁, R₄ and R₅ is methyl, n is 1, R₃ is not

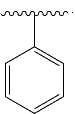 [SMILES: *c1ccccc1]

13. The compound of claim 11, or a tautomer, cis- or trans-isomer, mesomer, racemate, enantiomer, diastereomer, or mixture thereof, or a pharmaceutically acceptable salt, solvate, or prodrug thereof, being a compound of formula (IV): 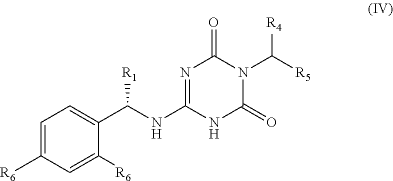
wherein,
R₁ is C₁-C₃ alkyl, C₁-C₃ haloalkyl or C₁-C₃ alkoxy;
R₄ and R₅ together with the C atom to which they are bound form a 5-6 membered heterocyclyl comprising 1-2 of of the members N and O;
R₆ is independently selected from the group consisting of halogen, C₁-C₃ alkyl, C₁-C₃ alkoxy, and C₁-C₃ haloalkyl.

14. A compound selected from the group consisting of: 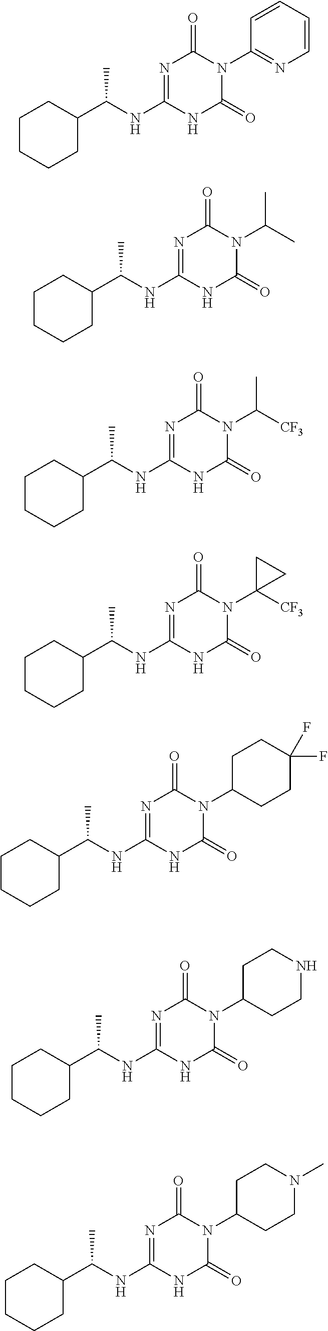 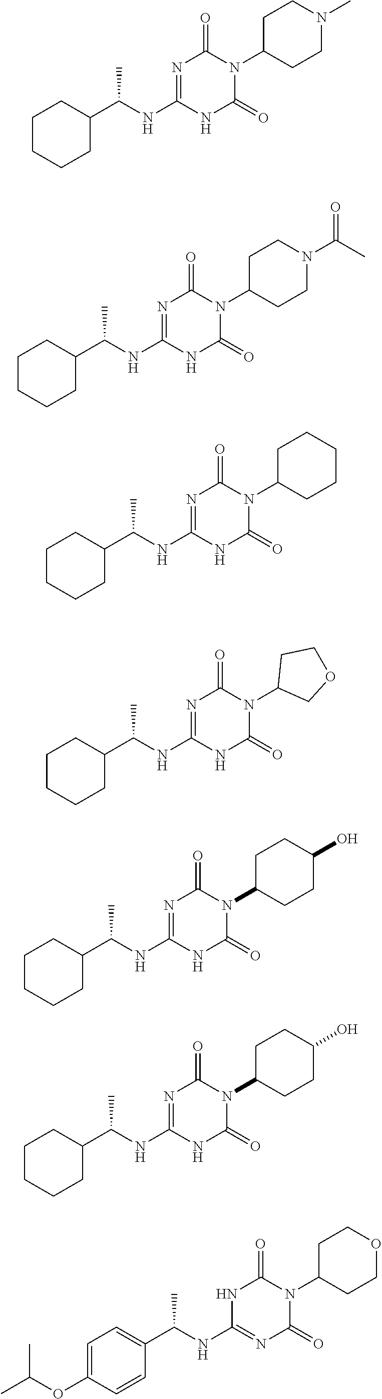 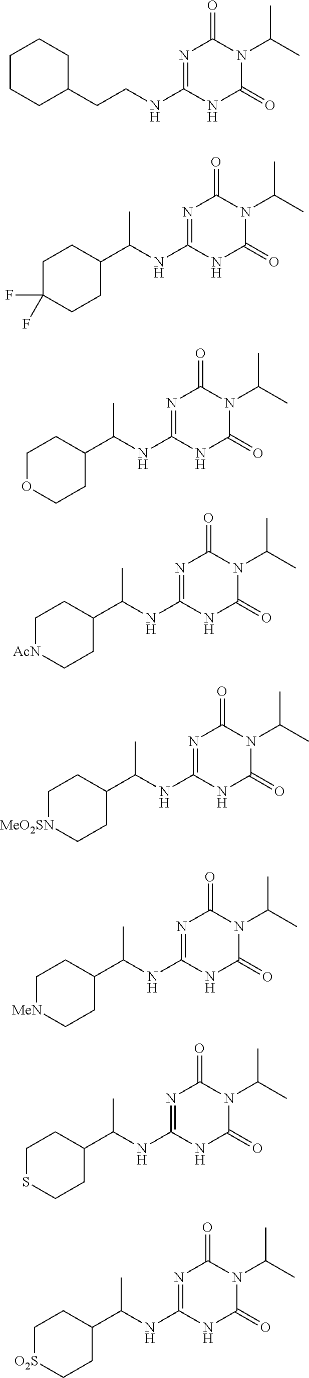 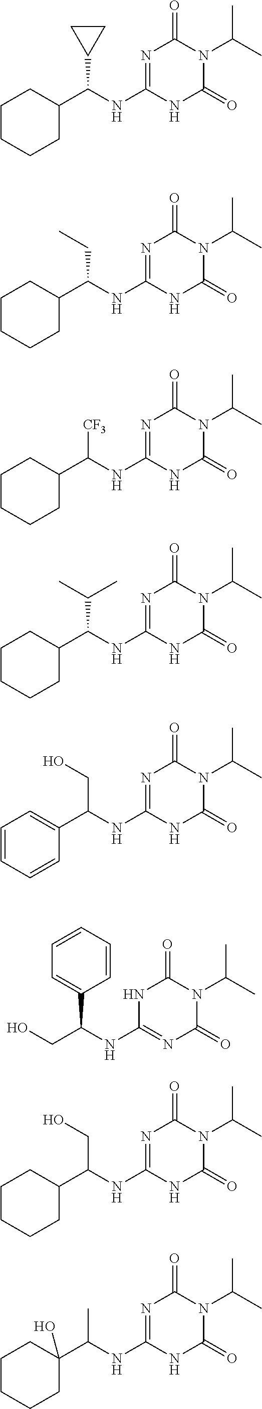 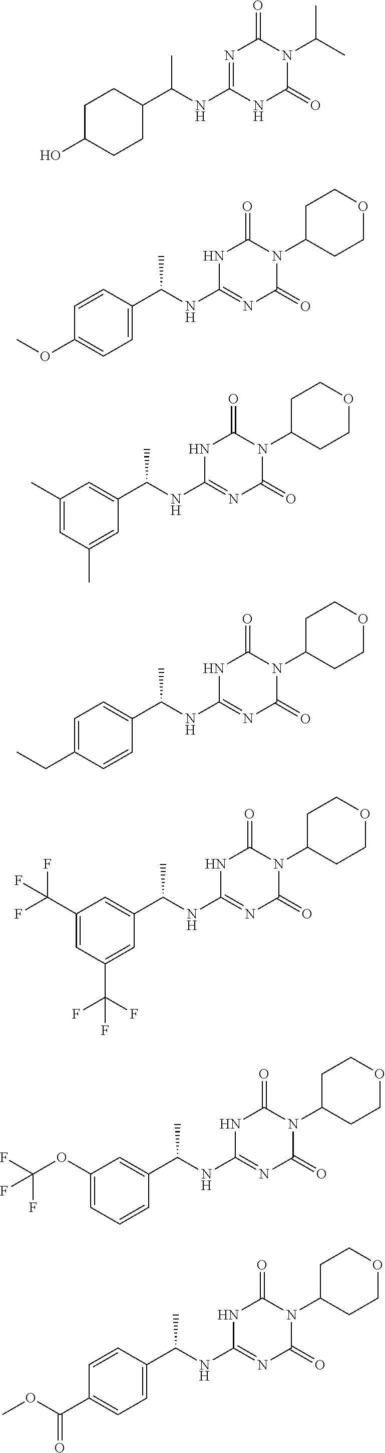 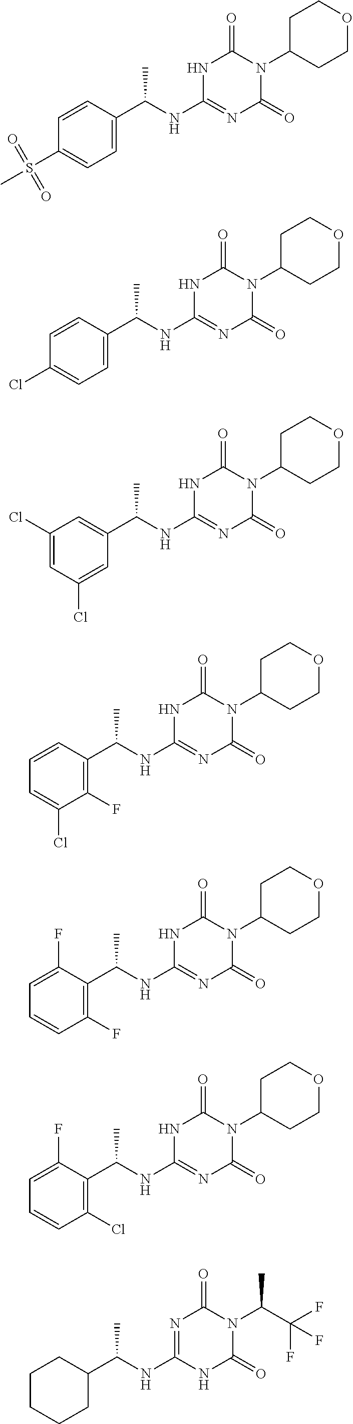 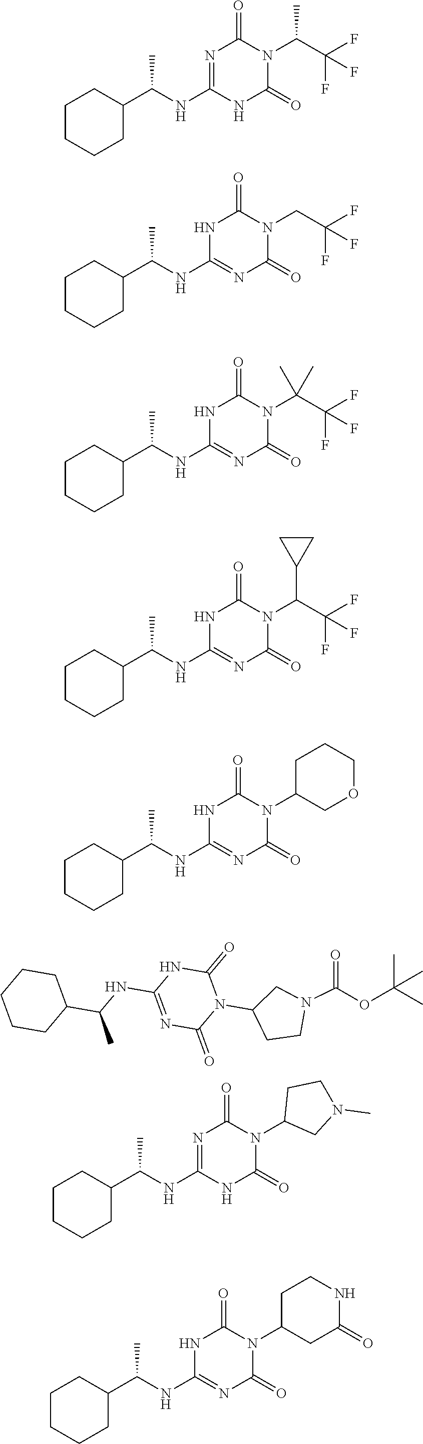 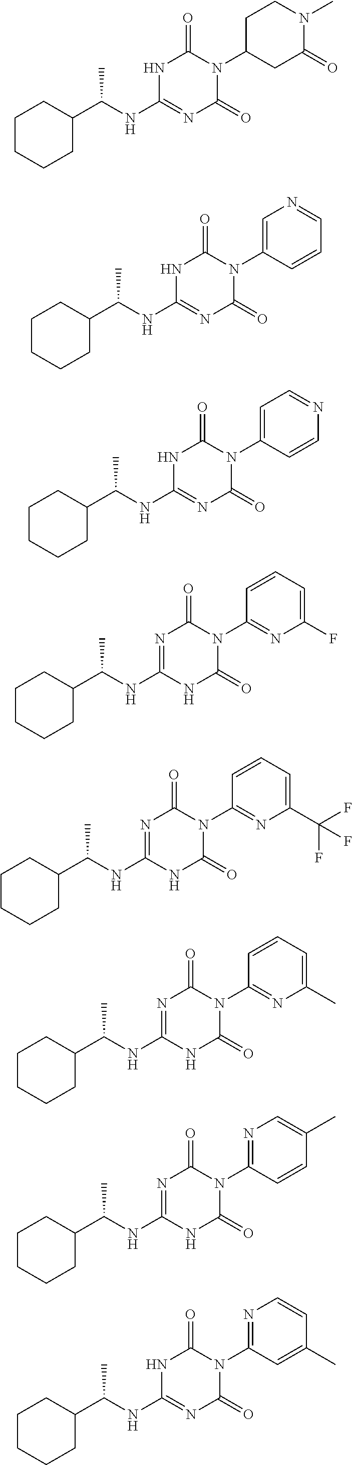 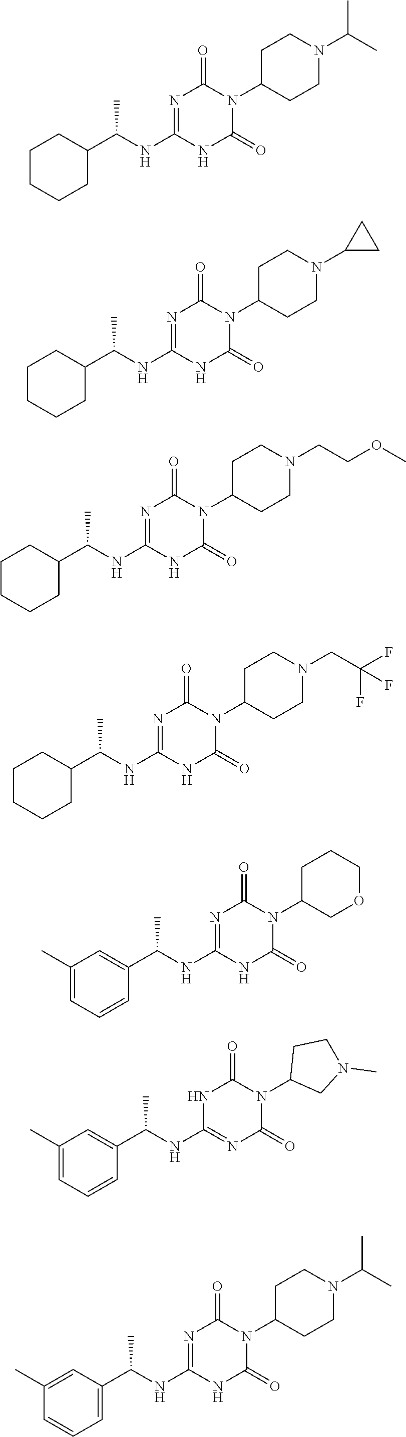 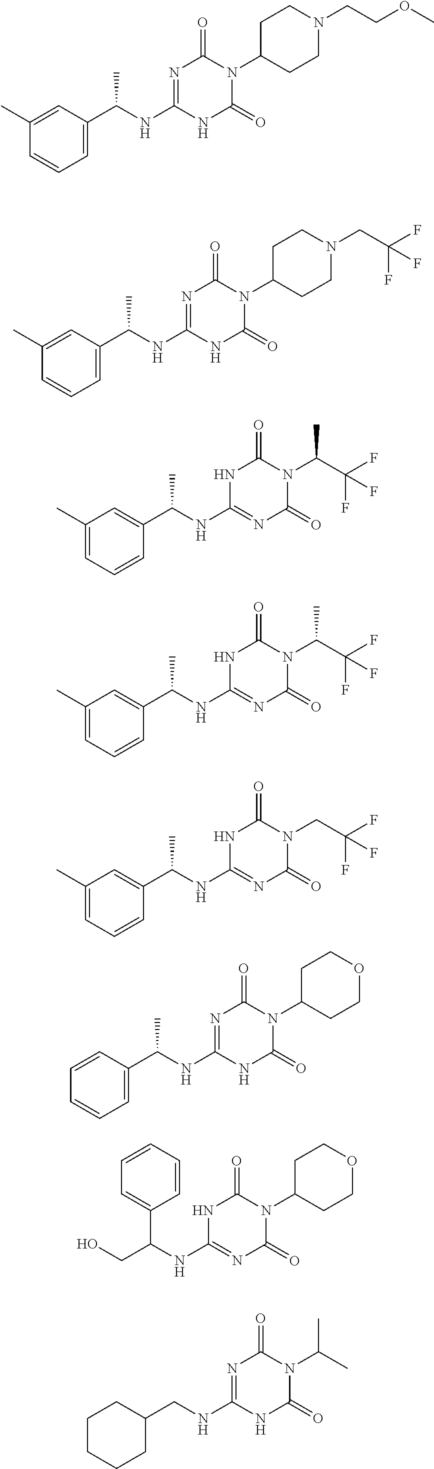 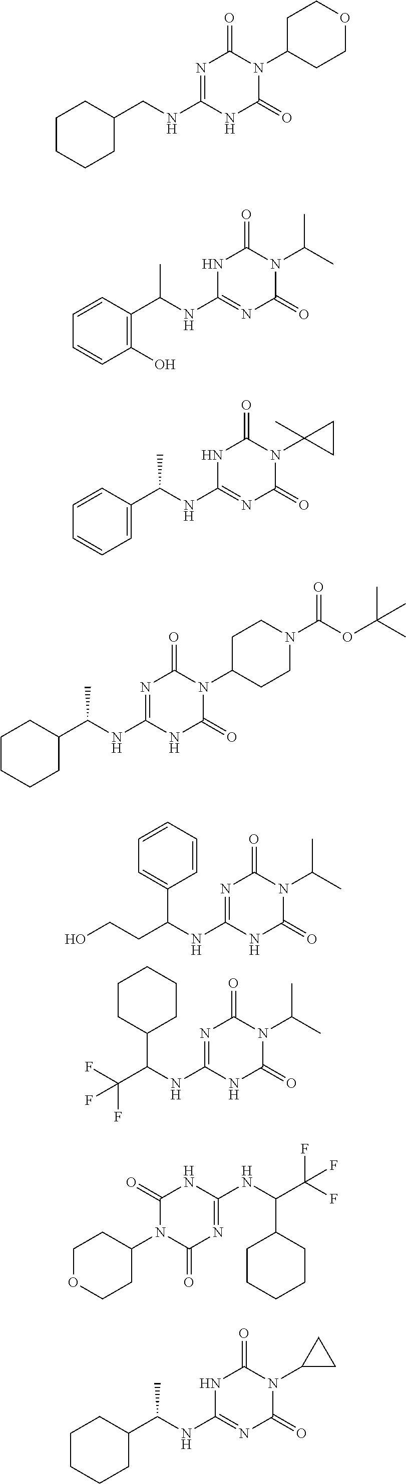 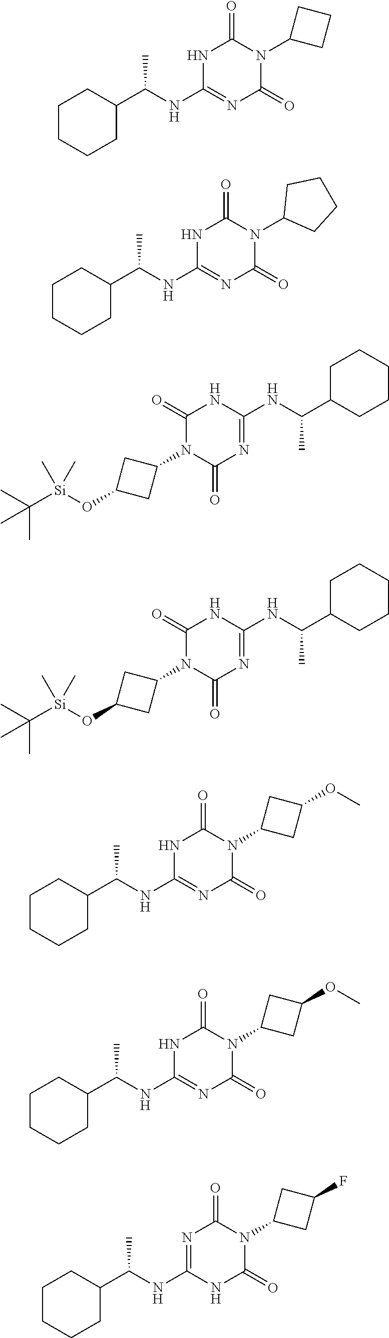 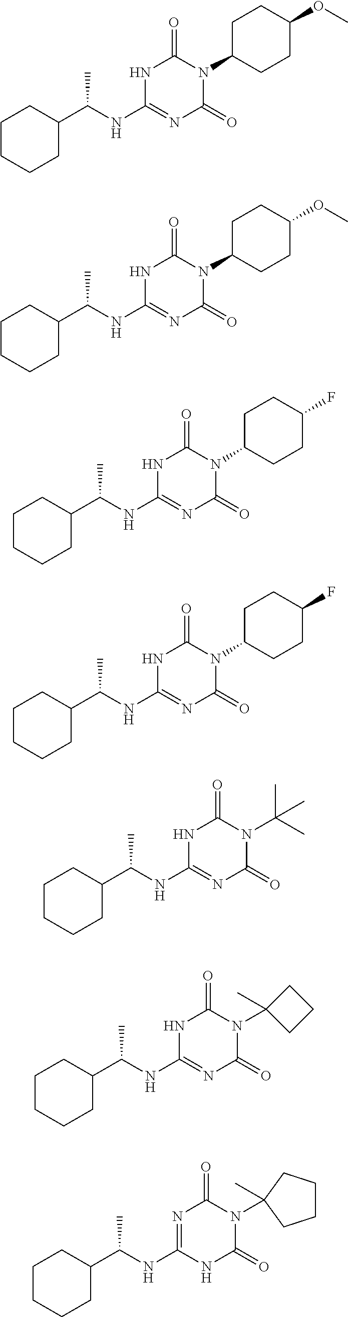 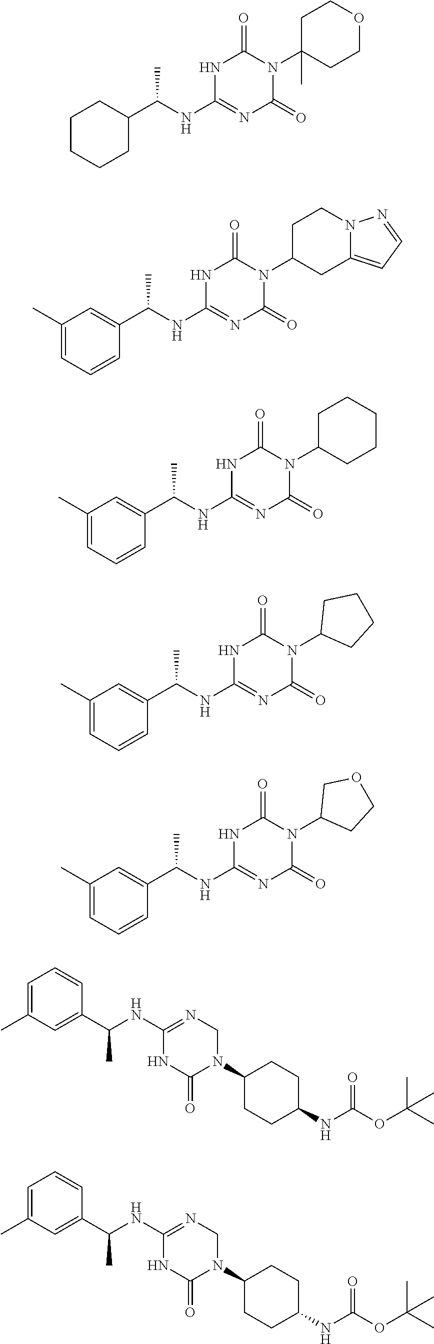 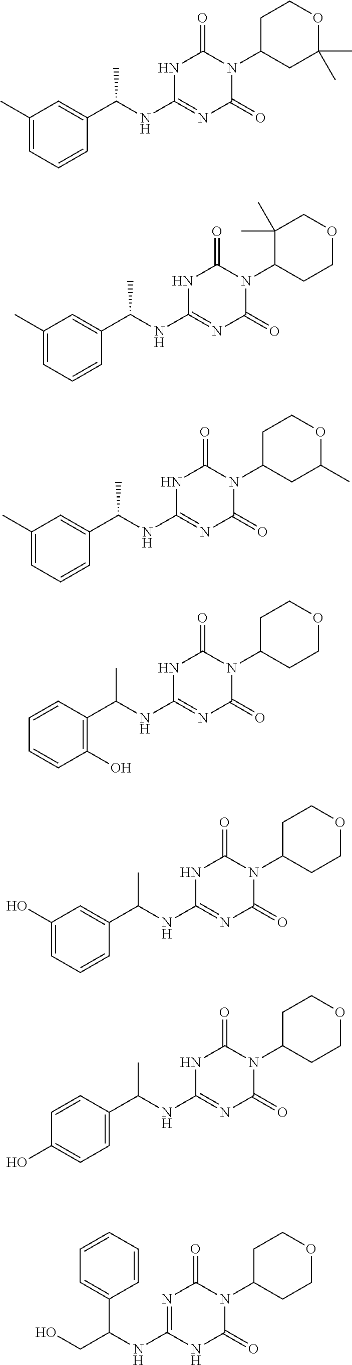 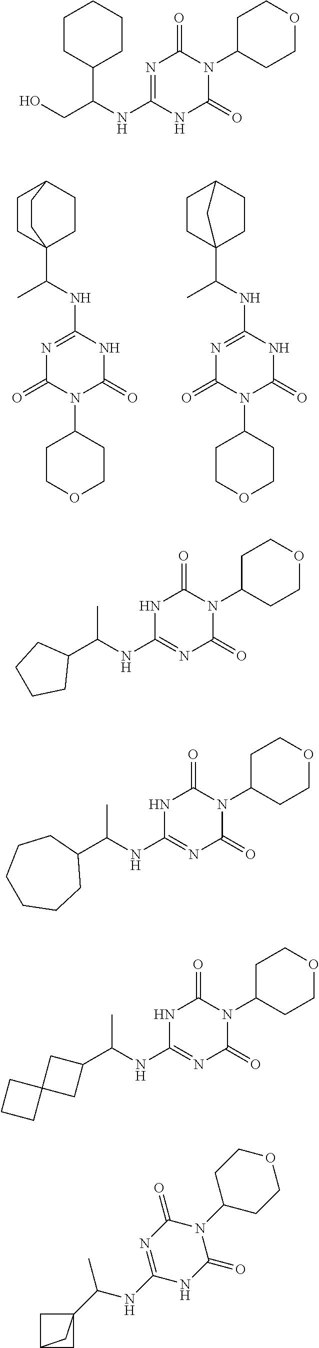 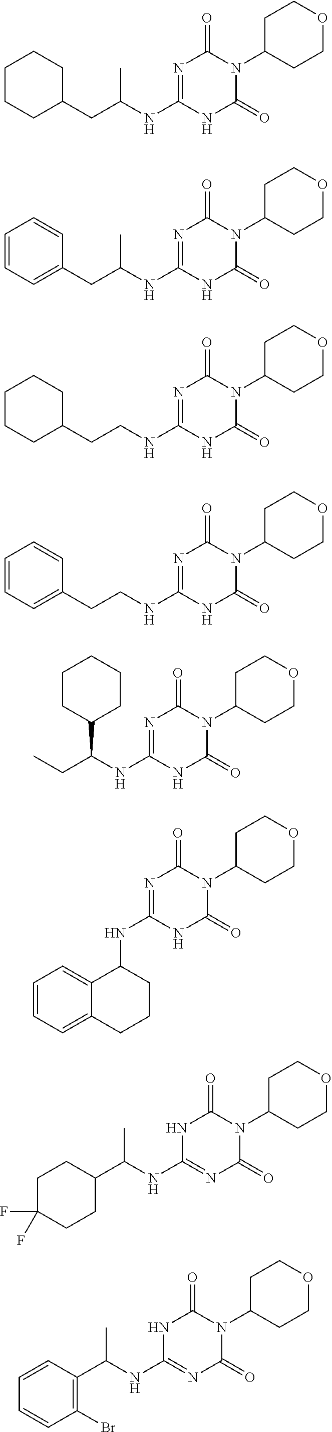 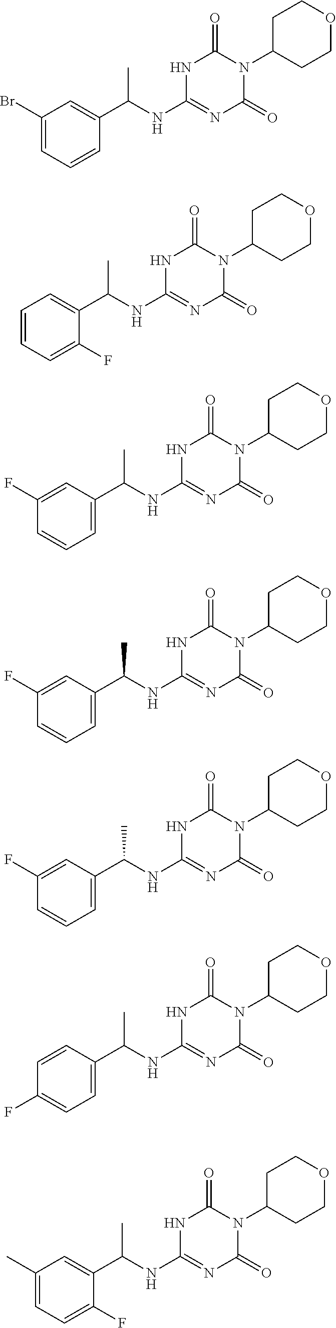 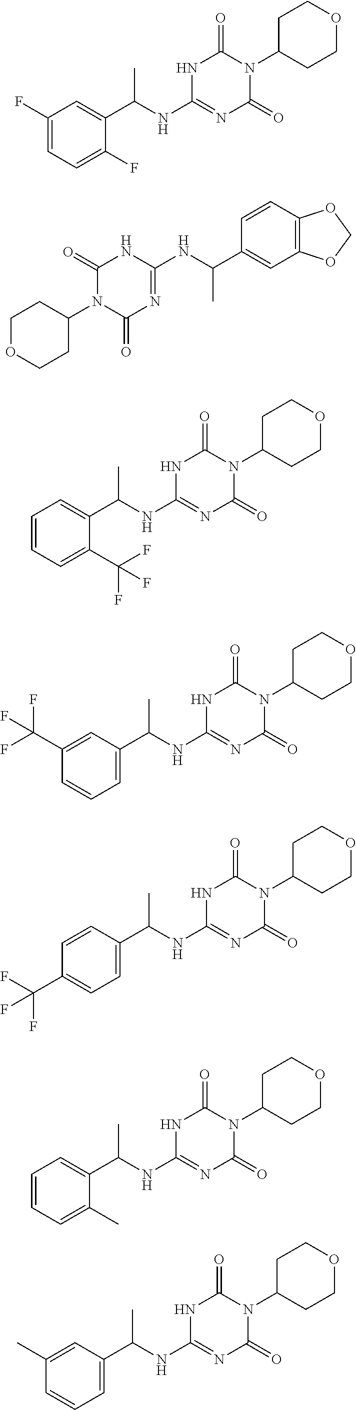 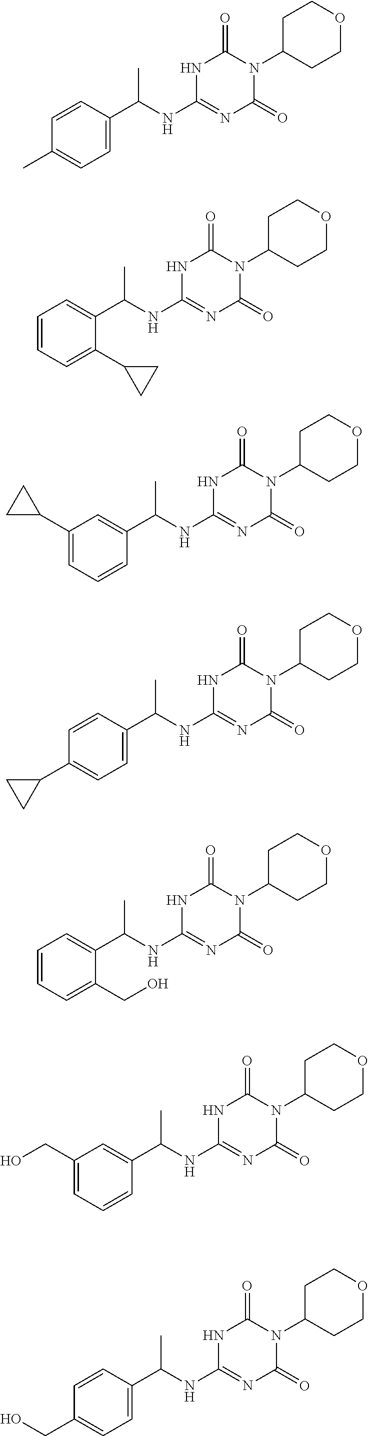 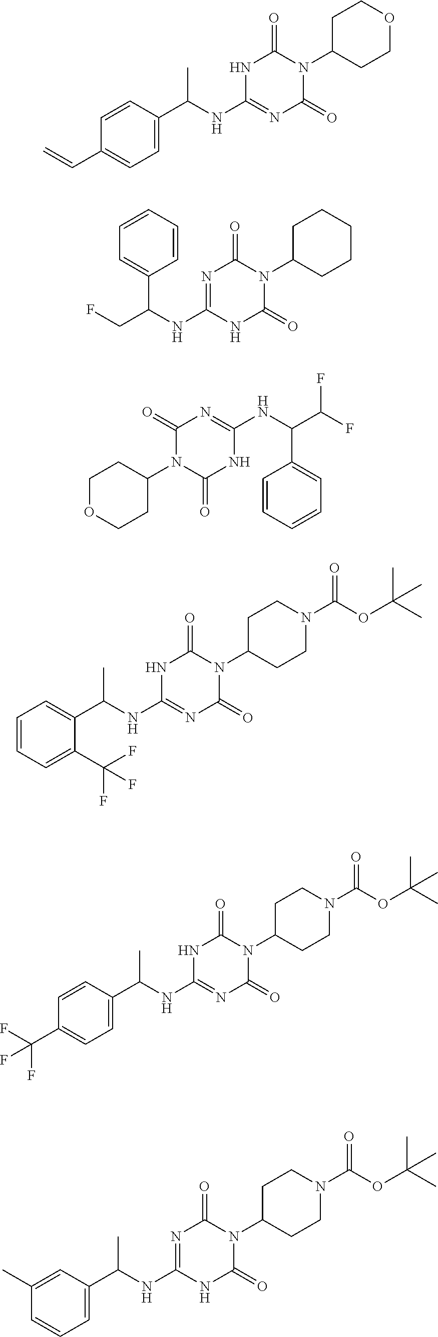 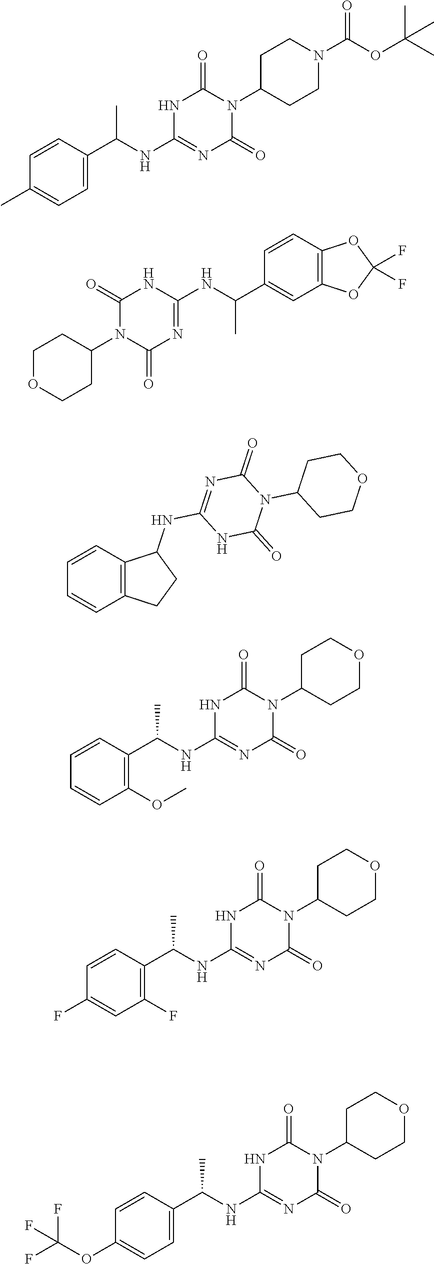 [SMILES: Cc1ccc(C(C)Nc2nc(=O)n(C3CCN(C(=O)OC(C)(C)C)CC3)c(=O)[nH]2)cc1 ; CC(Nc1nc(=O)n(C2CCOCC2)c(=O)[nH]1)c1ccc2c(c1)OC(F)(F)O2 ; O=c1nc(NC2CCc3ccccc32)[nH]c(=O)n1C1CCOCC1 ; COc1ccccc1[C@H](C)Nc1nc(=O)n(C2CCOCC2)c(=O)[nH]1 ; C[C@H](Nc1nc(=O)n(C2CCOCC2)c(=O)[nH]1)c1ccc(F)cc1F ; C[C@H](Nc1nc(=O)n(C2CCOCC2)c(=O)[nH]1)c1ccc(OC(F)(F)F)cc1] 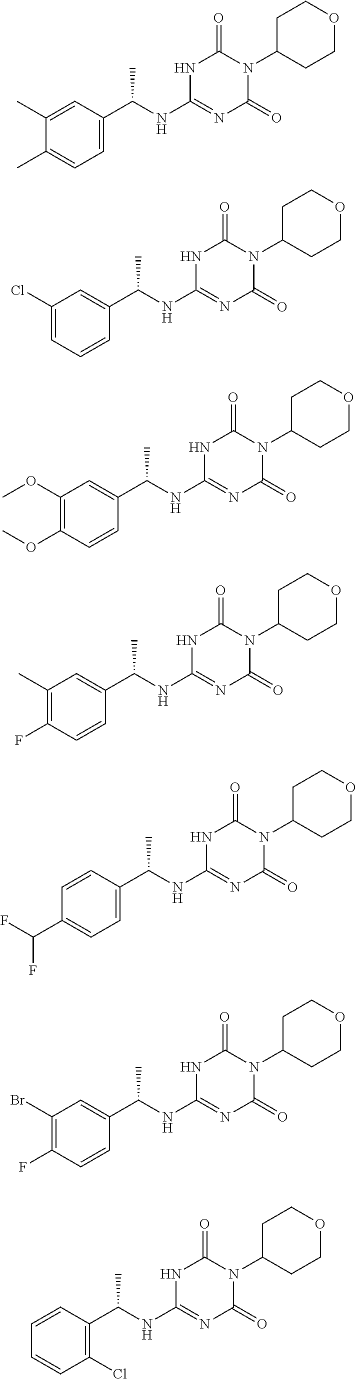 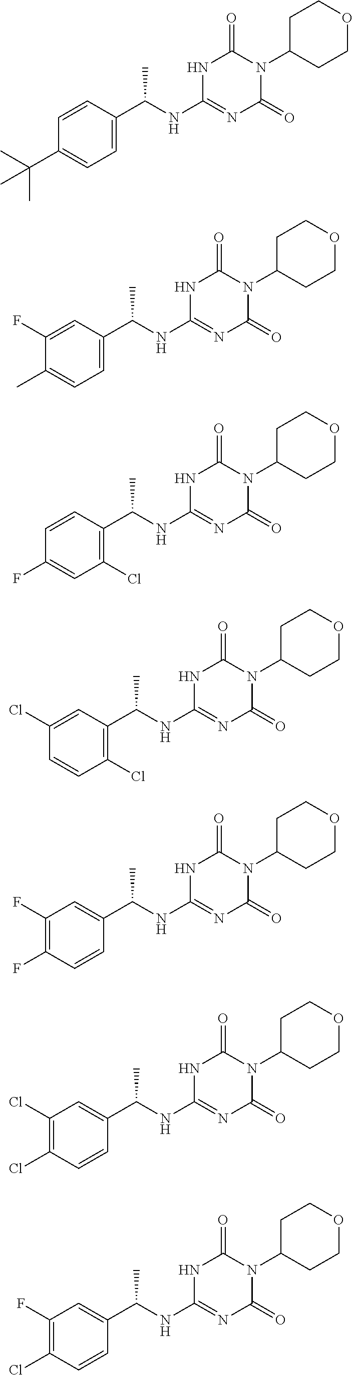 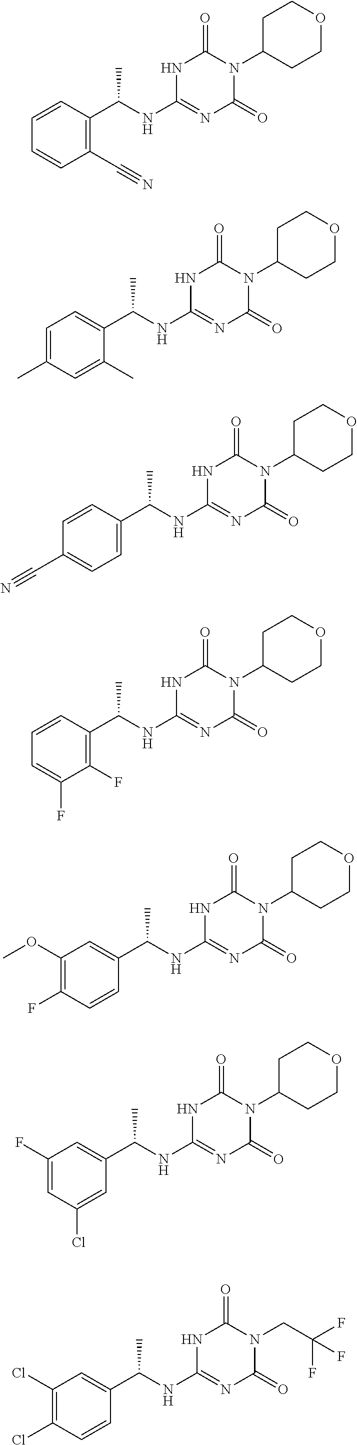 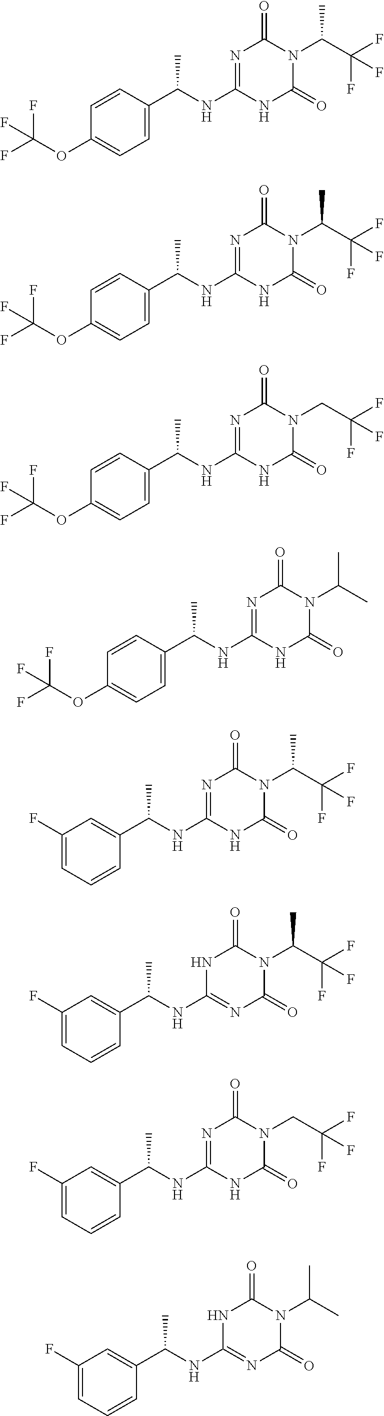 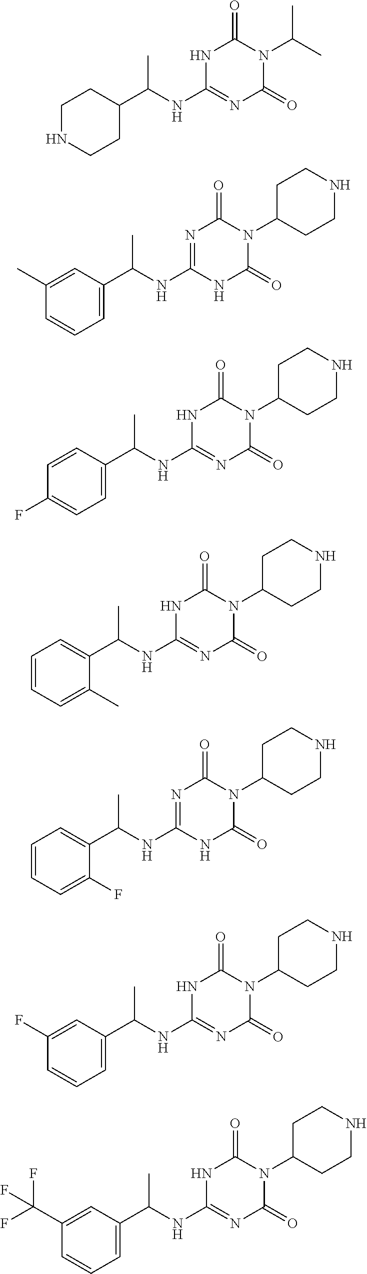 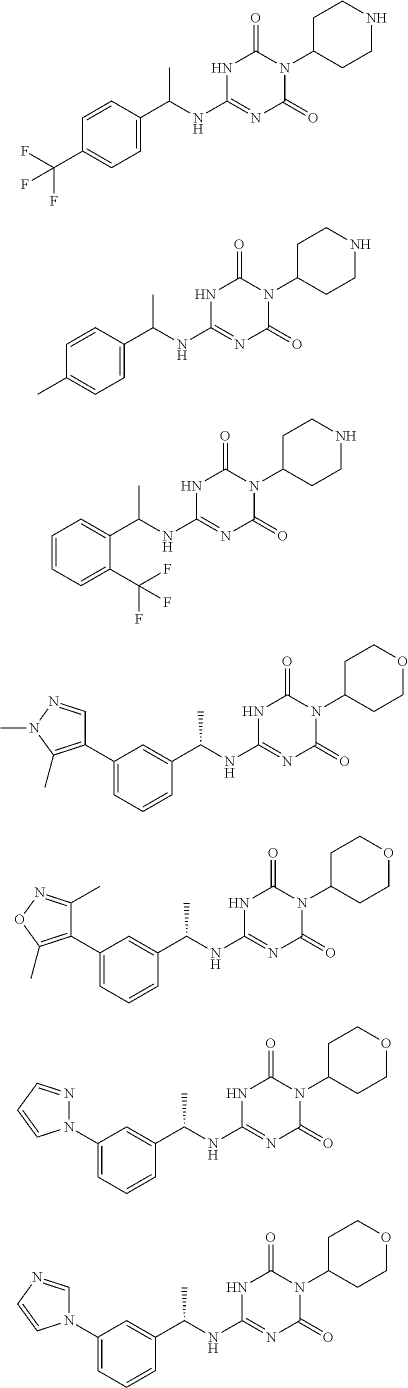 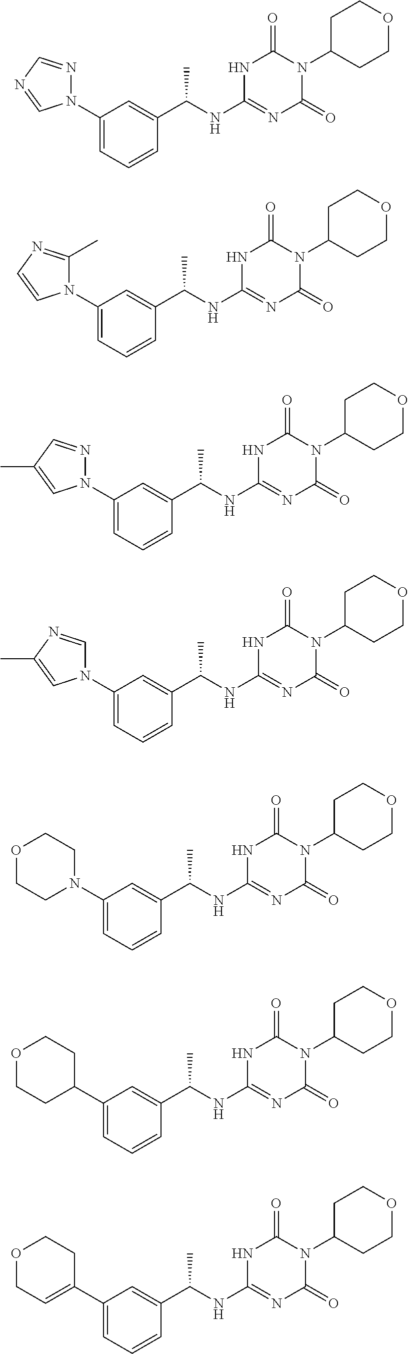 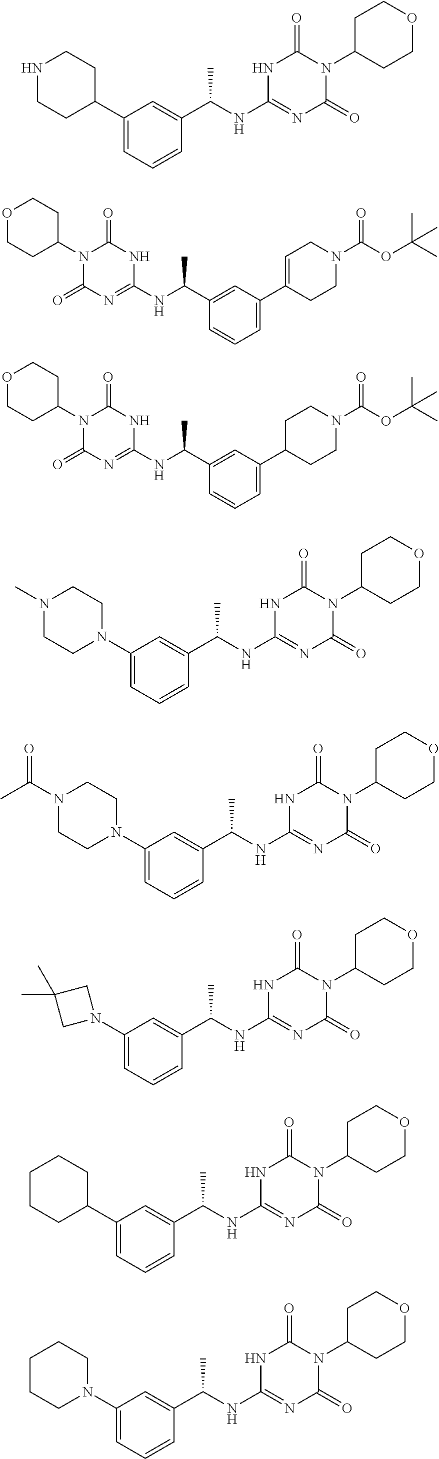 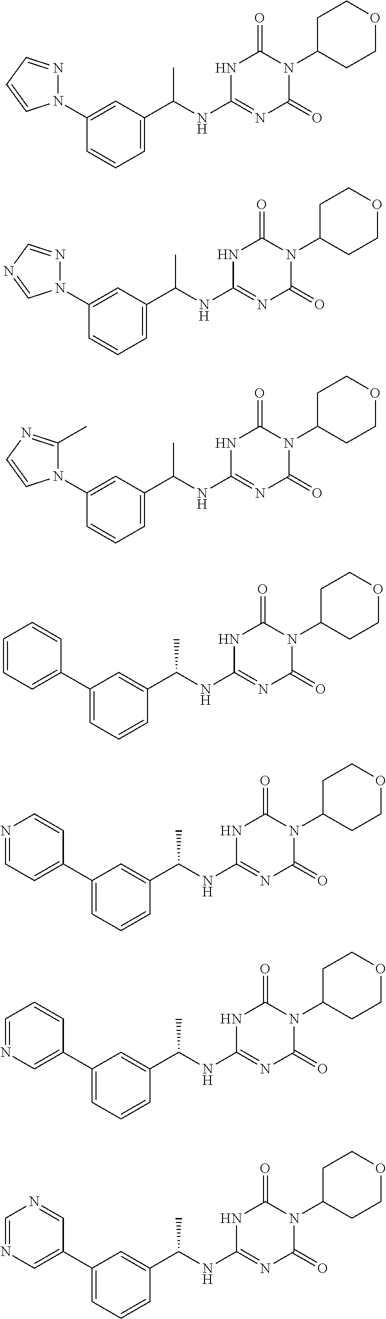 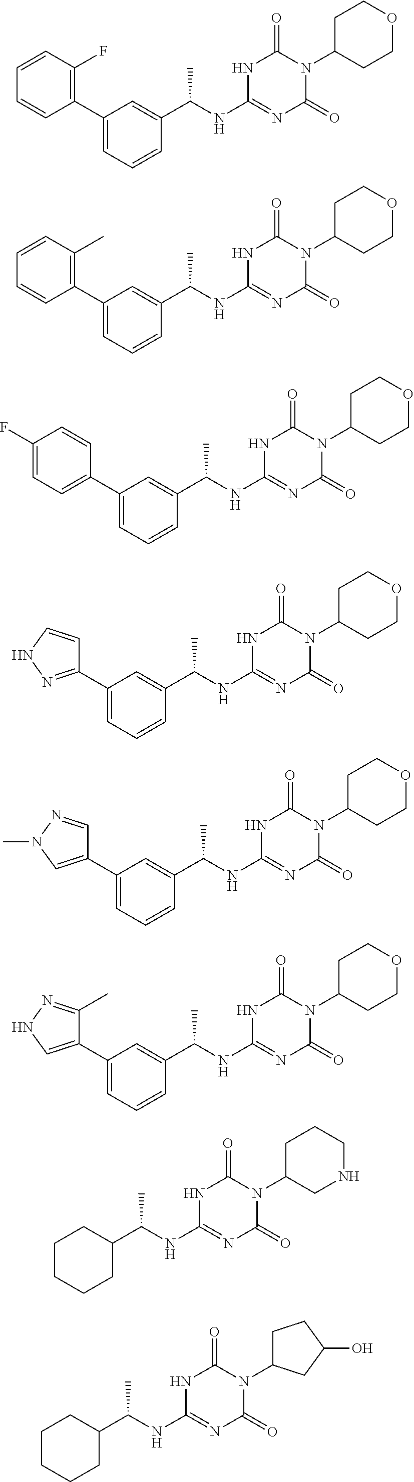 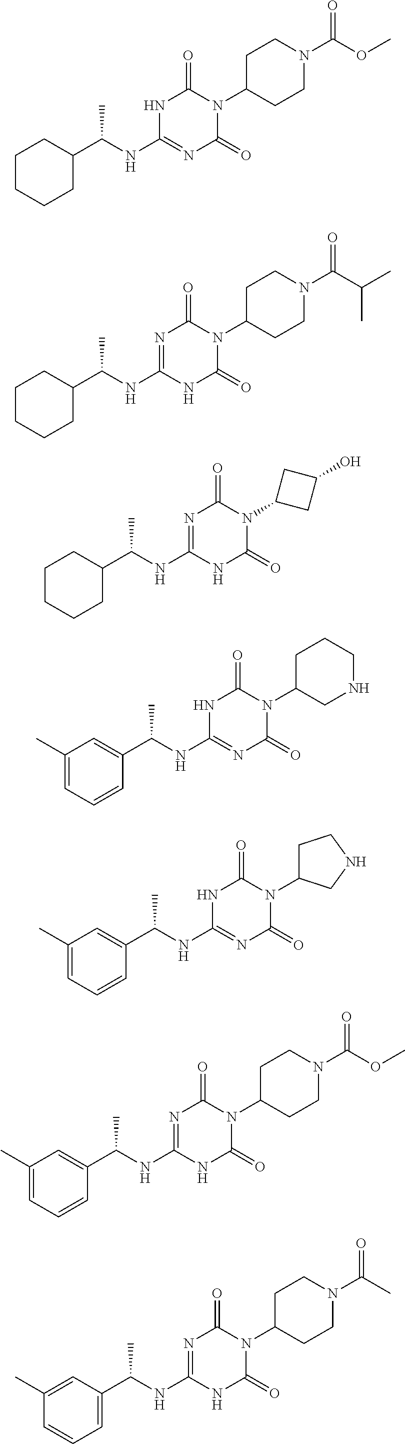 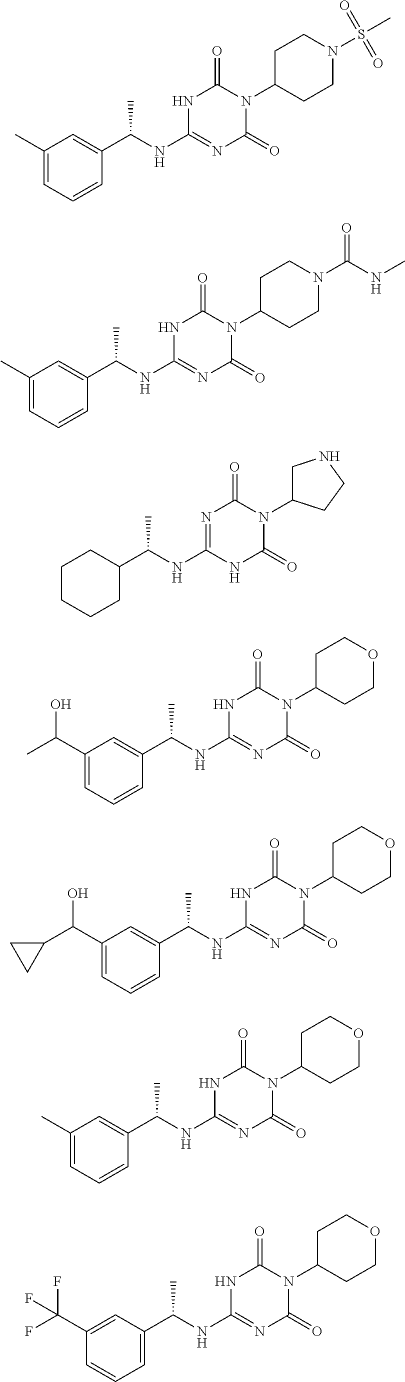 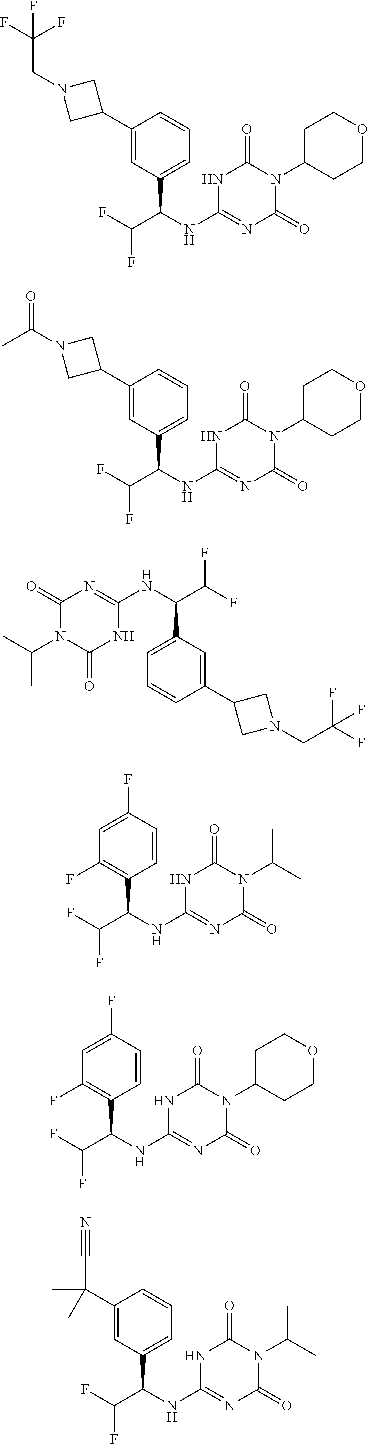 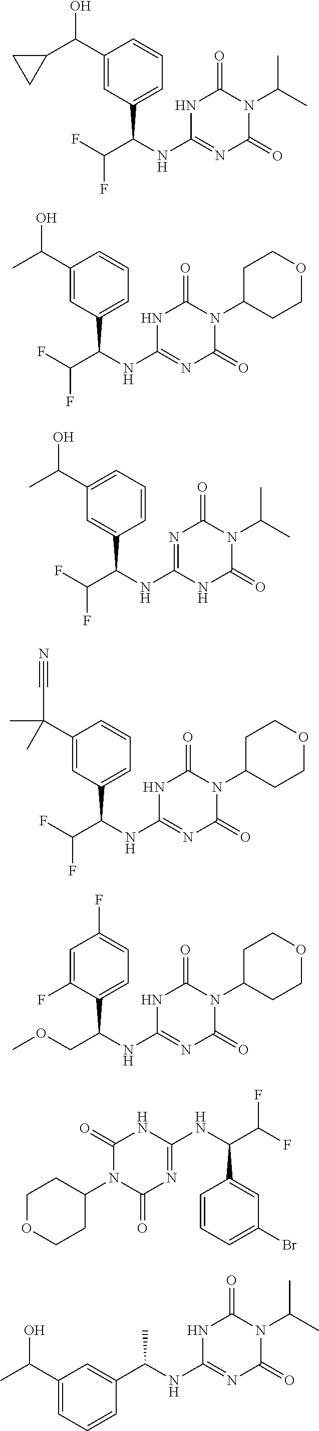 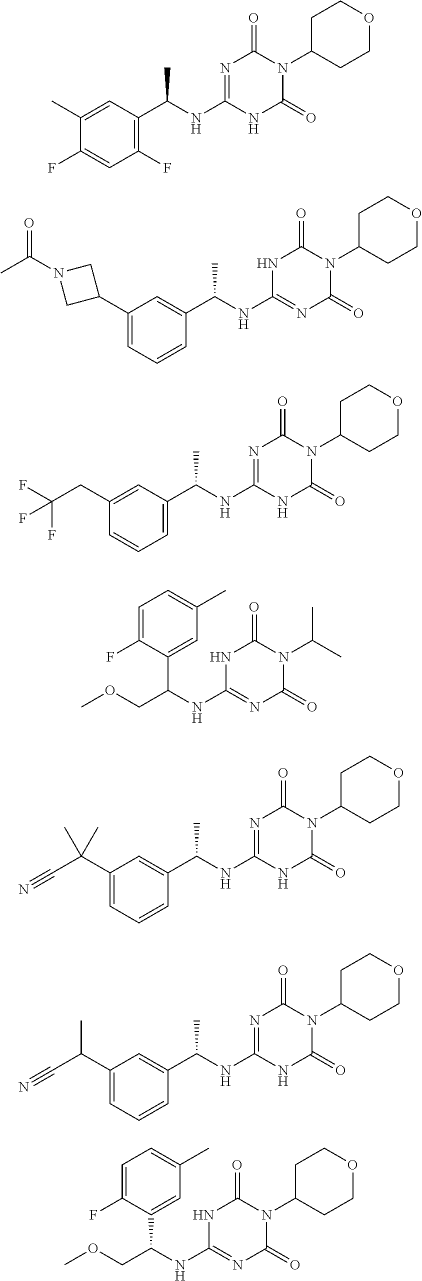 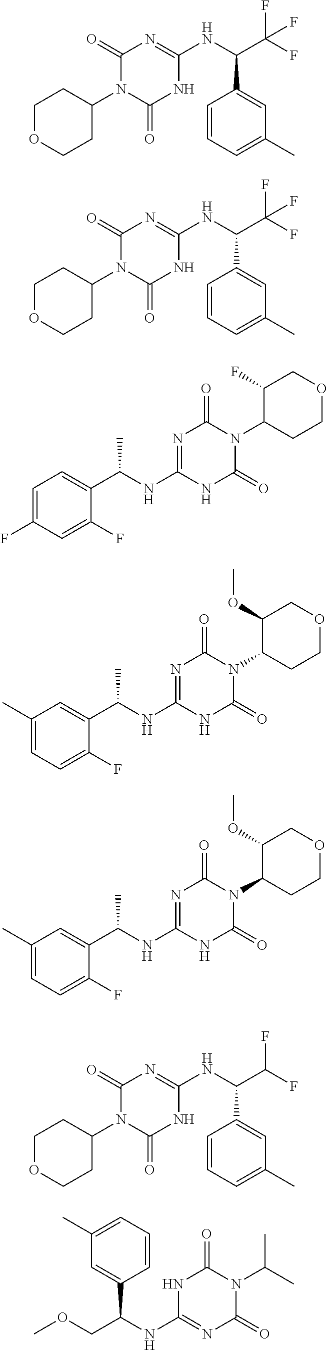 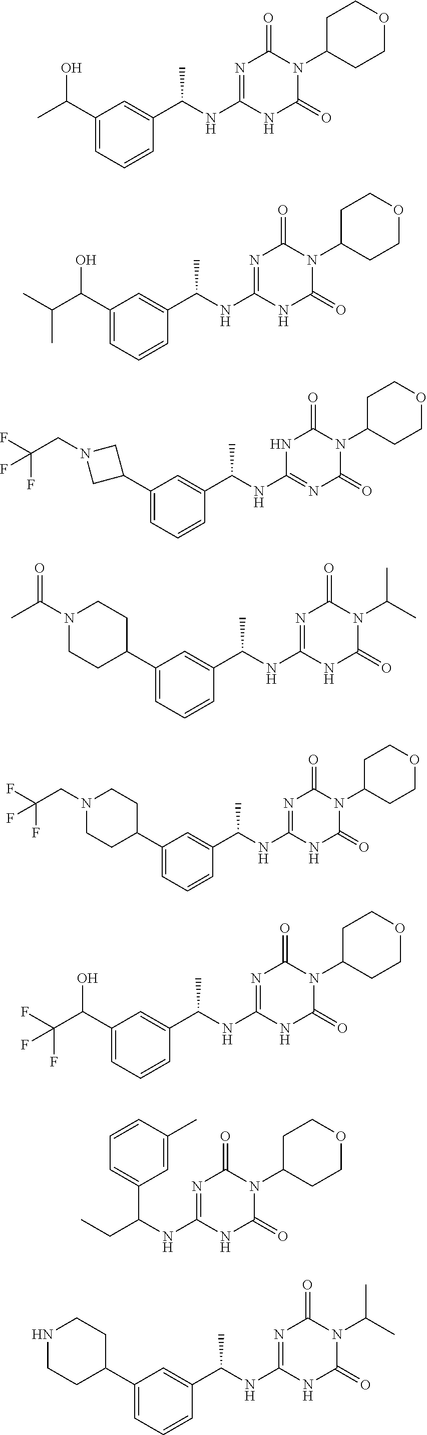 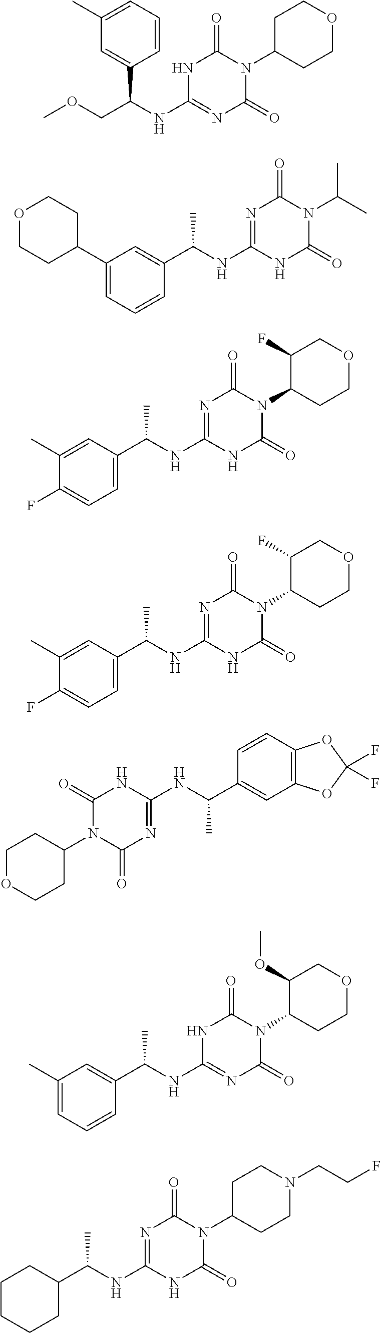 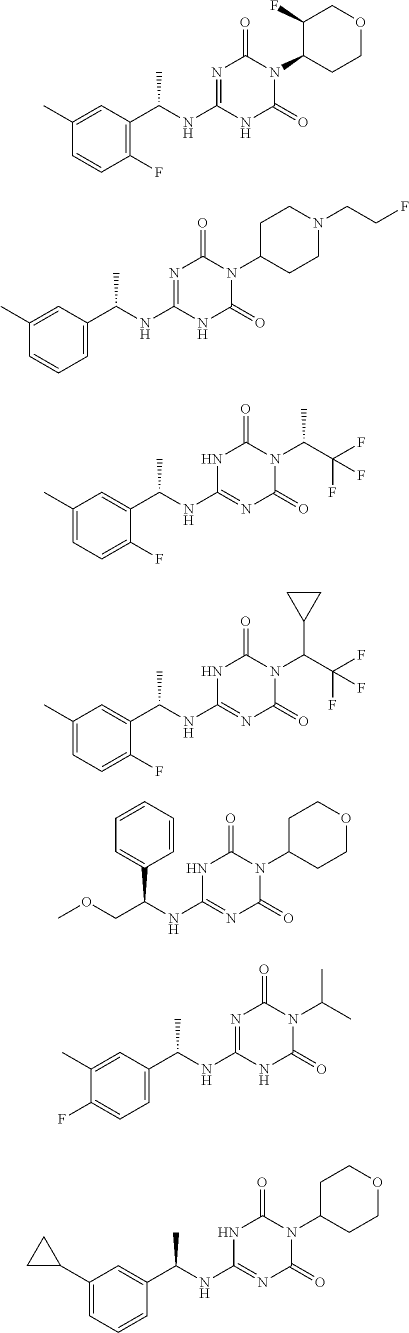 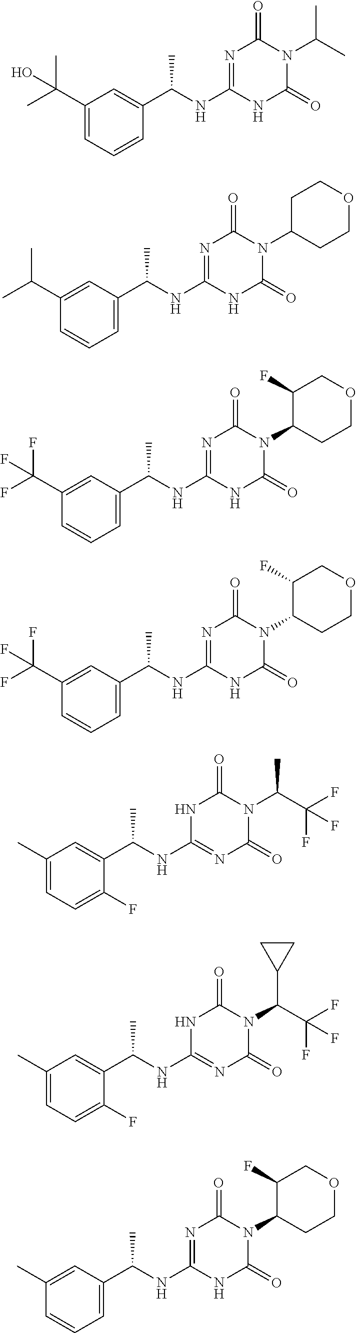 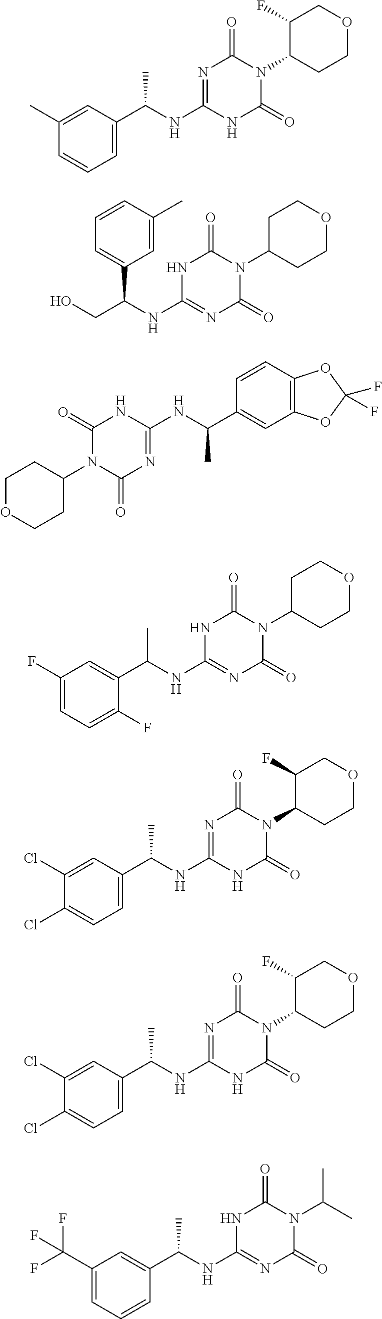 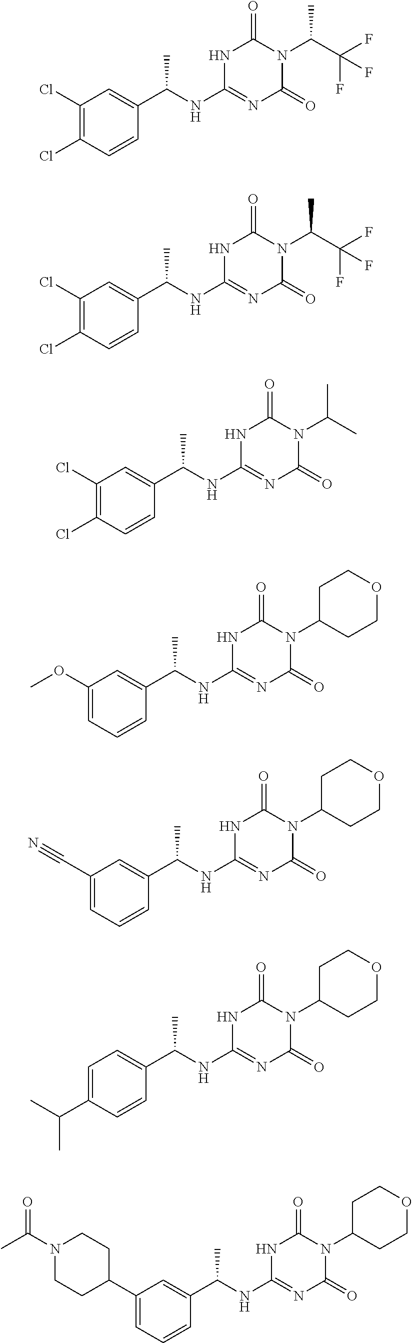 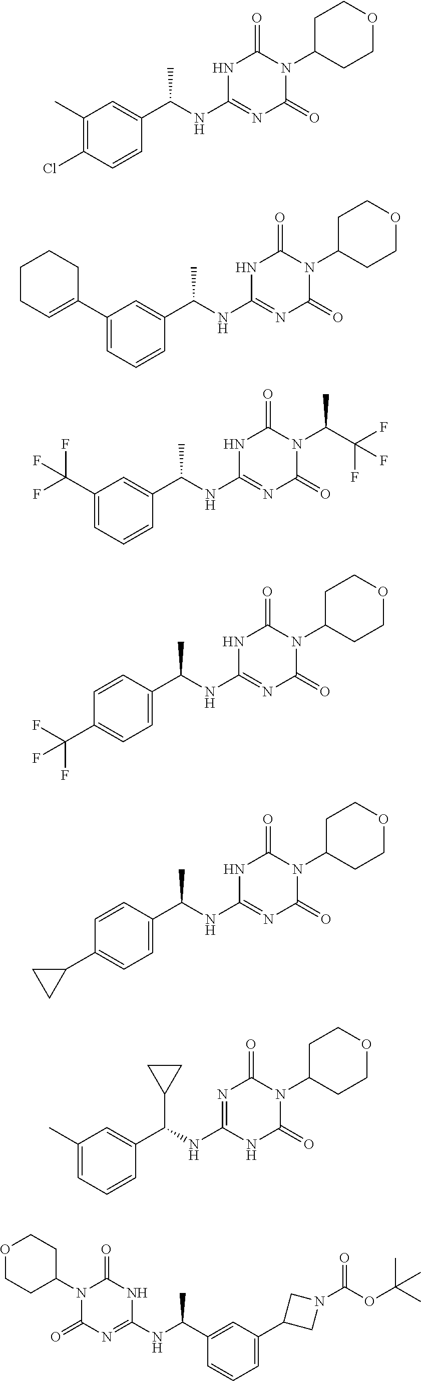 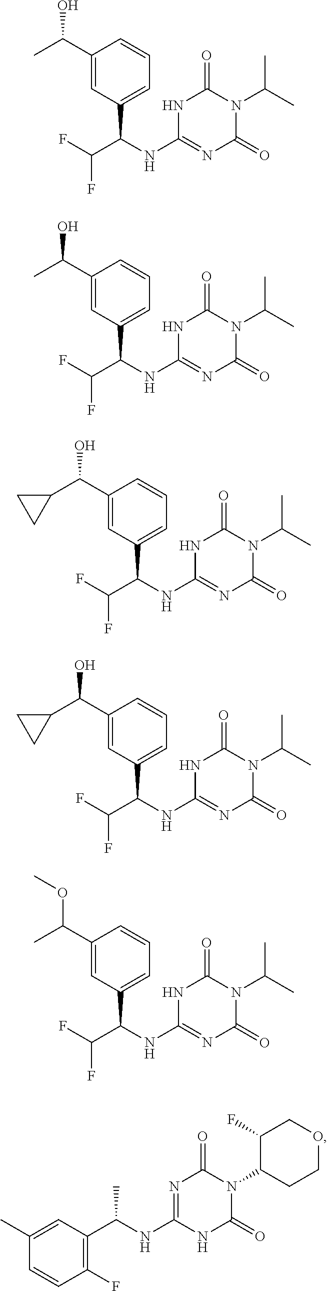
tautomer, cis- or trans-isomer, mesomer, racemate, enantiomer, diastereomer, or mixture thereof, or a pharmaceutically acceptable salt solvate, or prodrug thereof.

15. A pharmaceutical composition comprising a therapeutically effective amount of the compound of claim 1, or a tautomer, cis- or trans-isomer, mesomer, racemate, enantiomer, diastereomer, or mixture thereof, or a pharmaceutically acceptable salt solvate, or prodrug thereof, and a pharmaceutically acceptable carrier.

16. A method of treating hypertrophic cardiomyopathy (HCM) or a cardiac disorder having a pathophysiological feature of HCM in a subject in need thereof, comprising administering to the subject an effective amount of the compound of claim 1, or a tautomer, cis- or trans-isomer, mesomer, racemate, enantiomer, diastereomer, or mixture thereof, or a pharmaceutically acceptable salt solvate, or prodrug thereof.

17. The method of claim 16, wherein the HCM is obstructive or nonobstructive or is caused by sarcomeric and/or non-sarcomeric mutations.

18. A method of treating a disease or disorder in a subject in need thereof, comprising administering to the subject an effective amount of a compound of claim 1, or a tautomer, cis- or trans-isomer, mesomer, racemate, enantiomer, diastereomer, or mixture thereof, or a pharmaceutically acceptable salt solvate, or prodrug thereof, wherein the disease or disorder is selected from the group consisting of heart failure with preserved ejection fraction, ischemic heart disease, angina pectoris, and restrictive cardiomyopathy.

19. A method of treating hypertrophic cardiomyopathy (HCM) or a cardiac disorder having a pathophysiological feature of HCM in a subject in need thereof, comprising administering to the subject an effective amount of the pharmaceutical composition of claim 15.

20. A method of treating a disease or disorder in a subject in need thereof, comprising administering to the subject an effective amount of the pharmaceutical composition of claim 15, wherein the disease or disorder is selected from the group consisting of heart failure with preserved ejection fraction, ischemic heart disease, angina pectoris, and restrictive cardiomyopathy.
